(19) United States
(12) Patent Application Publication
(10) Pub. No.: US 2026/0327691 A1
(43) Pub. Date: Oct. 22, 2026

(54) IMPROVEMENTS TO NEXT-GENERATION TARGET ENRICHMENT PERFORMANCE

(71) Applicant: Roche Sequencing Solutions, Inc., Pleasanton, CA (US)

(72) Inventors: Florence Crary-Dooley, Dublin, CA (US); Brian Christopher Godwin, Livermore, CA (US); Nitya Margaret Furtado, Pleasanton, CA (US); Jingchuan Li, Dublin, CA (US); Junyan Lin, San Ramon, CA (US); Donald E. Sharon, Bristol, NH (US); Ruben Gerhard van der Merwe, Kanonberg (ZA); Beijing Wu, Pleasanton, CA (US); Liu Xi, Fremont, CA (US)

(21) Appl. No.: 19/106,865

(22) PCT Filed: Aug. 28, 2023
(86) PCT No.: PCT/EP2023/073555

§ 371 Date: Feb. 26, 2025

Related U.S. Application Data
(60) Provisional application No. 63/374,400, filed on Sep. 2, 2022.

(51) Int. Cl. C12Q 1/6848; C12Q 1/6806

(52) U.S. Cl. CPC C12Q 1/6848; C12Q 1/6806

(57) ABSTRACT

The present disclosure is directed to compositions and kits for PCR amplification. The present disclosure is also directed to methods of amplifying nucleic acid molecules to improve upon uniformity of coverage and/or to reduce GC bias during downstream sequencing operations.

### FIELD OF THE DISCLOSURE

The present disclosure is directed to compositions and methods for improving uniformity of coverage and/or reducing GC bias during sequencing.

### BACKGROUND OF THE DISCLOSURE

Next-generation sequencing is a massively parallel sequencing technology that offers high throughput, scalability, and speed. Next-generation sequencing is instrumental for investigating the molecular basis of diseases (e.g., cancer) given its sensitivity and specificity.

Next generation sequencing application data can result in poor performance which can include low on-target rate, low genome equivalent recovery (dedup/unique depth), and low uniformity of coverage. Low uniformity can be observed in target enrichment applications where either high GC or high AT content is being enriched. This is commonly described as GC bias. Uniformity of coverage is a key metric and impacts the ability to evenly enrich and sequence regions of interest. Poor uniformity of coverage results in less sequencing coverage in poor performing regions which ultimately requires more sequencing to achieve target sequencing depth in these poorer performing regions. Poor on-target rate also leads to the need for more sequencing and ultimately a higher sequencing cost per sample.

Preparing nucleic acid molecules for next-generation sequencing involves multiple library preparation steps. In general, the nucleic acid molecule of interest is obtained, purified, fragmented, end-repaired, and A-tailed; adapters are then ligated; and then the libraries are purified and/or enriched, quantitated, normalized, and loaded onto the instrument. GC bias may be introduced during PCR amplification of a nucleic library and/or during enrichment of the nucleic acid library. Therefore, there is a need in the art for a method to reduce GC bias during amplification and/or target enrichment in NGS library preparation workflows.

### BRIEF SUMMARY OF THE DISCLOSURE

It is desirable to reduce the generation of GC bias during amplification and/or library enrichment. The present disclosure is directed to compositions and methods for improving uniformity of coverage and/or reducing GC bias during sequencing, especially in reducing GC bias during sequencing of target enriched samples. In particular, Applicant has unexpectedly discovered that the use of primers having high melting temperatures and/or compositions which include one or more enhancers improves the uniformity of coverage and/or reduces GC bias during one or more downstream sequencing operations. Applicant has also unexpectedly discovered that uniformity of coverage may be improved by optimizing temperature and time/temporal parameters during capture extension. These and other improvements will be described in further detail herein.

A first aspect of the present disclosure is a composition comprising a polymerase, one or more primers, unmodified dNTPs, and at least one enhancer.

In some embodiments, the at least one enhancer is selected from betaine, dimethyl sulfoxide (DMSO), a disaccharide, and a single stranded DNA binding protein (SSB).

In some embodiments, the at least one enhancer is betaine. In some embodiments, a concentration of betaine in the composition ranges from between about 0.2 mM to about 0.8 mM. In some embodiments, a concentration of betaine in the composition ranges from between about 0.3 mM to about 0.6 mM. In some embodiments, a concentration of betaine in the composition is about 0.5 mM.

In some embodiments, the at least one enhancer is DMSO. In some embodiments, an amount of DMSO in the composition ranges from between about 1% (v/v) to about 10% (v/v). In some embodiments, an amount of DMSO in the composition ranges from between about 2% (v/v) to about 9% (v/v).

In some embodiments, the one or more primers have a melting temperature (Tm) ranging from between about 57° C. to about 95° C. In some embodiments, the one or more primers have a Tm ranging from between about 57° C. to about 85° C. In some embodiments, the one or more primers have a Tm ranging from between about 57° C. to about 75° C. In some embodiments, the one or more primers have a Tm ranging from between about 57° C. to about 72° C. In some embodiments, the one or more primers have a Tm ranging from between about 57° C. to about 69° C. In some embodiments, the one or more primers have a Tm ranging from between about 57° C. to about 66° C. In some embodiments, the one or more primers have a Tm ranging from between about 57° C. to about 63° C. In some embodiments, up to about 10% of the primers in the composition are high melting temperature and/or high GC content primers (e.g., pre-amplification primers, capture primers, release primers, amplification primers, etc.).

In some embodiments, the one or more primers comprise one or more modified dNTPs. In some embodiments, the one or more modified dNTPs are selected from modified dGTPs and modified dATPs. In some embodiments, further comprising one or more modified dNTPs. In some embodiments, a concentration of the one or more modified dNTPs in the composition is about the same as a concentration of the unmodified dNTPs in the composition. In some embodiments, a concentration of the one or more modified dNTPs in the composition ranges from about 0.1 mM to about 0.5 mM. In some embodiments, a concentration of the one or modified dNTPs in the composition ranges from about 0.2 mM to about 0.4 mM. In some embodiments, the one or more modified dNTPs are modified dGTPs. In some embodiments, the modified dGTPs comprise 7-Deaza-2′-deoxyguanosine-5′-Triphosphates. In some embodiments, the one or more modified dNTPs are modified dATPs. In some embodiments, the modified dATPs comprise 2-Amino-2′ deoxyadenosine-5′-Triphosphates.

In some embodiments, the composition further comprises a divalent cation. In some embodiments, the divalent cation is selected from the group consisting of Co²⁺, Mn²⁺, Mg²⁺, Cd²⁺, and Ca²⁺.

In some embodiments, the composition further comprises one or more buffers. In some embodiments, the composition further comprises one or more polyols.

In some embodiments, the one or more primers comprise pre-capture forward and reverse primers. In some embodiments, the one or more primers comprise capture primers. In some embodiments, the capture primers comprise a capture moiety.

In some embodiments, the composition further comprises one or more nucleic acid molecules. In some embodiments, the one or more nucleic acid molecules comprise a library of nucleic acid molecules, where each nucleic acid molecule in the library of nucleic acid molecules comprises first and second adapters. In some embodiments, the one or more primers comprise one or more capture primers, and wherein the one or more capture primers are capable of hybridizing to target nucleic acid sequences within the library of nucleic acid molecules.

A second aspect of the present disclosure is a composition comprising a polymerase, one or more primers, dNTPs, and optionally at least one enhancer, wherein the one or more primers include no limitation on the percent of guanine or cytosine bases, and wherein at least one primer of the one or more primers has a melting temperature greater than 63° C. In some embodiments, the at least one primer has a melting temperature greater than about 69° C. In some embodiments, the at least one primer has a melting temperature greater than about 75° C. In some embodiments, the at least one primer has a melting temperature greater than about 85° C. In some embodiments, the at least one primer has a melting temperature greater than about 95° C. In some embodiments, up to about 10% of the primers in the composition are high melting temperature and/or high GC content primers (e.g., pre-amplification primers, capture primers, release primers, amplification primers, etc.).

In some embodiments, the at least one optional enhancer is selected from betaine, DMSO, a disaccharide, and a single stranded DNA binding protein (SSB). In some embodiments, the at least one optional enhancer is betaine, and wherein a concentration of the betaine in the composition ranges from between about 0.2 mM to about 0.8 mM.

In some embodiments, the dNTPs comprise a mixture of unmodified dNTPs and modified dNTPs. In some embodiments, the one or more modified dNTPs comprise 7-Deaza-2′-deoxyguanosine-5′-Triphosphate. In some embodiments, the one or more modified dNTPs comprise 2-Amino-2′ deoxyadenosine-5′-Triphosphate. In some embodiments, a concentration of the one or more modified dNTPs in the composition ranges from between about 0.2 mM to about 0.8 mM. In some embodiments, the one or more primers comprise one or more modified dNTPs. In some embodiments, the one or more modified dNTPs are selected from the group consisting of 7-Deaza-2′-deoxyguanosine-5′-Triphosphate and 2-Amino-2′ deoxyadenosine-5′-Triphosphate.

In some embodiments, the composition further comprises a divalent cation. In some embodiments, the divalent cation is selected from the group consisting of Co2+, Mn2+, Mg2+, Cd2+, and Ca2+. In some embodiments, the composition further comprises one or more buffers. In some embodiments, the composition further comprises one or more polyols.

In some embodiments, the one or more primers comprise pre-capture forward and reverse primers. In some embodiments, the one or more primers are capture primers. In some embodiments, the composition further comprises one or more nucleic acid molecules.

In some embodiments, the one or more nucleic acid molecules comprise first and second adapters. In some embodiments, the one or more primers comprise one or more capture primers, and wherein the one or more capture primers are capable of hybridizing to target nucleic acid sequences of the nucleic acid molecules. In some embodiments, the one or more nucleic acid molecules comprise DNA.

A third aspect of the present disclosure is reaction tube (or container, vial, reaction chamber, etc.) comprising the composition of any one of the first and second aspects of the present disclosure described above or as disclosed herein.

A fourth aspect of the present disclosure is a use of a composition, such as any of the compositions noted in the first and second aspects of the disclosure described above or as further described herein, in the amplification of one or more nucleic acid molecules.

A fifth aspect of the present disclosure is a composition comprising one or more primers, input nucleic acid molecules, and at least one enhancer selected from the group consisting of betaine, DMSO, a disaccharide, and a single stranded DNA binding protein (SSB).

In some embodiments, the at least one enhancer is betaine. In some embodiments, a concentration of betaine in the composition ranges from between about 0.2 mM to about 0.8 mM. In some embodiments, a concentration of betaine in the composition ranges from between about 0.3 mM to about 0.6 mM. In some embodiments, the at least one enhancer is DMSO.

In some embodiments, an amount of DMSO in the composition ranges from between about 1% (v/v) to about 10% (v/v). In some embodiments, an amount of DMSO in the composition ranges from between about 2% (v/v) to about 8% (v/v).

In some embodiments, the one or more primers have a Tm ranging from between about 57° C. to about 95° C. In some embodiments, the one or more primers have a Tm ranging from between about 57° C. to about 85° C. In some embodiments, the one or more primers have a Tm ranging from between about 57° C. to about 75° C. In some embodiments, the one or more primers have a Tm ranging from between about 57° C. to about 72° C. In some embodiments, the one or more primers have a Tm ranging from between about 57° C. to about 69° C. In some embodiments, the one or more primers have a Tm ranging from between about 57° C. to about 66° C. In some embodiments, up to about 10% of the primers in the composition are high melting temperature and/or high GC content primers (e.g., pre-amplification primers, capture primers, release primers, amplification primers, etc.).

In some embodiments, the one or more primers comprise one or more modified dNTPs. In some embodiments, the one or more modified dNTPs are selected from the group consisting of modified dGTPs and modified dATPs.

In some embodiments, the composition further comprises a divalent cation. In some embodiments, the divalent cation is selected from the group consisting of Co²⁺, Mn²⁺, Mg²⁺, Cd²⁺, and Ca²⁺. In some embodiments, the composition further comprises one or more buffers.

In some embodiments, the input nucleic acid molecules comprise a prepared nucleic acid library. In some embodiments, the input nucleic acid molecules comprise captured nucleic acid molecules. In some embodiments, the captured nucleic acid molecules comprise a complex of a nucleic acid molecule comprising a target nucleic acid sequence and an extended capture primer hybridized to at least a portion of the target nucleic acid sequence.

A sixth aspect of the present disclosure is a composition comprising input nucleic acid molecules, one or more primers, and optionally at least one enhancer, wherein the one or more primers include no limitation on the percent of guanine or cytosine bases, and wherein at least one primer of the one or more primers has a melting temperature greater than 63° C. In some embodiments, the at least one primer has a melting temperature greater than about 69° C. In some embodiments, the at least one primer has a melting temperature greater than about 75° C. In some embodiments, the at least one primer has a melting temperature greater than about 85° C. In some embodiments, the at least one primer has a melting temperature greater than about 95° C. In some embodiments, up to about 10% of the primers in the composition are high melting temperature and/or high GC content primers (e.g., pre-amplification primers, capture primers, release primers, amplification primers, etc.).

In some embodiments, rein the one or more primers comprise one or more modified dNTPs. In some embodiments, the one or more modified dNTPs are selected from 7-Deaza-2′-deoxyguanosine-5′-Triphosphate and 2-Amino-2′ deoxyadenosine-5′-Triphosphate. In some embodiments, the at least one optional enhancer is selected from betaine, DMSO, a disaccharide, and a single stranded DNA binding protein (SSB).

In some embodiments, the at least one optional enhancer is betaine, and wherein a concentration of the betaine in the composition ranges from between about 0.2 mM to about 0.8 mM. In some embodiments, the composition further comprises a divalent cation. In some embodiments, the divalent cation is selected from the group consisting of Co2+, Mn2+, Mg2+, Cd2+, and Ca2+. In some embodiments, the composition further comprises one or more buffers.

In some embodiments, the input nucleic acid molecules comprise a prepared nucleic acid library. In some embodiments, the input nucleic acid molecules comprise captured DNA. In some embodiments, the captured nucleic acid molecules comprise a complex of a nucleic acid molecule comprising a target nucleic acid sequence and an extended capture primer hybridized to at least a portion of the target nucleic acid sequence.

A seventh aspect of the present disclosure reaction tube (or container, vial, reaction chamber, etc.) comprising a composition of any of the fifth or sixth aspects of the disclosure as noted above or as further described herein.

An eighth aspect of the present disclosure is a use of the composition of any one of the fifth or sixth aspects of the present disclosure noted above or as described herein for the preferential enrichment of one or more target nucleic acid molecules in a library of nucleic acid molecules.

A ninth aspect of the present disclosure is a kit comprising (i) a set of capture primers; (ii) a set of release primers; (iii) a polymerase; (iv) dNTPs; and (v) betaine or a derivative or analog thereof. In some embodiments, the dNTPs comprise a mixture of unmodified and modified dNTPs. In some embodiments, the modified dNTPs include modified dGTPs and modified dATPs. In some embodiments, the sets of capture and release primers include one or more modified dNTPs. In some embodiments, the kit further comprises instructions to prepare a capture extension master mix, wherein the prepared capture extension master mix includes a concentration of betaine of about 0.5 mM. In some embodiments, the kit further comprises one or more buffers. In some embodiments, the kit further comprises one or more divalent cations. In some embodiments, the kit further comprises one or more polyols. In some embodiments, at least one of the sets of capture and release primers have no limits on melting temperatures and/or GC content. In some embodiments, both sets of capture and release primers have no limits on melting temperatures and/or GC content.

A tenth aspect of the present disclosure is a kit comprising (i) a set of capture primers; (ii) a set of release primers; (iii) a polymerase; and (iv) dNTPs; wherein at least one primer of the set of capture primers has a melting temperature greater than about 65° C.; and wherein at least one primer of the set of release primers has a melting temperature greater than about 65° C. In some embodiments, the at least one primer of the set of capture primers has a melting temperature greater than about 72° C.; and wherein the at least one primer of the set of release primers has a melting temperature greater than about 72° C. In some embodiments, up to about 10% of the capture and/or release primers in the kit are high melting temperature and/or high GC content primers (e.g., pre-amplification primers, capture primers, release primers, amplification primers, etc.). In some embodiments, the at least one primer of the set of capture primers comprises one or more modified dNTPs; and wherein the at least one primer of the set of release primers comprises one or more modified dNTPs. In some embodiments, the kit further comprises at least one enhancer selected from the group consisting of betaine or a derivative or analog thereof, DMSO, a single stranded DNA binding protein, or a disaccharide.

An eleventh aspect of the present disclosure is a method of producing a capture primer extension complex comprising a target nucleic acid molecule and a capture primer, the method comprising: (a) hybridizing a capture primer to a portion of the target nucleic acid molecule in a library of nucleic acid molecules, where each of the nucleic acid molecules in the library of nucleic acid molecules has a first end comprising a first adapter and a second end comprising a second adapter; and (b) extending the hybridized capture primer with a first polymerase to producing the capture primer extension complex; wherein the capture primer has a melting temperature greater than 63° C. In some embodiments, the melting temperature of the capture primer is greater than 66° C. In some embodiments, the melting temperature of the capture primer is greater than 72° C. In some embodiments, the melting temperature of the capture primer is greater than 75° C.

In some embodiments, the capture primer hybridized to the target nucleic acid molecule is extended in a composition comprising at least one enhancer selected from the group consisting of betaine or a derivative or analog thereof, DMSO (or a solvent having similar chemical and/or physical properties, such as DMF), a single stranded DNA binding protein (such as a thermostable single-stranded DNA-binding protein, and a disaccharide (such as trehalose). In some embodiments, the at least one enhancer is betaine, and wherein the concentration of betaine ranges from between about 0.2 mM to about 0.8 mM. In some embodiments, the at least one enhancer is betaine, and wherein the concentration of betaine ranges from between about 0.3 mM to about 0.7 mM. In some embodiments, the at least one enhancer is betaine, and wherein the concentration of betaine ranges from between about 0.4 mM to about 0.6 mM.

In some embodiments, the capture primer hybridized to the target nucleic acid molecule is extended in a composition comprising one or more modified dNTPs. In some embodiments, the one or more modified dNTPs are modified dGTPs. In some embodiments, the modified dGTPs comprise 7-Deaza-2′-deoxyguanosine-5′-Triphosphates. In some embodiments, the one or more modified dNTPs are modified dATPs. In some embodiments, the modified dATPs comprise 2-Amino-2′ deoxyadenosine-5′-Triphosphates. In some embodiments, the capture primer hybridized to the target nucleic acid molecule is extended in a composition comprising betaine and one or more modified dNTPs.

In some embodiments, a concentration of betaine in the composition is about 0.5 mM. In some embodiments, the method further comprises capturing the capture primer extension complex. In some embodiments, the method further comprises hybridizing a release primer to the target nucleic acid. In some embodiments, the release primer is hybridized to the target nucleic acid in a composition comprising at least one enhancer. In some embodiments, the at least one enhancer is betaine. In some embodiments, the release primer is hybridized to the target nucleic acid in a composition comprising one or more dNTPs. In some embodiments, the release primer has a melting temperature greater than 63° C. In some embodiments, the melting temperature greater than 65° C. In some embodiments, the melting temperature greater than 72° C.

In some embodiments, the method further comprises extending the release primer hybridized to the target nucleic acid with a second polymerase.

A twelfth aspect of the present disclosure is a method of amplifying one or more nucleic acid molecules comprising: (i) obtaining a plurality of nucleic acid molecules; and (ii) performing a first amplification reaction in a first composition comprising the obtained plurality of nucleic acid molecules and a first set of primers, wherein at least one primer of the first set of primers has a melting temperature greater than 63° C. In some embodiments, each nucleic acid molecule of the obtained plurality of nucleic acid molecules comprises first and second adapters. In some embodiments, the first composition further comprises betaine. In some embodiments, wherein a concentration of betaine in the first composition is about 0.5 mM. In some embodiments, the method further comprises enriching the obtained plurality of nucleic acid molecules for one or more target nucleic acid molecules. In some embodiments, the method further comprises performing a second amplification reaction in a second composition comprising the one or more target nucleic acid molecules and a second set of primers, wherein at least one primer of the second set of primers has a melting temperature greater than 63° C. In some embodiments, the second composition further comprises betaine. In some embodiments, a concentration of betaine in the second composition is about 0.5 mM. In some embodiments, the obtained plurality of nucleic acid molecules is a target enriched library comprising a plurality of target nucleic acid molecules. In some embodiments, the method further comprises sequencing the amplified target enriched library.

A thirteenth aspect of the present disclosure is a method of amplifying one or more nucleic acid molecules comprising: (i) obtaining a plurality of nucleic acid molecules; (ii) performing a first amplification reaction in a first composition comprising the plurality of nucleic acid molecules, betaine, and a mixture of unmodified dNTPs and modified dNTPs. In some embodiments, a concentration of betaine in the first composition is about 0.5 mM. In some embodiments, at least one primer of the first set of primers has a high melting temperature and/or a high GC content. In some embodiments, each nucleic acid molecule of the obtained plurality of nucleic acid molecules comprises first and second adapters, and wherein the method further comprises enriching the obtained plurality of nucleic acid molecules for one or more target nucleic acid molecules. In some embodiments, the method further comprises performing a second amplification reaction in a second composition comprising the one or more target nucleic acid molecules, betaine, and a mixture of unmodified dNTPs and modified dNTPs. In some embodiments, the obtained plurality of nucleic acid molecules is a target enriched library comprising a plurality of target nucleic acid molecules. In some embodiments, further comprises sequencing the amplified target enriched library.

A fourteenth aspect of the present disclosure is a kit for the enrichment of at least one target nucleic acid in a library of nucleic acid molecules, the kit comprising: a first oligonucleotide complementary to a target nucleic acid in library of nucleic acid molecules, each of the nucleic acid molecules in the library of nucleic acid molecules having a first end comprising a first adapter and a second end comprising a second adapter; a second oligonucleotide complementary to the target nucleic acid; a first amplification primer; and a second amplification primer; and where at least one of the first oligonucleotide or the second oligonucleotide has a high melting temperature and/or a high GC content. In some embodiments, both the first and second oligonucleotides have a high melting temperature and/or a high GC content.

A fifteenth aspect of the present disclosure is a composition, comprising: a library of nucleic acid molecules comprising at least one target nucleic acid, each of the nucleic acid molecules in the library of nucleic acid molecules having a first end comprising a first adapter, a second end comprising a second adapter, and a region of interest intermediate the first adapter and the second adapter; an extended first oligonucleotide hybridized to the region of interest of the target nucleic acid, the extended first oligonucleotide including at least one capture moiety; a solid support bound to the at least one capture moiety; a second oligonucleotide hybridized to the target nucleic acid at a position 5′ to the first extended oligonucleotide; and a polymerase associated with a 3′ end of the second oligonucleotide; wherein at least one of the first oligonucleotide or the second oligonucleotide has a high melting temperature and/or a high GC content.

A sixteenth aspect of the present disclosure is a composition comprising a polymerase, one or more primers having no limits on melting temperature and/or GC content, a mixture of unmodified dNTPs and modified dNTPs, and at least one enhancer. In some embodiments, the at least one enhancer is betaine. In some embodiments, the composition further comprises one or more nucleic acid molecules.

A seventeenth aspect of the present disclosure is a composition consisting essentially of a polymerase, one or more primers having no limits on melting temperature and/or GC content, a mixture of unmodified dNTPs and modified dNTPs, and at least one enhancer. In some embodiments, the at least one enhancer is betaine. In some embodiments, the composition further comprises one or more nucleic acid molecules.

An eighteenth aspect of the present disclosure is a composition consisting of a polymerase, one or more primers having no limits on melting temperature and/or GC content, a mixture of unmodified dNTPs and modified dNTPs, and at least one enhancer. In some embodiments, the at least one enhancer is betaine. In some embodiments, the composition further comprises one or more nucleic acid molecules.

A nineteenth aspect of the present disclosure is a reaction tube comprising the composition of any one of the sixteenth, seventeenth, or eighteenth aspects of the present disclosure as noted above or as described herein.

A twentieth aspect of the present disclosure is a use of the composition of any one of the sixteenth, seventeenth, or eighteenth aspects of the present disclosure as noted above or as described herein in the amplification of one or more nucleic acid molecules, such as a library of nucleic acids or captured nucleic acid molecules.

A twenty-first aspect of the present disclosure is a method of amplifying one or more nucleic acid molecules, wherein the amplification is performed in the presence of any one of the compositions of the sixteenth, seventeenth, or eighteenth aspects of the present disclosure as noted above or as described herein. In some embodiments, a step of thermocycling is performed for a time period ranging from between about 25 minutes to about 35 minutes, such as for about 34 minutes.

### BRIEF DESCRIPTION OF THE FIGURES

The patent or application file contains at least one drawing executed in color. Copies of this patent or patent application publication with color drawing(s) will be provided by the Office upon request and payment of the necessary fee.

For a general understanding of the features of the disclosure, reference is made to the drawings. In the drawings, like reference numerals have been used throughout to identify identical elements.

FIG. 1 provides a flow chart illustrating an embodiment of a method for enrichment of at least one target nucleic acid molecule in a library of nucleic acid molecules according to the present disclosure. The steps illustrated may be performed in the presence of any of the compositions described herein.

FIG. 2A depicts that primer availability in target areas with high % GC is limited when primer % GC is restricted to between 20% and 80%, and primer Tm is restricted to between 57° C. and 63° C. Primer coverage over primary targets is incomplete with these restrictions to primer % GC and primer Tm.

FIG. 2B illustrates that by removing limitations on primer % GC content and primer Tm increases primer availability in target areas having high % GC content and improves primer coverage in these areas.

FIG. 2C shows that increasing the maximum primer Tm permitted during primer design decreases the number of target bases not covered by primers.

FIG. 3A illustrates samples enriched with primer test panels designed with primer Tm ranging from between about 57° C. to about 63° C. (Current Primer Database), from between about 57° C. to about 66° C. (Max Tm 66), from between about 57° C. to about 69° C. (Max Tm 69), from between about 57° C. to about 62° C. (Max Tm 72° C.), and from about 57° C. to “no Tm limit” (No Max Tm). Samples enriched with the “No Max Tm” test panel had the highest percent bases in 2-fold range, and percent panel exon region >=1000× which are measurements of sequencing coverage uniformity over targets.

FIG. 3B illustrates samples enriched with primer test panels designed with primer Tm from between about 57° C. to about 63° C. (Current Primer Database), from between about 57° C. to about 66° C. (Max Tm 66), from between about 57° C. to about 69° C. (Max Tm 69), from between about 57° C. to about 62° C. (Max Tm 72° C.), and from about 57° C. to no limit (No Max Tm). Normalized sequencing coverage in high % GC targets (65%, 75%, and 77%) is most improved in samples enriched with the No Max Tm test panel compared to samples enriched with the Current Primer Database

FIG. 4 depicts samples prepared into libraries with and without Betaine (PCR1+B, PCR1−B) and 7-deaza dGTP (PCR1+7, PCR1−7). Libraries were then used as input into HyperPETE with and without Betaine in capture extension (CapExt+B, CapExt−B). Yields for PCR1+B with PCR1−7 pre-capture input libraries were higher than PCR1−B with PCR1−7 pre-capture input libraries. Yields for PCR1+7 pre-capture input libraries were comparatively lower

FIG. 5 illustrates samples prepared into libraries with and without Betaine (PCR1+B, PCR1−B) and 7-deaza dGTP (PCR1+7, PCR1−7). Libraries were then used as input into HyperPETE with and without Betaine in capture extension (CapExt+B, CapExt−B). Compared to the PCR1−B samples, PCR1+B plus PCR1−7 samples had higher dedup depth and mean fragment length. Compared to CapExt−B samples, PCR1−7 with CapExt+B samples had higher percent bases in 2-fold and percent panel exon regions >=1000χ. PCR1+7 samples had higher percent bases in 2-fold and percent panel exon regions >=1000× regardless of the usage of Betaine in PCR1 or capture extension.

FIG. 6A illustrates samples prepared into libraries with and without 7-deaza dGTP (PCR1+7-deaza dGTP, PCR1−7-deaza dGTP). Libraries were then used as input into HyperPETE with and without Betaine in capture extension (Capture Extension+Betaine, Capture Extension-Betaine). Compared to PCR1−7 samples, normalized coverage across three high % GC targets is more uniform for PCR1+7 samples.

FIG. 6B shows samples prepared into libraries with and without 7-deaza dGTP (PCR1+7-deaza dGTP, PCR1−7-deaza dGTP). Libraries were then used as input into HyperPETE with and without Betaine in capture extension (Capture Extension+Betaine, Capture Extension−Betaine). For PCR1−7-deaza dGTP samples, normalized coverage across three high % GC targets is more uniform for Capture Extension+Betaine samples than for Capture Extension−Betaine samples.

FIG. 7 illustrates samples prepared into libraries with and without Betaine (PCR1+Betaine, PCR1−Betaine) and 7-deaza dGTP (PCR1+7-deaza dGTP, PCR1−7-deaza dGTP). Libraries were then used as input into HyperPETE with and without Betaine in capture extension (Capture Extension+Betaine, Capture Extension−Betaine). PCR1+7-deaza dGTP samples had the highest normalized coverage for high % GC targets (75%, 80%) followed by PCR1−7-deaza dGTP+Capture Extension+Betaine samples. PCR1+Betaine samples had higher coverage in low % GC (30%) targets compared to all other sample groups.

FIG. 8 depicts samples enriched using varying capture extension test programs detailed in Tables 3-6 herein and a control capture extension program following the HyperPETE instructions for use (IFU). All samples enriched with the test programs (Programs 1-4) had higher percent reads on target and dedup depth compared to IFU. Mean fragment length was much higher for IFU samples compared to test program samples. Program 3 samples had the highest percent bases in 2-fold range and percent panel exon region >=1000×.

FIG. 9 shows samples enriched using varying capture extension test programs detailed in Tables 3-6 herein and a control capture extension program following the HyperPETE instructions for use (IFU). Samples enriched using the test programs (Programs 1-4) had higher normalized coverage in high % GC targets (75%, 80%) compared to IFU samples with Program3 samples having the highest coverage.

FIG. 10 illustrates that out of all of the conditions tested in order to improve coverage in high % GC targets, normalized coverage in three high % GC targets (65%, 75%, 77%) was the highest, over 90% for all three targets, in samples made into libraries with 7-deaza dGTP and enriched with a panel designed with no primer % GC and max Tm limit (No Max Tm+PCR1 w/7-deaza dGTP).

FIG. 11 depicts sequencing quality control metrics.

FIG. 12 illustrates normalized position deduped coverage across high GC percentage regions of interest.

FIG. 13 illustrates normalized position deduped coverage by regions of interest.

FIG. 14 sets forth sequencing quality control metrics.

FIG. 15 illustrates normalized position deduped coverage across high GC percentage regions of interest.

FIG. 16 illustrates normalized position deduped coverage by regions of interest.

FIG. 17 provides sequencing quality control metrics for DMSO titration with a 1.7 Mb panel.

FIG. 18 illustrates DMSO titration with a 1.7 Mb panel, showing normalized position deduped coverage and high GC percentage.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

### Definitions

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “adapter” refers a nucleotide sequence that may be added to another sequence so as to import additional properties to that sequence. An adapter can be single- or double-stranded or may have both a single-stranded portion and a double-stranded portion.

As used herein “amplification” refers to a process in which a copy number increases. Amplification may be a process in which replication occurs repeatedly over time to form multiple copies of a template. Amplification can produce an exponential or linear increase in the number of copies as amplification proceeds. Exemplary amplification strategies include polymerase chain reaction (PCR), loop-mediated isothermal amplification (LAMP), rolling circle replication (RCA), cascade-RCA, nucleic acid-based amplification (NASBA), and the like. Also, amplification can utilize a linear or circular template. Amplification can be performed under any suitable temperature conditions, such as with thermal cycling or isothermally.

Furthermore, amplification can be performed in an amplification mixture (or reagent mixture), which is any composition capable of amplifying a nucleic acid target, if any, in the mixture. PCR amplification relies on repeated cycles of heating and cooling (i.e., thermal cycling) to achieve successive rounds of replication. PCR can be performed by thermal cycling between two or more temperature setpoints, such as a higher denaturation temperature and a lower annealing/extension temperature, or among three or more temperature setpoints, such as a higher denaturation temperature, a lower annealing temperature, and an intermediate extension temperature, among others. PCR can be performed with a thermostable polymerase, such as Taq DNA polymerase. PCR generally produces an exponential increase in the amount of a product amplicon over successive cycles. PCR is described, for example, in U.S. Pat. Nos. 4,683,202; 4,683,195; 4,000,159; 4,965,188; 5,176,995), the disclosures of each are hereby incorporated by reference herein in their entirety.

As used herein, the term “complementary” generally refers to the capability for precise pairing between two nucleotides. The term “complementary” refers to the ability to form favorable thermodynamic stability and specific pairing between the bases of two nucleotides at an appropriate temperature and ionic buffer conditions. Complementarity is achieved by distinct interactions between the nucleobases adenine, thymine (uracil in RNA), guanine and cytosine, where adenine pairs with thymine or uracil, and guanine pairs with cytosine. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence.

As used herein, the term “enrichment” refers to the process of increasing the relative abundance of a population of molecules, e.g., nucleic acid molecules, in a sample relative to the total amount of the molecules initially present in the sample before treatment. Thus, an enrichment step provides a percentage or fractional increase rather than directly increasing for example, the copy number of the nucleic acid sequences of interest as amplification methods, such as a polymerase chain reaction, would.

As used herein, the term “hybridize” refers to the base-pairing between different nucleic acid molecules consistent with their nucleotide sequences.

As used herein, the terms “modified dNTP” or “modified nucleoside triphosphate” refer to any molecule suitable for substituting one corresponding unmodified or classic dNTP. Such modified nucleotides must be able to undergo a base pair matching identical or similar to the unmodified dNTP it replaces. The modified nucleotide or modified dNTP may have a chemical structure similar to that of the corresponding nucleotide or dNTP but differ from the nucleotide or the dNTP in at least one atom or at least one bond type.

As used herein, the terms “nucleic acid” or “polynucleotide” (used interchangeably herein) refer to a polymeric form of nucleotides of any length, either deoxyribonucleotides or ribonucleotides, or analogs thereof. Polynucleotides may have any three-dimensional structure, and may perform any function, known or unknown. Unless specifically limited, the terms encompass nucleic acids or polynucleotides including known analogues of natural nucleotides that have similar binding properties as the reference nucleic acid and are metabolized in a manner similar to naturally occurring nucleotides. Non-limiting examples of polynucleotides include coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers.

A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

As used herein, the term “oligonucleotide,” refers to an oligomer of nucleotide or nucleoside monomer units wherein the oligomer optionally includes non-nucleotide monomer units, and/or other chemical groups attached at internal and/or external positions of the oligomer. The oligomer can be natural or synthetic and can include naturally-occurring oligonucleotides, or oligomers that include nucleosides with non-naturally-occurring (or modified) bases, sugar moieties, phosphodiester-analog linkages, and/or alternative monomer unit chiralities and isomeric structures (e.g., 5′- to 2′-linkage, L-nucleosides, α-anomer nucleosides, β-anomer nucleosides, locked nucleic acids (LNA), peptide nucleic acids (PNA)). By way of example, an oligonucleotide may be 10 to 20, 11 to 30, 31 to 40, 41 to 50, 51-60, 61 to 70, 71 to 80, 80 to 100, 100 to 150 or 150 to 200 nucleotides in length.

As used herein, the term “polymerase” refers to an enzyme that performs template-directed synthesis of polynucleotides. A DNA polymerase can add free nucleotides only to the 3′ end of the newly forming strand. This results in elongation of the newly forming strand in a 5′-3′ direction. No known DNA polymerase is able to begin a new chain (de novo). DNA polymerase can add a nucleotide only on to a pre-existing 3′-OH group, and, therefore, needs a primer at which it can add the first nucleotide. As used herein, the term “primer” refers to an oligonucleotide which binds to a specific region of a single-stranded template nucleic acid molecule and initiates nucleic acid synthesis via a polymerase-mediated enzymatic reaction, extending from the 3′ end of the primer and complementary to the sequence of the template molecule. PCR amplification primers can be referred to as ‘forward’ and ‘reverse’ primers, one of which is complementary to a nucleic acid strand and the other of which is complementary to the complement of that strand. Typically, a primer comprises fewer than about one hundred nucleotides and preferably comprises fewer than about 50 nucleotides. Exemplary primers range from about five to about 25 nucleotides. Primers can comprise, for example, RNA and/or DNA bases, as well as non-naturally occurring bases.

As used herein, the terms “unmodified dNTP” or “unmodified nucleoside triphosphate” refer to the four deoxyribonucleotide triphosphates dATP (deoxyadenosine triphosphate), dCTP (deoxycytidine triphosphate), dGTP (deoxyguanosine triphosphate) and dTTP (deoxythymidine triphosphate) that are normally used as building blocks in the synthesis of DNA.s

As used herein, the term “sequence,” when used in reference to a nucleic acid molecule, refers to the order of nucleotides (or bases) in the nucleic acid molecules. In cases, where different species of nucleotides are present in the nucleic acid molecule, the sequence includes an identification of the species of nucleotide (or base) at respective positions in the nucleic acid molecule. A sequence is a property of all or part of a nucleic acid molecule. The term can be used similarly to describe the order and positional identity of monomeric units in other polymers such as amino acid monomeric units of protein polymers.

As used herein, the term “sequencing” refers to the determination of the order and position of bases in a nucleic acid molecule. More particularly, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the terms “target” or “target sequence” refer to nucleic acid molecule sequences of interest, e.g., those which hybridize to oligonucleotide probes.

As used herein, the term “universal primer” refers to a primer that can hybridize to and support amplification of target polynucleotides having a shared complementary universal primer binding site. Similar, the term “universal primer pair” refers to a forward and reverse primer pair that can hybridize to and support PCR amplification of target polynucleotides having shared complementary forward and reverse universal primer binding sites. Such universal primer(s) and universal primer binding site(s) can allow single or double primer mediated universal amplification (e.g., universal PCR) of target polynucleotide regions of interest. The headings provided herein are for convenience only and do not interpret the scope or meaning of the disclosed embodiments.

### Overview

The present disclosure is directed to compositions that include one or more components which facilitate improving uniformity of coverage and/or reducing GC bias during one or more downstream sequencing operations. In some embodiments, the compositions described herein are useful in the preparation of target enriched samples and/or the amplification of such target enriched samples. Methods of preparing target enriched samples and/or the amplification of target enriched samples are disclosed in U.S. Patent Publication Nos. 2018/0016630 and 2020/0032244 and in International Application Nos. PCT/US2017/041748 and PCT/EP2018/08527, the disclosures of which are hereby incorporated herein in their entireties. In some embodiments, the compositions of the present disclosure may be used in one or more steps of preparing target enriched samples, including during pre-capture amplification, capture extension, and release primer hybridization and/or extension, and subsequently during post-capture amplification of the prepared target enriched sample.

The present disclosure is also directed to methods of preparing target enriched samples and methods of amplifying one or more targets in the prepared target enriched samples. In some embodiments, the steps of pre-capture, capture extension, release primer hybridization and extension, and/or post-capture amplification are conducted in the presence of one or more components which improve uniformity of coverage and/or reduce GC bias during one or more downstream sequencing operations. In some embodiments, the one or more components which increase uniformity of coverage and/or reduce GC bias during sequencing are enhancers including, but not limited to, betaine or its derivatives or analogs, DMSO (or like solvents having similar chemical and/or physical properties), single stranded DNA binding proteins, and disaccharides (such as trehalose). In other embodiments, the one or more components which increase uniformity of coverage and/or reduce GC bias during sequencing include high melting temperature primers or primers which have a high GC content. In yet other embodiments, the one or more components which increase uniformity of coverage and/or reduce GC bias are dNTP mixtures which include unmodified dNTPs and one or more modified dNTPs; or primers which include one or more modified dNTPS. Of course, any combination of any of these components may be used during any hybridization and/or extension reaction, as described in further detail herein.

The present disclosure also provides for methods of improving uniformity of coverage and/or reducing GC bias during sequencing by optimizing certain parameters of certain incubation steps utilized during the preparation of target enriched samples. For instance, the present disclosure provides methods of incubating samples at higher temperatures and/or for longer durations as compared with control incubation programs. These and other embodiments are described further herein.

### Compositions

As noted above, the present disclosure is directed to compositions for use in hybridization and/or extension reactions, where the compositions comprise one or more components which facilitate improving a uniformity of coverage and/or reducing GC bias during sequencing. In some embodiments, the one or more components which facilitate improving the uniformity of coverage during sequencing include (i) primers which have high GC content and/or high melting temperatures; (ii) primers which include one or modified dNTPs; (iii) enhancers; and/or (iv) dNTP mixtures which include unmodified and modified dNTPs. In some embodiments, enhancers include, but are not limited to, betaine, DMSO, single stranded DNA binding proteins, disaccharides (e.g., trehalose), and combinations thereof. Each of these components are described herein.

In some embodiments, the compositions of the present disclosure include both (a) primers which have high GC content and/or high melting temperatures; and (b) one or more enhancers. In other embodiments, the compositions of the present disclosure include both (a) primers which have high GC content and/or high melting temperatures; and (b) primers which include one or more modified dNTPs. In yet other embodiments, the compositions of the present disclosure include both (a) primers which include one or modified dNTPs; and (b) one or more enhancers. In further embodiments, the compositions of the present disclosure include both (a) one or more enhancers; and (b) mixtures of modified and unmodified dNTPs. In even further embodiments, the compositions of the present disclosure include (a) primers which have high GC content and/or high melting temperatures; (b) primers which include one or modified dNTPs; and (c) one or more enhancers. In yet even further embodiments, the compositions of the present disclosure include (a) primers which have high GC content and/or high melting temperatures; (b) primers which include one or modified dNTPs; and (c) mixtures of unmodified dNTPs and modified dNTPs.

In some embodiments, the compositions include one or more primers; one or more polymerases; deoxynucleoside triphosphates (dNTPs) (e.g., unmodified dNTPs or mixtures of unmodified and modified dNTPs); and at least one enhancer. In other embodiments, the compositions include one or more primers, where at least one primer of the one or more primers has a high melting temperature (or a high GC content) and/or includes one or more modified dNTPs; one or more polymerases; dNTPs (e.g., unmodified dNTPs or mixtures of unmodified and modified dNTPs); and optionally at least one enhancer. In some embodiments, the compositions further include one or more input nucleic acid molecules; one or more buffers; one or more divalent cations; one or more cofactors; and/or one or more polyols.

In some embodiments, the compositions include one or more primers; one or more nucleic acid molecules; and at least one enhancer. In other embodiments, the compositions include one or more primers, where at least one primer of the one or more primers has a high melting temperature (or high GC content) and/or includes one or more modified dNTPs (e.g., unmodified dNTPs or mixtures of unmodified and modified dNTPs); one or more nucleic acid molecules; and optionally one or more enhancers. In some embodiments, the compositions further include one or more polymerases; dNTPs; one or more buffers; one or more divalent cations; one or more cofactors; and/or one or more polyols.

### Primers

Primers, including any of the pre-capture primers (e.g., pre-capture forward and pre-capture reverse primers), capture primers, release primers, and/or post-capture amplification primers described herein, may be synthesized to include at least one modified dNTP, including any of the modified dNTPs described herein. Likewise, primers, including any of the pre-capture primers (e.g., pre-capture forward and reverse primers), capture primers, release primers, and/or post-capture amplification primers described herein, may be synthesized to include a high GC content and/or to have a high melting temperature. In addition, primers, including any of the pre-capture primers (e.g., pre-capture forward and reverse primers), capture primers, release primers, and/or post-capture amplification primers described herein, may be synthesized to include (i) a high GC content and/or to have a high melting temperature; and (ii) one or more modified dNTPs.

In some embodiments, the compositions of the present disclosure include one or more primers, wherein at least one primer of the one or more primers has a high GC content and/or a high melting temperature. By “high GC content” it is meant that the at least one primer of the one or more primers has a GC content of greater than about 70%. For instance, a high GC content primer may have a GC content of at least about 75%, at least about 80%, at least about 81%, a GC content of at least about 82%, a GC content of at least about 83%, a GC content of at least about 84%, a GC content of at least about 85%, a GC content of at least about 86%, a GC content of at least about 87%, a GC content of at least about 88% a GC content of at least about 89%, a GC content of at least about 90%, a GC content of at least about 91%, a GC content of at least about 92%, a GC content of at least about 93%, a GC content of at least about 94%, a GC content of at least about 95%, a GC content of at least about 96%, a GC content of at least about 97%, a GC content of at least about 98%, or a GC content of at least about 99%. In some embodiments, high GC primers includes no limitation on GC content.

By “high melting temperature” it is meant that at least one primer of the one or more primers has a melting temperature of greater than 63° C. In some embodiments, the at least one primer of the one or more primers has a melting temperature greater than about 64° C., greater than about 65° C., greater than about 66° C., greater than about 67° C., greater than about 68° C., greater than about 69° C., greater than about 70° C., greater than about 71° C., greater than about 72° C., greater than about 73° C., greater than about 74° C., greater than about 75° C., greater than about 76° C., greater than about 77° C., greater than about 78° C., greater than about 79° C., greater than about 80° C., greater than about 81° C., greater than about 82° C., greater than about 83° C., greater than about 84° C., greater than about 85° C., greater than about 86° C., greater than about 87° C., greater than about 88° C., greater than about 89° C., greater than about 90° C., greater than about 91° C., least about 92° C., greater than about 93° C., greater than about 94° C., greater than about 95° C., greater than about 96° C., greater than about 97° C., greater than about 98° C., greater than about 99° C., greater than about 100° C., etc.

In some embodiments, primers included within any set of primers may have melting temperatures ranging from between about 57° C. to about 63° C., from between about 57° C. to about 64° C., from between about 57° C. to about 65° C., from between about 57° C. to about 66° C., from between about 57° C. to about 67° C., from between about 57° C. to about 68° C., from between about 57° C. to about 69° C., from between about 57° C. to about 70° C., from between about 57° C. to about 71° C., from between about 57° C. to about 72° C., from between about 57° C. to about 73° C., from between about 57° C. to about 74° C., from between about 57° C. to about 75° C., from between about 57° C. to about 76° C., from between about 57° C. to about 77° C., from between about 57° C. to about 78° C., from between about 57° C. to about 79° C., from between about 57° C. to about 80° C., from between about 57° C. to about 81° C., from between about 57° C. to about 82° C., from between about 57° C. to about 83° C., from between about 57° C. to about 84° C., from between about 57° C. to about 85° C., from between about 57° C. to about 86° C., from between about 57° C. to about 87° C., from between about 57° C. to about 88° C., from between about 57° C. to about 89° C., from between about 57° C. to about 90° C., from between about 57° C. to about 91° C., from between about 57° C. to about 92° C., from between about 57° C. to about 93° C., from between about 57° C. to about 94° C., from between about 57° C. to about 95° C., from between about 57° C. to about 96° C., from between about 57° C. to about 97° C., from between about 57° C. to about 98° C., from between about 57° C. to about 99° C., from between about 57° C. to about 100° C., etc. In some embodiments, at least one primer in the set of primers having any of the aforementioned temperature ranges, has a melting temperature greater than 63° C. Of course, the skilled artisan will appreciate that any of the primers within the set of primers (including the at least one primer that has a melting temperature greater than 63° C.) may include one or more modified dNTPs, e.g. one or more modified dGTPs or dATPs.

In some embodiments, up to about 1% of the primers within any set of primers (in any composition) have a high GC content and/or a high melting temperature. In some embodiments, up to about 2% of the primers within any set of primers have a high GC content and/or a high melting temperature. In some embodiments, up to about 3% of the primers within any set of primers have a high GC content and/or a high melting temperature. In some embodiments, up to about 4% of the primers within any set of primers have a high GC content and/or a high melting temperature. In some embodiments, up to about 5% of the primers within any set of primers have a high GC content and/or a high melting temperature. In some embodiments, up to about 6% of the primers within any set of primers have a high GC content and/or a high melting temperature. In some embodiments, up to about 7% of the primers within any set of primers have a high GC content and/or a high melting temperature. In some embodiments, up to about 8% of the primers within any set of primers have a high GC content and/or a high melting temperature. In some embodiments, up to about 9% of the primers within any set of primers have a high GC content and/or a high melting temperature. In some embodiments, up to about 10% of the primers within any set of primers have a high GC content and/or a high melting temperature. In some embodiments, up to about 15% of the primers within any set of primers have a high GC content and/or a high melting temperature. In some embodiments, up to about 20% of the primers within any set of primers have a high GC content and/or a high melting temperature. In some embodiments, up to about 25% of the primers within any set of primers have a high GC content and/or a high melting temperature. In some embodiments, up to about 30% of the primers within any set of primers have a high GC content and/or a high melting temperature.

In some embodiments, two or more modified dNTPs may be used in any of the primers within any of the compositions described herein. In some embodiments, the primers may include two or more modified dNTPs that are the same. In other embodiments, the primers may include two or more modified dNTPs that are different. In some embodiments, forward and reverse primers may include the same or different dNTP analogs. In some embodiments, up to about 5% of the nucleoside triphosphates included within any primer are modified dNTPs. In some embodiments, up to about 10% of the nucleoside triphosphates included within any primer are modified dNTPs. In other embodiments, up to about 15% of the nucleoside triphosphates included within any primer are modified dNTPs. In yet other embodiments, up to about 20% of the nucleoside triphosphates included within any primer are modified dNTPs. In further embodiments, up to about 25% of the nucleoside triphosphates included within any primer are modified dNTPs. In even further embodiments, up to about 30% of the nucleoside triphosphates included within any primer are modified dNTPs. In yet even further embodiments, up to about 35% of the nucleoside triphosphates included within any primer are modified dNTPs.

In some embodiments, at least one primer in any composition may have both (i) a high GC content and/or a high melt temperature; and (ii) may also include one or more modified dNTPs, e.g., two or more modified dNTPs, three or more modified dNTPs, four or more modified dNTPs, six of more modified dTNPS, seven or more modified dNTPs, etc. In other embodiments, a plurality of primers included within any composition may have both (i) a high GC content and/or a high melt temperature; and (ii) may also include one or more modified dNTPs, e.g., two or more modified dNTPs, three or more modified dNTPs, four or more modified dNTPs, six of more modified dTNPs, seven or more modified dNTPs, etc.

In some embodiments, the primers are target-specific primers that have a sequence that is complementary to a sequence of a target nucleic acid. One example of a target-specific primer is a gene-specific primer designed to hybridize to or nearby (e.g., upstream of, or 5′ to) a gene (e.g., cDNA, genomic DNA) of interest. The target nucleic acid can be RNA, DNA, or a combination thereof.

dNTPs

In some embodiments, the compositions of the present disclosure include one or more dNTPs, including unmodified dNTPs and/or modified dNTPs. In some embodiments, dNTPs are selected from the group consisting of unmodified dNTPs (dCTP, dATP, dGTP, dTTP, and dUTP). In other embodiments, one or more modified dNTPs are used instead of or in addition to unmodified dNTPs.

Modified dNTPs include, but are not limited to, 5-aminoallyl-2′-dCTP, 5-(3-aminoallyl)-2′-deoxycytidine-5′-triphosphate (5-aminoallyl-2′-dCTP), 2′-deoxycytidine-5′-O-(1-thiotriphosphate) ((1-thio)-2′-dCTP), 2′-deoxy-5-methylcytidine 5′-triphosphate (5-methyl-2′-dCTP), 2-thio-2′-deoxycytidine-5′-triphosphate (2-thio-2′-dCTP), 5-iodo-2′-deoxycytidine-5′-triphosphate (5-iodo-2′-dCTP), 2-amino-2′-deoxyadenosine-5′-triphosphate (2-amino-2′-dATP), 2-thiothymidine-5′-triphosphate (thio-TTP), 5-propynyl-2′-deoxycytidine-5′-triphosphate (5-propynyl-2′-dCTP), N4-methyl-2′-deoxycytidine-5′-triphosphate (N4-methyl-2′-dCTP), 7-deaza-2′-deoxyadenosine-5′-triphosphate (7-deaza-2′-dATP), 2′-deoxyguanosine-5′-O-(1-thiotriphosphate) ((1-thio)-2′-dGTP), 2′-deoxyadenosine-5′-O-(1-thiotriphosphate) ((1-thio)-2′-dATP), 5-bromo-2′-deoxycytidine-5′-triphosphate (5-bromo-2′-dCTP), and 7-deaza-2′-deoxyguanosine-5′-triphosphate (7-deaza-dGTP). In some embodiments, the modified dNTP is selected from one of (1-thio)-2′-dCTP, N4-methyl-2′-dCTP, 7-deaza-2′-dATP, 2-amino-2′-dATP, (1-thio)-2′dGTP, and 7-deaza-dGTP. In other embodiments, the modified dNTP is selected from 2-amino-2′-dATP and 7-deaza-dGTP.

In some embodiments, the compositions of the present disclosure in a mixture of unmodified dNTPs and modified dNTPs. In some embodiments, a ratio between the modified dNTP and the corresponding unmodified dNTP may range from about 1:3, about 1:2, about 1:1, about 2:1, and about 3:1. In some embodiments, a ratio of unmodified dNTPs to modified dNTPs is about 1:1.

In some embodiments, a composition may comprise between about 0.1 mM and about 0.7 mM of unmodified dNTPs; and may further comprise one or more modified dNTPs. In other embodiments, a composition may comprise between about 0.1 mM and about 0.6 mM of unmodified dNTPs; and may further comprise one or more modified dNTPs. In yet other embodiments, a composition may comprise between about 0.2 mM and about 0.5 mM of unmodified dNTPs; and may further comprise one or more modified dNTPs.

In some embodiments, a composition may comprise between about 0.1 mM and about 0.7 mM of unmodified dNTPs; and may further comprise between about 0.1 mM to about 0.7 mM of one or more modified dNTPs. In other embodiments, a composition may comprise between about 0.1 mM and about 0.6 mM of unmodified dNTPs; and may further comprise between about 0.1 mM to about 0.6 mM of one or more modified dNTPs. In yet other embodiments, a composition may comprise between about 0.2 mM and about 0.5 mM of unmodified dNTPs; and may further comprise between about 0.2 mM to about 0.5 mM of one or more modified dNTPs. In further embodiments, a composition may comprise between about 0.2 mM and about 0.4 mM of unmodified dNTPs; and may further comprise between about 0.2 mM to about 0.4 mM of one or more modified dNTPs. In even further embodiments, a composition may comprise about 0.3 mM of unmodified dNTPs; and may further comprise between about 0.2 mM to about 0.5 mM of one or more modified dNTPs. In even further embodiments, a composition may comprise about 0.3 mM of unmodified dNTPs; and may further comprise about 0.3 mM of one or more modified dNTPs.

In some embodiments, a composition may comprise between about 0.1 mM and about 0.7 mM of unmodified dNTPs; and may further comprise between about 0.1 mM to about 0.8 mM of one or more modified dNTPs. In other embodiments, a composition may comprise between about 0.1 mM and about 0.6 mM of unmodified dNTPs; and may further comprise between about 0.2 mM to about 0.7 mM of one or more modified dNTPs. In yet other embodiments, a composition may comprise between about 0.2 mM and about 0.5 mM of unmodified dNTPs; and may further comprise between about 0.2 mM to about 0.6 mM of one or more modified dNTPs.

### Enhancers

The compositions of the present disclosure may optionally include one or more enhancers selected from betaine (or any derivatives or analogs thereof), DMSO (or solvents having similar chemical and/or physical properties, such as DMF), single stranded DNA binding proteins, disaccharides (e.g., trehalose, sucrose, lactose, maltose, etc.), and combinations thereof. In some embodiments, the compositions of the present disclosure may include one enhancer. In other embodiments, the compositions of the present disclosure may include two enhancers. In yet other embodiments, the compositions of the present disclosure may include three enhancers. In further embodiments, the compositions of the present disclosure may include four enhancers.

In some embodiments, the enhancer is betaine (or a derivative or analog thereof) and a concentration of betaine in any composition ranges from between about 0.05 mM to about 1 mM. In other embodiments, the enhancer is betaine and a concentration of betaine in any composition ranges from between about 0.1 mM to about 0.9 mM. In other embodiments, the enhancer is betaine and a concentration of betaine in any composition ranges from between about 0.2 mM to about 0.8 mM. In other embodiments, the enhancer is betaine and a concentration of betaine in any composition ranges from between about 0.3 mM to about 0.7 mM. In other embodiments, the enhancer is betaine and a concentration of betaine in any composition ranges from between about 0.4 mM to about 0.6 mM. In some embodiments, the enhancer is betaine and a concentration of betaine in any composition is at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, etc.

In some embodiments, the enhancer is DMSO and the DMSO may be present in any composition in an amount up to about 15% (v/v). In some embodiments, the enhancer is DMSO and the DMSO may be present in any composition in an amount up to about 10% (v/v). In some embodiments, the enhancer is DMSO and the DMSO may be present in any composition in an amount up to about 9% (v/v). In some embodiments, the enhancer is DMSO and the DMSO may be present in any composition in an amount up to about 8% (v/v). In some embodiments, the enhancer is DMSO and the DMSO may be present in any composition in an amount up to about 7% (v/v). In some embodiments, the enhancer is DMSO and the DMSO may be present in any composition in an amount up to about 6% (v/v). In some embodiments, the enhancer is DMSO and the DMSO may be present in any composition in an amount up to about 5% (v/v). In some embodiments, the enhancer is DMSO and the DMSO may be present in any composition in an amount up to about 4% (v/v). In some embodiments, the enhancer is DMSO and the DMSO may be present in any composition in an amount up to about 3% (v/v). In some embodiments, the enhancer is DMSO and the DMSO may be present in any composition in an amount up to about 2% (v/v). In some embodiments, the enhancer is DMSO and the DMSO may be present in any composition in an amount up to about 1 (v/v).

In some embodiments, the enhancer is DMSO and an amount of DSMO in any composition ranges from between about 0.1% (v/v) to about 10% (v/v) of the composition. In other embodiments, the enhancer is DMSO and an amount of DSMO in any composition ranges from between about 0.5% (v/v) to about 10% (v/v) of the composition. In other embodiments, the enhancer is DMSO and an amount of DSMO in any composition ranges from between about 1% (v/v) to about 10% (v/v) of the composition. In other embodiments, the enhancer is DMSO and an amount of DSMO in any composition ranges from between about 2% (v/v) to about 8% (v/v) of the composition. In other embodiments, the enhancer is DMSO and an amount of DSMO in any composition ranges from between about 3% (v/v) to about 7% (v/v) of the composition.

In some embodiments, the single-stranded DNA-binding protein is a thermostable binding protein, such as some derived from a hyperthermophilic microorganism (e.g., ET SSB, available from New England BioLabs, Inc.). In other embodiments, the single-stranded DNA-binding protein is derived from Escherichia coli, Drosophila or Xenopus, a T4 bacteriophage-derived 32, 41, 44, 45 or 61 gene protein or an RPA protein in a eukaryote. A non-limiting example of a single-stranded DNA binding protein is the gene 2.5 protein of bacteriophage T7 and equivalent such T7-type phage proteins or other proteins. Examples of T7-type phages include T7, T3, ΦI, ΦII, H, W31, gh-1, Y, A1122 and SP6. In some embodiments, a single-stranded DNA-binding protein may be present in an amount ranging from between 0 ng/μL to about 50 ng/μL. In some embodiments, a single-stranded DNA-binding protein may be present in an amount ranging from between Ing/μL to about 50 ng/μL. In some embodiments, a single-stranded DNA-binding protein may be present in an amount ranging from between 5 ng/μL to about 50 ng/μL. In some embodiments, a single-stranded DNA-binding protein may be present in an amount ranging from between 10 ng/μL to about 50 ng/μL. In some embodiments, a single-stranded DNA-binding protein may be present in an amount ranging from between 5 ng/μL to about 40 ng/μL.

In some embodiments, the compositions of the present disclosure includes a disaccharide, such as trehalose. In some embodiments, the disaccharide is present at a concentration ranging from between about 0M to about 1M. In some embodiments, the disaccharide is present at a concentration ranging from between about 0.1M to about 1M. In some embodiments, the disaccharide is present at a concentration ranging from between about 0.1M to about 0.8M. In some embodiments, the disaccharide is present at a concentration ranging from between about 0.2M to about 0.8M.

### Polymerases

In some embodiments, the compositions of the present disclosure include one or more polymerases. Non-limiting examples of polymerases include prokaryotic DNA polymerases (e.g., Pol I, Pol II, Pol III, Pol IV, and Pol V), eukaryotic DNA polymerase, archaeal DNA polymerase, telomerase, reverse transcriptase, and RNA polymerase. Reverse transcriptase is an RNA-dependent DNA polymerase which synthesizes DNA from an RNA template. The reverse transcriptase family contain both DNA polymerase functionality and RNase H functionality, which degrades RNA base-paired to DNA. RNA polymerase is an enzyme that synthesizes RNA using DNA as a template during the process of gene transcription. RNA polymerase polymerizes ribonucleotides at the 3′ end of an RNA transcript.

In some embodiments, a polymerase from the following may be used in a polymerase-mediated primer extension, end-modification (e.g., terminal transferase, degradation, or polishing), or amplification reaction: archaea (e.g., Thermococcus litoralis (Vent, GenBank: AAA72101), Pyrococcus furiosus (Pfu, GenBank: D12983, BAA02362), Pyrococcus woesii, Pyrococcus GB-D (Deep Vent, GenBank: AAA67131), Thermococcus kodakaraensis KODI (KOD, GenBank: BD175553, BAA06142; Thermococcus sp. strain KOD (Pfx, GenBank: AAE68738)), Thermococcus gorgonarius (Tgo, Pdb: 4699806), Sulfolobus solataricus (GenBank: NC002754, P26811), Aeropyrum pernix (GenBank: BAA81109), Archaeglobus fulgidus (GenBank: 029753), Pyrobaculum aerophilum (GenBank: AAL63952), Pyrodictium occultum (GenBank: BAA07579, BAA07580), Thermococcus 9 degree Nm (GenBank: AAA88769, Q56366), Thermococcus fumicolans (GenBank: CAA93738, P74918), Thermococcus hydrothermalis (GenBank: CAC18555), Thermococcus sp. GE8 (GenBank: CAC12850), Thermococcus sp. JDF-3 (GenBank: AX135456; WO0132887), Thermococcus sp. TY (GenBank: CAA73475), Pyrococcus abyssi (GenBank: P77916), Pyrococcus glycovorans (GenBank: CAC12849), Pyrococcus horikoshii (GenBank: NP 143776), Pyrococcus sp. GE23 (GenBank: CAA90887), Pyrococcus sp. ST700 (GenBank: CAC 12847), Thermococcus pacificus (GenBank: AX411312.1), Thermococcus zilligii (GenBank: DQ3366890), Thermococcus aggregans, Thermococcus barossii, Thermococcus celer (GenBank: DD259850.1), Thermococcus profundus (GenBank: E14137), Thermococcus siculi (GenBank: DD259857.1), Thermococcus thioreducens, Thermococcus onnurineus NA1, Sulfolobus acidocaldarium, Sulfolobus tokodaii, Pyrobaculum calidifontis, Pyrobaculum islandicum (GenBank: AAF27815), Methanococcus jannaschii (GenBank: Q58295), Desulforococcus species TOK, Desulforococcus, Pyrolobus, Pyrodictium, Staphylothermus, Vulcanisaetta, Methanococcus (GenBank: P52025) and other archaeal B polymerases, such as GenBank AAC62712, P956901, BAAA07579)), thermophilic bacteria Thermus species (e.g., flavus, ruber, thermophilus, lacteus, rubens, aquaticus), Bacillus stearothermophilus, Thermotoga maritima, Methanothermus fervidus, KOD polymerase, TNA1 polymerase, Thermococcus sp. 9 degrees N-7, T4, T7, phi29, Pyrococcus furiosus, P. abyssi, T. gorgonarius, T. litoralis, T. zilligii, T. sp. GT, P. sp. GB-D, KOD, Pfu, T. gorgonarius, T. zilligii, T. litoralis and Thermococcus sp. 9N-7 polymerases.

In some embodiments, the compositions of the present disclosure includes one or more thermostable polymerases. As used herein, the term “thermostable polymerase” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. The heating conditions necessary for nucleic acid denaturation are well known in the art and are exemplified in, e.g., U.S. Pat. Nos. 4,683,202, 4,683,195, and 4,965,188, which are incorporated herein by reference. As used herein, a thermostable polymerase is suitable for use in a temperature cycling reaction such as the polymerase chain reaction (“PCR”), a primer extension reaction, or an end-modification (e.g., terminal transferase, degradation, or polishing) reaction. Irreversible denaturation for purposes herein refers to permanent and complete loss of enzymatic activity. For a thermostable polymerase, enzymatic activity refers to the catalysis of the combination of the nucleotides in the proper manner to form polynucleotide extension products that are complementary to a template nucleic acid strand. Thermostable DNA polymerases include DNA polymerases from Thermotoga maritima, Thermus aquaticus, Thermus thermophilus, Thermus flavus, Thermus filiformis, Thermus species sps17, Thermus species Z05, Thermus caldophilus, Bacillus caldotenax, Thermotoga neopolitana, Thermosipho africanus, and other thermostable DNA polymerases disclosed above.

One example of a polymerase is a Taq or Taq-derived polymerase (e.g., KAPA 2G polymerase from KAPA BIOSYSTEMS). Another example polymerase is a B-family DNA polymerase (e.g., KAPA HIFI polymerase from KAPA BIOSYSTEMS).

### Input Nucleic Acid Molecules

In some embodiments, input nucleic acid molecules or a library of input nucleic acid molecules are included in the compositions of the present disclosure. In some embodiments, the nucleic acid molecules within the obtained nucleic acid library are selected from DNA molecules, RNA molecules, genomic DNA molecules, cDNA molecules, mRNA molecules, rRNA molecules, mtDNA, siRNA molecules, or any combination thereof. In some embodiments, the plurality of nucleic acid molecules comprises single stranded polynucleotides. In some embodiments, the plurality of nucleic acid molecules is derived from a tissue sample, such as a tissue sample derived from a mammalian subject. In other embodiments, the plurality of nucleic acid molecules is derived from a formalin-fixed paraffin-embedded sample or cytology sample, such as a cytology sample derived from a mammalian subject.

In some embodiments, an obtained nucleic acid library includes a plurality of target nucleic acid molecules and/or a plurality of non-target nucleic acid molecules. In some embodiments, the non-target nucleic acid molecules are in high abundance as compared with the target nucleic acid molecules within the nucleic acid library. In some embodiments, the non-target nucleic acid molecules represent at least 70% of the nucleic acid molecules in the obtained nucleic acid library. In other embodiments, the non-target nucleic acid molecules represent at least 75% of the nucleic acid molecules in the obtained nucleic acid library. In yet other embodiments, the non-target nucleic acid molecules represent at least 80% of the nucleic acid molecules in the obtained nucleic acid library. In further embodiments, the non-target nucleic acid molecules represent at least 85% of the nucleic acid molecules in the obtained nucleic acid library. In yet further embodiments, the non-target nucleic acid molecules represent at least 90% of the nucleic acid molecules in the obtained nucleic acid library. In even further embodiments, the non-target nucleic acid molecules represent at least 95% of the nucleic acid molecules in the obtained nucleic acid library. In some embodiments, the non-target nucleic acid molecules represent at least 96% of the nucleic acid molecules in the obtained nucleic acid library. In some embodiments, the non-target nucleic acid molecules represent at least 97% of the nucleic acid molecules in the obtained nucleic acid library. In some embodiments, the non-target nucleic acid molecules represent at least 98% of the nucleic acid molecules in the obtained nucleic acid library. In some embodiments, the non-target nucleic acid molecules represent at least 99% of the nucleic acid molecules in the obtained nucleic acid library.

In some embodiments, the nucleic acid molecules within any library include ligated first and second adapters. For instance, in some embodiments, nucleic acid fragments are first prepared from a biological sample, e.g., a tissue sample and/or a cytology sample. The DNA sequencing library may be constructed from genomic DNA for genome analysis, or from cDNA prepared from RNA or mRNA for transcriptome analysis, and it may be constructed from the DNA or cDNA of any species of organism from which these nucleic acids can be extracted. In some embodiments, the obtained sample is sheared into fragments to provide a population of nucleic acid fragments. In some embodiments, shearing of the obtained genomic sample is effectuated using mechanical (e.g., nebulization or sonication) and/or enzymatic fragmentation (e.g., restriction endonucleases). In some embodiments, the generated nucleic acid fragments are randomly sized. In some embodiments, the generated nucleic acid fragments have a length which are less than about one thousand base pairs. In other embodiments, the generated nucleic acid fragments comprise sequence fragments having a sequence size ranging from between about one hundred to about one thousand base pairs in length. In yet other embodiments, the generated nucleic acid fragments comprise sequence fragments having a sequence size ranging from between about five hundred to about 750 base pairs in length.

In some embodiments, adapters are then added via a ligation reaction to the population of nucleic acid molecules. In some embodiments, the adapters include one or more barcode sequences. Methods of ligating adapters to a nucleic acid molecule are described in U.S. Patent Publication Nos. 2017/0037459, 2018/0334709, 2018/0016630 and in PCT Publication No. WO2017021449, the disclosures of which are hereby incorporated by reference herein in their entireties.

In some embodiments, the input nucleic acid molecules include a complex comprising a target nucleic acid molecule to which at least one primer is hybridized. For instance, an input nucleic acid molecule may comprise a complex of a target nucleic acid to which a capture primer is hybridized. In some embodiments, the capture primer of the complex may be extended.

In other embodiments, the input nucleic acid molecules include a complex comprising a target nucleic acid molecule to which first and second primers are hybridized (including primers which have high melting temperatures and/or no restriction on GC content). For instance, an input nucleic acid molecule may comprise a complex of a target nucleic acid molecule and an extended capture primer; and where the complex further includes a release primer.

In yet other embodiments, an input nucleic acid molecule may comprise a complex of a target nucleic acid molecule and an extended-release primer.

In some embodiments, the input nucleic acid molecules are target enriched, i.e., including a plurality of target nucleic acid molecules and being devoid of or substantially devoid non-target nucleic acid molecules.

### Buffers

The compositions of the present disclosure may include one or more buffers. Non-liming examples of buffers include citric acid, potassium dihydrogen phosphate, boric acid, diethyl barbituric acid, piperazine-N,N′-bis(2-ethanesulfonic acid), dimethylarsinic acid, 2-(N-morpholino)ethanesulfonic acid, tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), and combinations thereof. In other embodiments, the buffer may be comprised of tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof. Additional wash solutions, transfer solutions, acid solutions, and alkaline solutions are described in United States Patent Application Publication No. 2016/0282374, the disclosure of which is hereby incorporated by reference herein in its entirety.

### Divalent Cations

In some embodiments, the compositions of the present disclosure include one or more divalent cations. In some embodiments, the divalent cation is selected from Co²⁺, Mn²⁺, Mg²⁺, Cd²⁺, and Ca²⁺.

In some embodiments, any of the compositions of the present disclosure may include a concentration of divalent cation which is at least about 0.01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc., In some embodiments, any of the compositions of the present disclosure may have a concentration of a CoCl₂ of at least about 0.01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc. In some embodiments, any of the of the present disclosure may have a concentration of MnCl₂ which is at least about 0.01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc. In some embodiments, any of the compositions of the present disclosure may have a concentration of MgCl₂ which is at least about 0.01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc. In some embodiments, any of the compositions of the present disclosure may have a concentration of CdCl₂ which is at least about 0 . . . 01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc. In some embodiments, any of the compositions of the present disclosure may have a concentration of CaCl₂) which is at least about 0.01 mM, about 0.02 mM, about 0.05 mM, about 0.1 mM, about 0.2 mM, about 0.5 mM, about 1 mM, about 2 mM, about 5 mM, about 8 mM, about 10 mM, about 12 mM, about 15 mM, about 20 mM, about 30 mM, about 40 mM, about 50 mM, about 60 mM, about 70 mM, about 80 mM, about 90 mM, about 100 mM, etc.

### Polyols

In some embodiments, any of the compositions of the present disclosure may include one or more polyols. Suitable polyols include 1,2-ethanediol, 1,2-propanediol, 1,3-propanediol, 2-methyl-1,3-propanediol, 2-methyl-1,2-propanediol, 2,2-dimethyl-1,3-propanediol, 2,2-diethyl-1,3-propanediol, 2-methyl-2-propyl-1,3-propanediol, 2-butyl-2-ethyl-1,3-propanediol, dihydroxyacetone, 2,2-dibutyl-1,3-propanediol, 3-methoxy-1,3-propanediol, 3-methoxy-1,2-propanediol, 3-methoxy-2,3-propanediol, 2-methoxymethyl-1,3-propanediol, 3-ethoxy-1,3-propanediol, 3-ethoxy-1,2-propanediol, 3-ethoxy-2,3-propanediol, 3-allyloxy-1,2-propanediol, 1,2-butanediol, 1,3-butanediol, 1,4-butanediol, 2,3-butanediol, 2,3-dimethyl-2,3-butanediol, 3,3-dimethyl-1,2-butanediol, 1,2-pentanediol, 1,3-pentanediol, 1,4-pentanediol, 1,5-pentanediol, 2,3-pentanediol, 2,4-pentanediol, 2-methyl-2,4-pentanediol, 2,4-dimethyl-2,4-pentanediol, 2,2,4-trimethyl-1,3-pentanediol, 1,2-hexanediol, 1,3-hexanediol, 1,4-hexanediol, 1,5-hexanediol, 1,6-hexanediol, 2,3-hexanediol, 2,4-hexanediol, 2,5-hexanediol, 3,4-hexanediol, 2,5-dimethyl-2,5-hexanediol, 2-ethyl-1,3-hexanediol, 1,2-heptanediol, 1,3-heptanediol, 1,4-heptanediol, 1,5-heptanediol, 1,6-heptanediol, 1,7-heptanediol, 1,8-octanediol, 1,2-octanediol, 1,3-octanediol 1,4-octanediol, 1,5-octanediol, 1,6-octanediol, 1,7-octanediol, 1,2-nonadiol, 1,9-nonadiol, 1,10-decanediol, 1,2-decanediol, 1,2-undecanediol, 1,11-undecanediol, 1,12-dodecanediol, 1,2-dodecanediol, diethylene glycol, dipropylene glycol, triethylene glycol, tripropylene glycol, tetraethylene glycol, tetrapropylene glycol, pentaethylene glycol, pentapropylene glycol, hexaethylene glycol, hexapropylene glycol, heptaethylene glycol, heptapropylene glycol, octaethylene glycol, octapropylene glycol, nonaethylene glycol, nonapropylene glycol, decaethylene glycol, decapropylene glycol, cis- or trans-1,2-cylopentanediol, cis- or trans-1,3-cylopentanediol, cis- or trans-1,2-cylohexanediol, cis- or trans-1,3-cylohexanediol, cis- or trans-1,4-cylohexanediol, cis- or trans-1,2-cyloheptanediol, cis- or trans-1,3-cyloheptanediol, cis- or trans-1,4-cyloheptanediol, 1,2,3-cyclopentanetriol, 1,2,4-cyclopentanetriol, 1,2,3-cyclohexanetriol, 1,2,4-cyclohexanetriol, 1,2,3-cyloheptanetriol, 1,2,4-cyloheptanetriol, 1,2,3-propanetriol, 3-ethyl-2-hydroxymethyl-1,3-propanediol, 2-hydroxymethyl-2-methyl-1,3-propanediol, 2-hydroxymethyl-2-methyl-1,3-propanediol, 1,2,3-butanetriol, 1,2,4-butanetriol, 2-methyl-1,2,3-butanetriol, 2-methyl-1,2,4-butanetriol, 1,2,3-pentanetriol, 1,2,4-pentanetriol, 1,2,5-pentanetriol, 2,3,4-pentanetriol, 1,3,5-pentanetriol, 3-methyl-1,3,5-pentanetriol, 1,2,3-hexanetriol, 1,2,4-hexanetriol, 1,2,5-hexanetriol, 1,2,6-hexanetriol, 2,3,4-hexanetriol, 2,3,5-hexanetriol, 1,2,3-heptanetriol, 1,2,7-heptanetriol, 1,2,3-octanetriol, 1,2,8-octanetriol, 1,2,3-nonatriol, 1,2,9-nonatriol, 1,2,3-decanetriol, 1,2,10-decanetriol, 1,2,3-undecanetriol, 1,2,11-undecanetriol, 1,2,3-dodecanetriol, 1,1,12-dodecanetriol, 2,2,-bis(hydroxymethyl)-1,3-propanediol, 1,2,3,4-butanetetraol, 1,2,3,4-pentanetetraol, 1,2,3,5-pentanetetraol, 1,2,3,4-hexanetetraol, 1,2,3,6-hexanetetraol, 1,2,3,4-heptanetetraol, 1,2,3,7-heptanetetraol, 1,2,3,4-octanetetraol, 1,2,3,8-octanetetraol, 1,2,3,4-nonanetetraol, 1,2,3,9-nonanetetraol, 1,2,3,4-decanetetraol, 1,2,3,10-decanetetraol, trimethylolpropanol, pentaerythritol, sugar alcohols such as mannitol, sorbitol or arabitol, hexanehexol, 1,2,3,4,5-pentanepentol and 1,2,3,4,5,6-hexanehexaol.

In some embodiments, the polyol is 1,2,3-propanetriol (also referred to as glycerol).

In some embodiments, a polyol may be present in an amount ranging from between about 1% to about 10% by total volume of the composition; from between about 2% to about 8% by total volume of the composition; from between about 3% to about 7% by total volume of the composition; from between about 4% to about 6% by total volume of the composition.

### Examples of Compositions

One example of a composition comprises a set of primers; a polymerase; and betaine. In some embodiments, the betaine is present at a concentration ranging from between about 0.3 mM to about 0.7 mM. In some embodiments, the concentration of betaine is about 0.5 mM. In some embodiments, at least one primer of the set of primers has a high melting temperature and/or a high GC content (e.g., a melting temperature greater than 63° C.) In some embodiments, a plurality of the primers of the set of primers each have a high melting temperature and/or a high GC content (e.g., a melting temperature greater than 63° C.). In some embodiments, there are no restrictions or limits placed on the melting temperatures and/or the GC contents of any of the primers within the set of primers. In some embodiments, up to about 10% of the primers in the composition have high melting temperatures and/or high GC content. In some embodiments, the composition further comprises a mixture of modified and modified dNTPs. In some embodiments, a ratio of modified dNTPs to unmodified dNTPs is about 1:1. In some embodiments, the composition further comprises at least one of a buffer, a divalent cation, and a polyol. In some embodiments, the composition comprises at least two of a buffer, a divalent cation, and a polyol. In some embodiments, the composition comprises each of a buffer, a divalent cation, and a polyol. In some embodiments, the composition is useful in the amplification of nucleic acid molecules, such as nucleic acid molecules present in a nucleic acid library. In some embodiments, the composition is useful in pre-capture amplification. In some embodiments, the composition is useful in one or more steps of target enrichment, such as during the hybridization and extension of a capture primer (as described further herein).

Another example of a composition comprises a set of primers; a polymerase; and optionally betaine; where at least one primer of the set of primers has a high melting temperature and/or a high GC content. In some embodiments, the at least one primer of the set of primers has a melting temperature greater than about 64° C., greater than about 65° C., greater than about 66° C., greater than about 67° C., greater than about 68° C., greater than about 69° C., greater than about 70° C., greater than about 71° C., greater than about 72° C., greater than about 73° C., greater than about 74° C., greater than about 75° C., greater than about 76° C., greater than about 77° C., greater than about 78° C., greater than about 79° C., greater than about 80° C., greater than about 81° C., greater than about 82° C., greater than about 83° C., greater than about 84° C., greater than about 85° C., greater than about 86° C., greater than about 87° C., greater than about 88° C., greater than about 89° C., greater than about 90° C., greater than about 91° C., least about 92° C., greater than about 93° C., greater than about 94° C., greater than about 95° C., greater than about 96° C., greater than about 97° C., greater than about 98° C., greater than about 99° C., greater than about 100° C., etc. In some embodiments, a plurality of the primers of the set of primers each have a high melting temperature and/or a high GC content (e.g., a melting temperature greater than 63° C.). In some embodiments, there are no restrictions or limits placed on the melting temperatures and/or the GC contents of any of the primers within the set of primers. In some embodiments, up to about 10% of the primers in the composition have high melting temperatures and/or high GC content. In some embodiments, the betaine is present at a concentration ranging from between about 0.3 mM to about 0.7 mM. In some embodiments, the composition further comprises a mixture of modified and modified dNTPs. In some embodiments, a ratio of modified dNTPs to unmodified dNTPs is about 1:1. In some embodiments, the composition further comprises at least one of a buffer, a divalent cation, and a polyol. In some embodiments, the composition comprises at least two of a buffer, a divalent cation, and a polyol. In some embodiments, the composition comprises each of a buffer, a divalent cation, and a polyol. In some embodiments, the composition is useful in the amplification of nucleic acid molecules, such as nucleic acid molecules present in a nucleic acid library. In some embodiments, the composition is useful in pre-capture amplification. In some embodiments, the composition is useful in one or more steps of target enrichment, such as during the hybridization and extension of a capture primer (as described further herein).

### Methods

The present disclosure is also directed to methods of enriching a plurality of target nucleic acid molecules in a sample, e.g., between about one and about ten thousand target nucleic acid molecules, or between about one and about five thousand target nucleic acid molecules, or between about one and about 1000 target nucleic acid molecules.

The present disclosure is also directed to methods of amplifying a target enriched sample, such as a target enriched sample prepared using any one of the methods described herein. Additionally, the present disclosure is also directed to methods of sequencing using a target enriched sample, such as a target enriched sample prepared using any one of the methods described herein. The methods of the present disclosure can be used as a part of a sequencing protocol, including a high throughput single molecule sequencing protocol. In some embodiments, the method of the present disclosure generates a library of enriched target nucleic acid molecules to be sequenced. The enriched target nucleic acid molecules in the library may optionally incorporate barcodes for molecular identification and sample identification, such as described in U.S. Publication No. 2020/0032244, and in U.S. Pat. Nos. 7,393,665, 8,168,385, 8,481,292, 8,685,678, and 8,722,368, the disclosures of which are hereby incorporated by reference herein in their entireties.

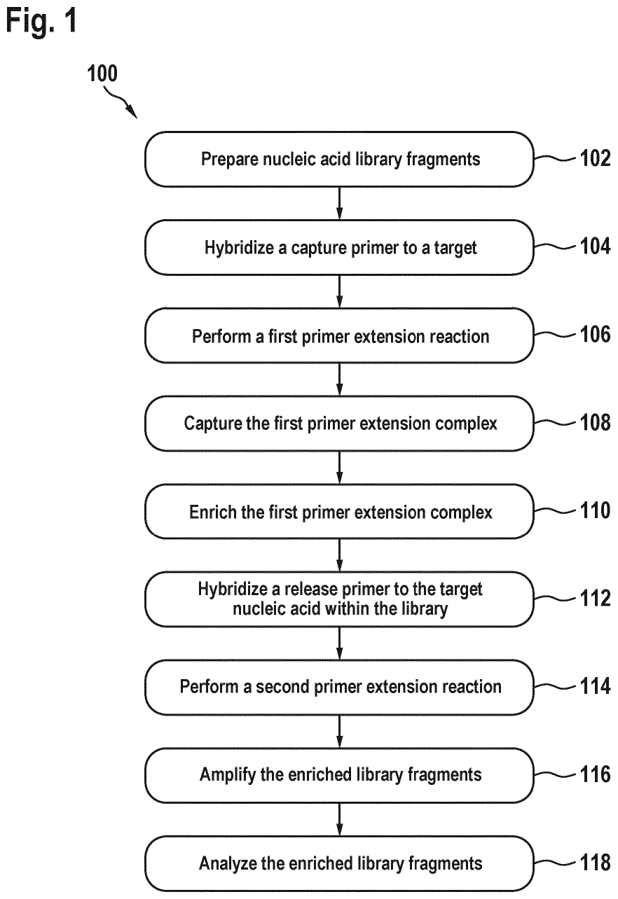

Referring to FIG. 1, a method 100 for target enrichment by unidirectional dual probe primer extension includes a step 102 of preparing a nucleic acid library for enrichment. In some embodiments, step 102 includes fragmentation (optional), adapter ligation (to each end of the nucleic acid molecules), and pre-capture amplification. Ultimately, the product of the step 102 results in a library of nucleic acid molecules, where each of the nucleic acid molecules in the library of nucleic acid molecules has a first end comprising a first adapter and a second end comprising a second adapter. In some embodiments, the first and second adapter may be the same or different.

In some embodiments, the nucleic acid library can be prepared from any source of nucleic acid molecules including one or more target nucleic acid molecules. In general, a target nucleic acid will include a region or sequence of interest, and the method 100 facilitates the preferential enrichment of the one or more target nucleic acid molecules relative to non-target nucleic acid molecules in the nucleic acid library for downstream detection and analysis of those regions or sequences of interest.

Following ligation of the adapters, the adapter ligated (and optionally fragmented) nucleic acid molecules are amplified (pre-capture amplification). In some embodiments, the pre-capture amplification takes place using any of the compositions described herein. For example, a suitable composition (e.g., a pre-capture amplification master mix composition) may include one or more primers; one or more polymerases; dNTPs (e.g., unmodified dNTPs and mixtures of unmodified and modified dNTPs); and at least one enhancer. By way of another example, a pre-capture master mix composition may include one or more primers, where at least one primer of the one or more primers has a high melting temperature (or a high GC content) and/or includes one or more modified dNTPs; one or more polymerases; dNTPs; and optionally at least one enhancer (e.g., betaine, dimethyl sulfoxide (DMSO), a trehalose, and/or a single stranded DNA binding protein, such as in the amounts/concentrations described herein).

In some embodiments, pre-capture amplification may be performed with a set of pre-capture primers (e.g., forward and reverse pre-capture primers) where at least one primer in the set of pre-capture primers has a high GC content and/or a high melting temperature (e.g., a melting temperature greater than 63° C., greater than about 66° C., greater than about 69° C., greater than about 72° C., greater than about 75° C., etc. In some embodiments, the pre-capture primers introduced may have no restriction on GC content and/or melting temperature. In some embodiments, up to about 25% of the primers utilized during pre-capture amplification are high melting temperature and/or high GC content primers. In some embodiments, up to about 20% of the primers utilized during pre-capture amplification are high melting temperature and/or high GC content primers. In some embodiments, up to about 15% of the primers utilized during pre-capture amplification are high melting temperature and/or high GC content primers. In some embodiments, up to about 12% of the primers utilized during pre-capture amplification are high melting temperature and/or high GC content primers. In some embodiments, up to about 10% of the primers utilized during pre-capture amplification are high melting temperature and/or high GC content primers. In some embodiments, up to about 9% of the primers utilized during pre-capture amplification are high melting temperature and/or high GC content primers. In some embodiments, up to about 8% of the primers utilized during pre-capture amplification are high melting temperature and/or high GC content primers. In some embodiments, up to about 7% of the primers utilized during pre-capture amplification are high melting temperature and/or high GC content primers. In some embodiments, up to about 6% of the primers utilized during pre-capture amplification are high melting temperature and/or high GC content primers. In some embodiments, up to about 5% of the primers utilized during pre-capture amplification are high melting temperature and/or high GC content primers. In some embodiments, up to about 4% of the primers utilized during pre-capture amplification are high melting temperature and/or high GC content primers. In some embodiments, up to about 3% of the primers utilized during pre-capture amplification are high melting temperature and/or high GC content primers. In some embodiments, up to about 2% of the primers utilized during pre-capture amplification are high melting temperature and/or high GC content primers. In some embodiments, up to about 1% of the primers utilized during pre-capture amplification are high melting temperature and/or high GC content primers.

In other embodiments, pre-capture amplification may be performed in the presence of a set of pre-capture primers (e.g., forward and reverse pre-capture primers) where at least one primer of the set of primers has (i) a high GC content and/or a high melting temperature; and (ii) which include one or more modified dNTPs (7-deaza dGTP and/or 2-amino dATP). For example, the primers introduced may have melting temperatures greater than 63° C., greater than about 66° C., greater than about 69° C., greater than about 72° C., greater than about 75° C., etc.; and where the primers include one or more modified dNTPs. In some embodiments, the primers introduced may have no restriction on GC content and/or melting temperature; and where the primers also include one or more modified dNTPs.

In yet other embodiments, pre-capture amplification takes place in the presence of one or more enhancers, e.g., betaine, DMSO, single stranded DNA binding proteins, disaccharides, or any combination thereof. For instance, pre-capture amplification may be performed utilizing a composition which includes betaine (or a derivative or analog thereof), such as a composition including betaine at a concentration ranging from between about 0.1 mM to about 1 mM (e.g., a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, etc.).

In further embodiments, the pre-capture amplification takes place in a composition which includes one or more pre-capture primers (e.g., forward and reverse pre-capture primers) that have a high GC content and/or a high melting temperature; and where the composition further includes at least one enhancer. In some embodiments, the enhancer is betaine (or a derivative or analog thereof) and a concentration of betaine in the composition ranges from between about 0.2 mM to about 0.8 mM. In other embodiments, the enhancer is betaine (or a derivative or analog thereof) and a concentration of betaine in the composition ranges from between about 0.3 mM to about 0.7 mM. In yet other embodiments, the enhancer is betaine (or a derivative or analog thereof) and a concentration of betaine in the composition ranges from between about 0.4 mM to about 0.6 mM. In further embodiments, the enhancer is betaine (or a derivative or analog thereof) and a concentration of betaine in the composition is about 0.5 mM.

In even further embodiments, pre-capture amplification may be performed in a composition which includes a mixture of unmodified dNTPs and modified dNTPs. In some embodiments, a ratio of unmodified dNTPs to modified dNTPs in any composition ranges from between about 2:1 to about 1:2. In some embodiments, the ratio of unmodified dNTPs to modified dNTPs is about 1:1. In some embodiments, pre-capture amplification may take place in a composition which include one or more modified dNTPs (such as at a concentration ranging from between about 0.2 mM to about 0.8 mM, at a concentration ranging from between about 0.3 mM to about 0.7 mM, at a concentration ranging from between about 0.3 mM to about 0.6 mM, etc.). In some embodiments, the concentration of each different modified dNTP in the composition is about 0.3 mM. n some embodiments, the one or more modified dNTPs include modified dGTPs and/or modified dATPs. In some embodiments, the one or more modified dNTPs are 7-Deaza-2′-deoxyguanosine-5′-Triphosphates and/or 2-Amino-2′ deoxyadenosine-5′-Triphosphates. In some embodiments, a concentration of 7-Deaza-2′-deoxyguanosine-5′-Triphosphates and/or 2-Amino-2′ deoxyadenosine-5′-Triphosphates is about the same as the concentration of unmodified dNTPs in the composition.

In some embodiments, pre-capture amplification may take place in the presence of a polymerase, betaine, and a mixture of unmodified and modified dNTPs. In these embodiments, the betaine is present at a concentration ranging from about 0.4 mM to about 0.6 mM; and where a ratio of the unmodified dNTPs to modified dNTPs is about 1:1. In some embodiments, the modified dNTPs are selected from 7-Deaza-2′-deoxyguanosine-5′-Triphosphates or 2-Amino-2′ deoxyadenosine-5′-Triphosphates.

In some embodiments, pre-capture amplification may take place in the presence of a polymerase, betaine, an optional mixture of unmodified and modified dNTPs, one or more buffers, and one or more divalent cations. In these embodiments, the betaine is present at a concentration ranging from about 0.4 mM to about 0.6 mM; and where a ratio of the unmodified dNTPs to modified dNTPs is about 1:1. In some embodiments, the betaine is present at a concentration ranging from about 0.4 mM to about 0.6 mM; a ratio of the unmodified dNTPs to modified dNTPs is about 1:1; and the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates.

In some embodiments, pre-capture amplification may take place in the presence of a polymerase, a mixture of unmodified and modified dNTPs, one or more buffers, and one or more divalent cations. In these embodiments, a ratio of the unmodified dNTPs to modified dNTPs is about 1:1. In some embodiments, a ratio of the unmodified dNTPs to modified dNTPs is about 1:1; and where the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates.

Examples of pre-capture amplification master mixed are set forth in Table 1 below:

TABLE 1
Pre-capture amplification master mixes which may be
utilized when performing step 102 of method 100.

| | | | Final Reaction Amount | | | | |
|---|---|---|---|---|---|---|---|
| | | | | | Betaine | | |
| | | | | | and 7- | 7- | |
| | | | | | Deaza | Deaza | |
| | Reagent | Stock | | Betaine | dGTP | dGTP | |
| Component | Name | Amount | Control | Added | Added | Added | Units |
| Enzyme and | HiFi HotStart | 2 | 1 | 1 | 1 | 1 | X |
| Buffer Mix | Ready Mix | | | | | | |
| | (available from | | | | | | |
| | Roche | | | | | | |
| | Sequencing | | | | | | |
| | Solutions, Inc.) | | | | | | |
| Forward & | KAPA UDI | 10 | 1 | 1 | 1 | 1 | X |
| Reverse | Primer Mixes | | | | | | |
| Primer | (available from | | | | | | |
| | Roche | | | | | | |
| | Sequencing | | | | | | |
| | Solutions, Inc.) | | | | | | |
| Betaine | | | | 0.5 | 0.5 | | mM |
| 7-deaza | | | | | 0.3 | 0.3 | mM |
| dGTP | | | | | | | |

Following pre-capture amplification at step 102, step 104 of method 100 includes hybridization of a capture primer to a target nucleic acid present in the library of nucleic acid molecules, thereby forming an unextended capture primer-target complex. In some embodiments, the capture primer is a target-specific primer having a defined sequence that is complementary to a sequence of a target nucleic acid. One example of a target-specific primer is a gene-specific primer designed to hybridize to or nearby (e.g., upstream of, or 5′ to) a gene (e.g., cDNA, genomic DNA) of interest. The target nucleic acid can be RNA, DNA, or a combination thereof. The capture primer can be an oligonucleotide primer composed of ribonucleic acids, deoxyribonucleic acids, modified nucleic acids (e.g., biotinylated, locked nucleic acids, inosines, Seela bases, or the like), or other nucleic acid analogs known in the art.

In some embodiments, the capture primer can include one or more modified bases, capture moieties or a combination thereof. In the case that the capture primer includes a capture moiety, the capture primer can be attached to a solid support or be free in solution (i.e., not bound or otherwise attached to a solid support) prior to the step 104 of hybridizing the capture primer to the target nucleic acid. Other suitable capture moieties and their utility are described in United States Publication No. 2020/0032244, the disclosure of which is hereby incorporated by reference herein in its entirety). In embodiments where the capture primer including a capture moiety is not attached to a solid support via the capture moiety, the step 104 can be conducted in solution. In embodiments where the capture primer including a capture moiety is attached to a solid support via the capture moiety, the step 104 can be conducted situ. Notably, in the case of an in-situ reaction, the resulting unextended primer-target complex will be attached to a solid support. Any non-target nucleic acid molecules or target nucleic acid molecules not annealed to the capture primer that remain in solution can be removed by separating the solution from the solid support to which primer-target complexes are bound.

At step 106, a capture primer extension reaction is performed. In some embodiments, step 106 includes extension of the hybridized capture primer with a first polymerase, thereby generating a capture primer extension product or complex including a 3′ region of the extended capture primer comprising the reverse complement of at least a portion of the target nucleic acid template. In some embodiments, the hybridization and extension reactions (steps 104 and 106) are optionally performed simultaneously, whereas in other embodiments, the hybridization and extension reactions are performed separately (e.g., sequentially) and may be separated by a wash step removing the non-annealed and not captured target nucleic acid molecules from the reaction mixture.

In embodiments where the capture primer is hybridized and extended in the same step, referred to as “capture extension,” the capture extension may take place using any of the compositions described herein, e.g., a composition including one or more capture primers; one or more polymerases; dNTPs (including unmodified dNTPs or a mixture of unmodified and modified dNTPs); and at least one enhancer (e.g., betaine). In other embodiments, the capture extension takes place in a composition including one or more capture primers, where at least one capture primer of the one or more capture primers has a high melting temperature and/or includes one or more modified dNTPs; one or more polymerases; dNTPs (including unmodified dNTPs or a mixture of unmodified and modified dNTPs); and optionally at least one enhancer (e.g., betaine).

In some embodiments, capture extension may be performed in the presence of a set of capture primers, where at least one primer in the set of capture primers has a high GC content and/or a high melting temperature (e.g., a melting temperature greater than 63° C., greater than about 66° C., greater than about 69° C., greater than about 72° C., greater than about 75° C., etc.). In some embodiments, the capture primers introduced may have no restriction on GC content and/or melting temperature.

In some embodiments, capture extension may be performed in the presence of a set of pre-capture primers (e.g., forward and reverse pre-capture primers) where at least one primer in the set of pre-capture primers has a high GC content and/or a high melting temperature (e.g., a melting temperature greater than 63° C., greater than about 66° C., greater than about 69° C., greater than about 72° C., greater than about 75° C., etc. In some embodiments, the pre-capture primers introduced may have no restriction on GC content and/or melting temperature.

In some embodiments, up to about 25% of the capture primers of any set of capture primers are high melting temperature and/or high GC content capture primers. In some embodiments, up to about 20% of the capture primers of any set of capture primers are high melting temperature and/or high GC content capture primers. In some embodiments, up to about 15% of the capture primers of any set of capture primers are high melting temperature and/or high GC content capture primers. In some embodiments, up to about 12% of the capture primers of any set of capture primers are high melting temperature and/or high GC content capture primers. In some embodiments, up to about 10% of the capture primers of any set of capture primers are high melting temperature and/or high GC content capture primers. In some embodiments, up to about 9% of the capture primers of any set of capture primers are high melting temperature and/or high GC content capture primers. In some embodiments, up to about 8% of the capture primers of any set of capture primers are high melting temperature and/or high GC content capture primers. In some embodiments, up to about 7% of the capture primers of any set of capture primers are high melting temperature and/or high GC content capture primers. In some embodiments, up to about 6% of the capture primers of any set of capture primers are high melting temperature and/or high GC content capture primers. In some embodiments, up to about 5% of the capture primers priers of any set of capture primers are high melting temperature and/or high GC content capture primers. In some embodiments, up to about 4% of the capture primers of any set of capture primers are high melting temperature and/or high GC content capture primers. In some embodiments, up to about 3% of the capture primers of any set of capture primers are high melting temperature and/or high GC content capture primers. In some embodiments, up to about 2% of the capture primers of any set of capture primers are high melting temperature and/or high GC content capture primers. In some embodiments, up to about 1% of the capture primers of any set of capture primers are high melting temperature and/or high GC content capture primers.

In other embodiments, capture extension may be performed in the presence of a set of capture primers where at least one primer of the set of primers has (i) a high GC content and/or a high melting temperature; and (ii) which include one or more modified dNTPs. For example, the primers introduced may have melting temperatures greater than 63° C., greater than about 66° C., greater than about 69° C., greater than about 72° C., greater than about 75° C., etc.; and where the primers include one or more modified dNTPs. In some embodiments, the primers introduced may have no restriction on GC content and/or melting temperature; and where the primers also include one or more modified dNTPs.

In yet other embodiments, capture extension may take place in the presence of one or more enhancers, e.g., betaine, DMSO, single stranded DN binding proteins, disaccharides, or any combination thereof. For instance, capture extension may be performed utilizing a composition which includes betaine (or a derivative or analog thereof), such as a composition including betaine at a concentration ranging from between about 0.1 mM to about 1 mM (e.g., a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, etc.).

In further embodiments, the capture extension may be performed in a composition which includes one or more capture primers that have a high GC content and/or a high melting temperature; and where the composition further includes at least one enhancer. In some embodiments, the enhancer is betaine (or a derivative or analog thereof) and a concentration of betaine in the composition ranges from between about 0.2 mM to about 0.8 mM. In other embodiments, the enhancer is betaine (or a derivative or analog thereof) and a concentration of betaine in the composition ranges from between about 0.3 mM to about 0.7 mM. In yet other embodiments, the enhancer is betaine (or a derivative or analog thereof) and a concentration of betaine in the composition ranges from between about 0.4 mM to about 0.6 mM. In further embodiments, the enhancer is betaine (or a derivative or analog thereof) and a concentration of betaine in the composition is about 0.5 mM.

In even further embodiments, capture extension may be performed in a composition which includes a mixture of unmodified dNTPs and modified dNTPs. In some embodiments, a ratio of unmodified dNTPs to modified dNTPs in any composition ranges from between about 2:1 to about 1:2. In some embodiments, the ratio of unmodified dNTPs to modified dNTPs is about 1:1. In some embodiments, capture extension may take place in a composition which includes one or more modified dNTPs (such as at a concentration ranging from between about 0.2 mM to about 0.8 mM, at a concentration ranging from between about 0.3 mM to about 0.7 mM, at a concentration ranging from between about 0.3 mM to about 0.6 mM, etc.). In some embodiments, the one or more modified dNTPs include modified dGTPs and/or modified dATPs. In some embodiments, the one or more modified dNTPs are 7-Deaza-2′-deoxyguanosine-5′-Triphosphates and/or 2-Amino-2′ deoxyadenosine-5′-Triphosphates. In some embodiments, a concentration of 7-Deaza-2′-deoxyguanosine-5′-Triphosphates and/or 2-Amino-2′ deoxyadenosine-5′-Triphosphates is about the same as the concentration of unmodified dNTPs in the composition.

In some embodiments, capture extension may take place in the presence of a polymerase, betaine, and a mixture of unmodified and modified dNTPs. In these embodiments, the betaine is present at a concentration ranging from about 0.4 mM to about 0.6 mM; and where a ratio of the unmodified dNTPs to modified dNTPs is about 1:1. In some embodiments, the modified dNTPs are selected from 7-Deaza-2′-deoxyguanosine-5′-Triphosphates or 2-Amino-2′ deoxyadenosine-5′-Triphosphates.

In some embodiments, capture extension may take place in the presence of a polymerase, betaine, an optional mixture of unmodified and modified dNTPs, one or more buffers, and one or more divalent cations. In these embodiments, the betaine is present at a concentration ranging from about 0.4 mM to about 0.6 mM; and where a ratio of the unmodified dNTPs to modified dNTPs is about 1:1. In some embodiments, the betaine is present at a concentration ranging from about 0.4 mM to about 0.6 mM; a ratio of the unmodified dNTPs to modified dNTPs is about 1:1; and the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates. In some embodiments, the divalent cations are present at a concentration of about 0.1 mM to about 0.4 mM.

In some embodiments, capture extension may take place in the presence of a polymerase, a mixture of unmodified and modified dNTPs, one or more buffers, and one or more divalent cations. In these embodiments, a ratio of the unmodified dNTPs to modified dNTPs is about 1:1. In some embodiments, a ratio of the unmodified dNTPs to modified dNTPs is about 1:1; and the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates.

Examples of capture extension master mixes that may be utilized when performing step 106 are set forth in Table 2 below.

TABLE 2
Capture extension master mixes which may be utilized when
performing the capture extension steps of method 100.

| | | | Final Reaction | | |
|---|---|---|---|---|---|
| | | | Amount | | |
| | | | | Betaine | |
| Component | Reagent Name | Stock | Control | Added | Units |
| Buffer and | Capture Extension | 5 | 1 | 1 | X |
| Enzyme | Reagent | | | | |
| Mix | (available from Roche | | | | |
| | Sequencing Solutions, | | | | |
| | Inc.) | | | | |
| Fragmented DNA | Input library | 875 - 7500 | 500 - 3000 | 500 - 3000 | ng |
| and Buffer | | | | | |
| Capture primers | Capture Panel | 10 | 1 | 1 | X |
| and Buffers | | | | | |
| Betaine | | | | 0.5 | mM |

In addition to providing compositions for conducting step 106 of method 100, the present disclosure also provides for methods of incubating a sample. In particular, the present disclosure provides incubation programs (including denaturation, annealing and extension portions) having varying temperatures and times which may be utilized to improve uniformity of coverage and/or to reduce GC bias during one or more downstream sequencing operations. Of course, any of the incubation programs described herein may be utilized in conjunction with any of the compositions disclosed herein.

In some embodiments, the denaturing, annealing and extension portions of incubation may be conducted over a cumulative time period ranging from about 20 minutes to about 40 minutes, e.g., between about 25 minutes and about 35 minutes. By way of example, a cumulative time period for conducting the denaturing, annealing and extension portions of step 106 of method 100 may be about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, etc.

In some embodiments, a cumulative time period for conducting the annealing and extension portions of incubation ranges from between about 20 minutes to about 25 minutes. In some embodiments, a cumulative time period for conducting the annealing and extension portions of incubation is about 23 minutes. In other embodiments, a cumulative time period for conducting the annealing and extension portions of incubation from between about 30 minutes to about 34 minutes. In other embodiments, a cumulative time period for conducting the annealing and extension portions of incubation is about 32 minutes.

In some embodiments, annealing and extension is conducted at temperatures ranging from between about 60° C. to about 80° C., such as illustrated in Tables 3-7. In some embodiments, the incubation programs of the present disclosure include one or more pauses between annealing temperatures as compared with control incubation programs. For instance, Program 1 adds pauses at regular intervals between 80° C. and 60° C. while keeping the overall program length the same as a control program (Program 5). Similarly, Program 2 adds pauses at higher temperature intervals between 80° C. and 60° C. while keeping the overall program length the same as the control program (Program 5). Program 3 adds pauses at higher temperature intervals between 80° C. and 60° C. while matching a total incubation time to the control program (Program 5) incubation at 60° C., 10 min, thereby increasing the total program time by 10 minutes. Program 4 adds pauses at regular intervals between 80° C. and 60° C. while matching incubation time to the control program (Program 5) incubation at 60° C., 10 min, thereby increasing the total program time by 10 minutes

TABLE 3

| Exemplary incubation program having a | | |
|---|---|---|
| total time of about 25 minutes. | | |
| | Program 1 | |
| Step | Temp. | Time |
| Denaturation | 95 °C | 2 min. |
| Annealing and | 80 °C | 3 min. |
| Extension | 75 °C | 4.5 min. |
| | 70 °C | 4.5 min. |
| | 65 °C | 4.5 min. |
| | 60 °C | 4.5 min. |
| | 65 °C | 2 min. |
| | Total: | about 25 min. |

TABLE 4

| Exemplary incubation program having a | | |
|---|---|---|
| total time of about 25 minutes. | | |
| | Program 2 | |
| Step | Temp. | Time |
| Denaturation | 95 °C | 2 min. |
| Annealing and | 80 °C | 1 min. |
| Extension | 75 °C | 5 min. |
| | 70 °C | 5 min. |
| | 60 °C | 10 min. |
| | 65 °C | 2 min. |
| | Total: | about 25 min. |

TABLE 5

| Exemplary incubation program having a | | |
|---|---|---|
| total time of about 34 minutes. | | |
| | Program 3 | |
| Step | Temp. | Time |
| Denaturation | 95 °C | 2 min. |
| Annealing and | 80 °C | 1 sec. |
| Extension | Slow ramp | Not measured |
| | 75 °C | 10 min. |
| | 70 °C | 10 min. |
| | 60 °C | 10 min. |
| | 65 °C | 2 min. |
| | Total: | about 34 min. |

TABLE 6

| Exemplary incubation program having a | | |
|---|---|---|
| total time of about 34 minutes. | | |
| | Program 4 | |
| Step | Temp. | Time |
| Denaturation | 95 °C | 2 min. |
| Annealing and | 80 °C | 1 sec. |
| Extension | Slow ramp | Not measured |
| | 72 °C | 10 min. |
| | 66 °C | 10 min. |
| | 60 °C | 10 min. |
| | 65 °C | 2 min. |
| | Total: | about 34 min. |

TABLE 7

| Exemplary incubation program having a | | |
|---|---|---|
| total time of about 24 minutes. | | |
| | Program 5 (Control) | |
| Step | Temp. | Time |
| Denaturation | 95 °C | 2 min. |
| Annealing and | 80 °C | 1 sec. |
| Extension | | |
| | Slow ramp | 10 min. |
| | 60 °C | 10 min. |
| | 65 °C | 2 min. |
| | Total: | about 24 min. |

The method 100 further includes a step 108 of capturing the capture primer extension complex. Capture of the capture primer extension complex can be achieved in a variety of ways as disclosed herein and can be achieved prior to, concurrent with, or subsequent to either of the step 104 and the step 106 of the method 100. As described above, the capture oligonucleotide can include a capture moiety that can be used to capture the capture primer onto a solid support before, during, or after the step 104 or the step 106 of the method 100. In another example, extension of the capture primer following hybridization to the target nucleic acid includes incorporation of one or more modified nucleotides. The modified nucleotides can include a capture moiety or may be configured to enable downstream modification of the modified nucleotides to attach or otherwise incorporate a capture moiety into the extended portion of the capture primer extension complex. Accordingly, the capture primer extension complex can be captured during or subsequent to the step 106 by way of the capture moieties associated with the one or more modified nucleotides. The choice of whether the target nucleic acid, the annealed primer-target complex, or the target-extended primer complex is captured further determines whether the step 104 and the step 106 of the method are performed in solution or in situ.

Step 110 of method 110 comprises enrichment of the capture primer extension complex. In some embodiments, step 110 includes one or more purification and enrichment steps for recovery of the capture primer extension complex from non-target nucleic acid molecules in the library and other molecules such as unused reaction components (e.g., nucleotides, primer molecules, ATP, etc.), enzymes, buffers, or the like. In some embodiments, step 110 includes enzymatic digestion, size-exclusion based purification, affinity-based purification, the like, or a combination thereof. In some embodiments, enrichment involves increasing the concentration of the target nucleic acid through depletion (i.e., removal) of other members of the library of nucleic acid molecules that are not target nucleic acid molecules.

Hybridization of a release primer to a target nucleic acid takes place at step 112 of method 100. In some embodiments, the release primer is a target specific primer that binds to a region of interest within the target nucleic acid (as opposed to hybridizing with or being complementary to one or both of the first adapter and the second adapter introduced at step 102). In some embodiments, the target nucleic is a part of the capture primer extension complex synthesized at step 112. For example, the release primer can hybridize to the target nucleic acid at a 5′ position (i.e., upstream) relative to the extended capture primer in the capture primer extension complex. The resulting unextended release primer-target complex in this case includes the extended capture primer, the target nucleic acid hybridized to the extended capture primer, and the second (unextended) oligonucleotide primer. In the case that the capture primer extension product is attached to a solid support during the step 112, the unextended release primer-target complex will similarly be attached to the solid support. In other embodiments, (e.g., after removal of the non-target nucleic acid molecules from the reaction mixture) the capture primer extension product is freed from the solid support and is in solution to enable in solution hybridization of the release primer in the step 112.

Step 114 of method 100 includes performing a release primer extension reaction. Following hybridization of the release primer to the target nucleic acid template in the step 112, the release primer is extended by a second polymerase, thereby generating a release primer extension product or complex including the target nucleic acid. The extended-release primer includes a 3′ region comprising the reverse complement of at least a portion of the target nucleic acid template. In some embodiments, extension of the release primer with the second polymerase liberates the extended capture primer from the complex with the target nucleic acid. Liberating the extended capture oligonucleotide from the capture primer extension complex can include one or more of strand displacement (e.g., by a polymerase), or digestion (e.g., by a nuclease). For example, liberating the extended capture oligonucleotide can be achieved with an enzyme having at least one of a strand-displacing activity, a 5′ to 3′ exonuclease activity, and a flap endonuclease activity. In some embodiments, the first and second polymerases are the same. In other embodiments, the first and second polymerases are different.

Steps 112 and 114 may be performed sequentially or simultaneously. Regardless of whether steps 112 and 114 are performed sequentially or simultaneously, any of the release primers utilized may have high melting temperatures and/or high GC contents. In some embodiments, at least one release primer of a set of release primers has a melting temperature greater than 63° C., greater than about 66° C., greater than about 69° C., greater than about 72° C., greater than about 75° C., greater than about 80° C., etc. In some embodiments, a plurality of release primers of a set of release primers has a melting temperature greater than 63° C., greater than about 66° C., greater than about 69° C., greater than about 72° C., greater than about 75° C., greater than about 80° C., etc. In some embodiments, there are no restrictions on the melting temperatures and/or the GC content of the release primers. In some embodiments, the release primers may include one or more modified dNTPs, such as two or more modified dNTPs, three or more modified dNTPs, four or more modified dNTPs, etc.

In some embodiments, up to about 25% of the release primers in any set of release primers are high melting temperature and/or high GC content release primers. In some embodiments, up to about 20% of the release primers of any set of release primers are high melting temperature and/or high GC content release primers. In some embodiments, up to about 15% of the release primers of any set of release primers are high melting temperature and/or high GC content release primers. In some embodiments, up to about 12% of the release primers of any set of release primers are high melting temperature and/or high GC content release primers. In some embodiments, up to about 10% of the release primers of any set of release primers are high melting temperature and/or high GC content release primers. In some embodiments, up to about 9% of the release primers of any set of release primers are high melting temperature and/or high GC content release primers. In some embodiments, up to about 8% of the release primers of any set of release primers are high melting temperature and/or high GC content release primers. In some embodiments, up to about 7% of the release primers of any set of release primers are high melting temperature and/or high GC content release primers. In some embodiments, up to about 6% of the release primers of any set of release primers are high melting temperature and/or high GC content release primers. In some embodiments, up to about 5% of the release primers of any set of release primers are high melting temperature and/or high GC content release primers. In some embodiments, up to about 4% of the release primers of any set of release primers are high melting temperature and/or high GC content release primers. In some embodiments, up to about 3% of the release primers of any set of release primers are high melting temperature and/or high GC content release primers. In some embodiments, up to about 2% of the release primers of any set of release primers are high melting temperature and/or high GC content release primers. In some embodiments, up to about 1% of the release primers of any set of release primers are high melting temperature and/or high GC content release primers.

In embodiments where steps 112 and 114 are performed sequentially, step 112 may be conducted in the presence of at least one enhancer selected from betaine (or a derivative or analog thereof), DMSO, a disaccharide, and a single stranded DNA binding protein (SSB). In some embodiments, the at least one enhancer is betaine, and a concentration of betaine in any release primer hybridization master mix used during the performance of step 112 includes a concentration of betaine of about 0.05 mM, about 0.1 mM, about 0.2 mM, about 0.25 mM, about 0.3 mM, about 0.35 mM, about 0.4 mM, about 0.5 mM, about 0.55 mM, about 0.6 mM, about 0.65 mM, about 0.7 mM, about 0.8 mM, about 0.9 mM, about 1 mM, etc.

In embodiments where steps 112 and 14 are conducted simultaneously, the combined release primer hybridization and extension may take place in the presence of one or more enhancers, e.g., betaine, DMSO, single stranded DN binding proteins, disaccharides, or any combination thereof. For instance, combined release primer hybridization and extension may be performed utilizing a composition which includes betaine (or a derivative or analog thereof), such as a composition including betaine at a concentration ranging from between about 0.1 mM to about 1 mM (e.g., a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, etc.).

In some embodiments, combined release primer hybridization and extension may be performed in a composition which includes a mixture of unmodified dNTPs and modified dNTPs. In some embodiments, a ratio of unmodified dNTPs to modified dNTPs in any composition ranges from between about 2:1 to about 1:2. In some embodiments, the ratio of unmodified dNTPs to modified dNTPs is about 1:1. In some embodiments, the one or more modified dNTPs include modified dGTPs and/or modified dATPs. In some embodiments, the one or more modified dNTPs are 7-Deaza-2′-deoxyguanosine-5′-Triphosphates and/or 2-Amino-2′ deoxyadenosine-5′-Triphosphates. In some embodiments, a concentration of 7-Deaza-2′-deoxyguanosine-5′-Triphosphates and/or 2-Amino-2′ deoxyadenosine-5′-Triphosphates is about the same as the concentration of unmodified dNTPs in the composition.

In other embodiments, combined release primer hybridization and extension takes place in a composition which includes one or more capture extension that have a high GC content and/or a high melting temperature; and where the composition further includes at least one enhancer. In some embodiments, the enhancer is betaine (or a derivative or analog thereof) and a concentration of betaine in the composition ranges from between about 0.2 mM to about 0.8 mM. In other embodiments, the enhancer is betaine (or a derivative or analog thereof) and a concentration of betaine in the composition ranges from between about 0.3 mM to about 0.7 mM. In yet other embodiments, the enhancer is betaine (or a derivative or analog thereof) and a concentration of betaine in the composition ranges from between about 0.4 mM to about 0.6 mM. In further embodiments, the enhancer is betaine (or a derivative or analog thereof) and a concentration of betaine in the composition is about 0.5 mM.

In yet other embodiments, combined release primer hybridization and extension may take place in the presence of a polymerase, betaine, and a mixture of unmodified and modified dNTPs. In these embodiments, the betaine is present at a concentration ranging from about 0.4 mM to about 0.6 mM; and where a ratio of the unmodified dNTPs to modified dNTPs is about 1:1. In some embodiments, the modified dNTPs are selected from 7-Deaza-2′-deoxyguanosine-5′-Triphosphates or 2-Amino-2′ deoxyadenosine-5′-Triphosphates.

In some embodiments, combined release primer hybridization and extension may take place in the presence of a polymerase, betaine, an optional mixture of unmodified and modified dNTPs, one or more buffers, and one or more divalent cations. In these embodiments, the betaine is present at a concentration ranging from about 0.4 mM to about 0.6 mM; and where a ratio of the unmodified dNTPs to modified dNTPs is about 1:1. In some embodiments, the betaine is present at a concentration ranging from about 0.4 mM to about 0.6 mM; a ratio of the unmodified dNTPs to modified dNTPs is about 1:1; and the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates. In some embodiments, the divalent cations are present at a concentration of about 0.1 mM to about 0.4 mM.

In some embodiments, combined release primer hybridization and extension may take place in the presence of a polymerase, a mixture of unmodified and modified dNTPs, one or more buffers, and one or more divalent cations. In these embodiments, a ratio of the unmodified dNTPs to modified dNTPs is about 1:1. In some embodiments, a ratio of the unmodified dNTPs to modified dNTPs is about 1:1; and the one or more divalent cations are present at a concentration of about 0.1 mM to about 0.4 mM. In some embodiments, the modified dNTPs are selected from 7-Deaza-2′-deoxyguanosine-5′-Triphosphates or 2-Amino-2′ deoxyadenosine-5′-Triphosphates.

In some embodiments, step 114 can further include termination of the primer extension reaction in order to control the length of the extended-release primer. Notably, the length of the extended-release primer product can be controlled actively through techniques such as inactivating the polymerase added in the step 114, or passively by enabling the reaction to go to completion such as through the consumption of limiting reactants or by controlling/selecting the size of the fragments of the nucleic acid molecules in the library of nucleic acid molecules.

In the case that the extended capture primer included one or more capture moieties attached to a solid support, liberation of the extended capture oligonucleotide in the step 114 results in a release primer extension complex that is free in solution as opposed to being attached to a solid support. Accordingly, as described in the step 110 of the method 100, one or more purification techniques can be implemented following the step 114 in order to recover the unbound second extension product or complex including the target nucleic acid from the support-attached extended capture primer, the second polymerase, other reaction components, the like, and combinations thereof.

The method 100 further includes a step 116 of post-capture amplification, involving linear or exponential amplification (e.g., PCR). In general, the step 116 includes amplifying the target nucleic acid with a third polymerase, a first amplification primer, and a second amplification primer. In some embodiments, the first and second amplification primers are designed to be complementary to the sequences of the adapters incorporated into the target nucleic acid molecules in the library of nucleic acid molecules in the step 102. For example, the first amplification primer can have a 3′ end complementary to the first adapter and the second amplification primer can have a 3′ end complementary to the second adapter. However, the primers for amplification can include any sequences that are present within the target nucleic acid being amplified (e.g., gene/target specific primers, universal primers, or the like) and can support synthesis of one or both strands (i.e., both the top and bottom strands of a double-stranded nucleic acid molecules corresponding to the template of the amplification reaction). In some embodiments, post-capture amplification may be conducted in a composition similar to those utilize in pre-capture amplification, including any of those compositions recited herein.

In some embodiments, the step 116 enables selective amplification of the target nucleic acid molecules from the library of nucleic acid molecules as opposed to amplification of either of the extended capture or release primers derived from the target nucleic acid. In one example, an uracil compatible polymerase and dUTP are included in one or both of the extension reactions carried out in the step 106 and the step 114. The extended amplification primers resulting from the reaction will include at least one uracil nucleotide, whereas the target nucleic acid template can be a DNA template having no uracil nucleotides. Thereafter, an uracil incompatible polymerase is included in the step 116 for amplification of the target nucleic acid. The uracil incompatible polymerase can amplify the target nucleic acid having no uracil nucleotides; however, the uracil incompatible polymerase will be incapable of replicating the uracil-containing extended oligonucleotide primers. Alternatively, or in addition, uracil-containing products can be selectively digested or otherwise degraded, thereby leaving behind only the original molecules from the library of nucleic acid molecules.

After the step 116 of amplification, the method 100 can include a step 118 of analyzing the amplified target nucleic acid molecules. The step 116 can include any method for determining the nucleic acid sequence of one or more products of the method 100. The step 116 can further include sequences alignment, identification of sequence variations, counting of unique primer extension products, the like, or combinations thereof.

In some embodiments, amplicons generated after step 116 are sequenced, such as utilizing next generation sequencing. As used herein, the term “next generation sequencing” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25-about 500 bp) but many hundreds of thousands or millions of reads in a relatively short time. Examples of such sequencing devices available from Illumina (San Diego, CA) include, but are not limited to iSEQ, MiniSEQ, MiSEQ, NextSEQ, NoveSEQ. It is believed that the Illumina next-generation sequencing technology uses clonal amplification and sequencing by synthesis (SBS) chemistry to enable rapid sequencing. The process simultaneously identifies DNA bases while incorporating them into a nucleic acid chain. Each base emits a unique fluorescent signal as it is added to the growing strand, which is used to determine the order of the DNA sequence. A non-limiting example of a sequencing device available from ThermoFisher Scientific (Waltham, MA) includes the Ion Personal Genome Machine™ (PGM™) System. It is believed that Ion Torrent sequencing measures the direct release of H+ (protons) from the incorporation of individual bases by DNA polymerase. A non-limiting example of a sequencing device available from Pacific Biosciences (Menlo Park, CA) includes the PacBio Sequel Systems. A non-limiting example of a sequencing device available from Roche (Pleasanton, CA) is the Roche 454. Next-generation sequencing methods may also include nanopore sequencing methods. In general, three nanopore sequencing approaches have been pursued: strand sequencing in which the bases of DNA are identified as they pass sequentially through a nanopore, exonuclease-based nanopore sequencing in which nucleotides are enzymatically cleaved one-by-one from a DNA molecule and monitored as they are captured by and pass through the nanopore, and a nanopore sequencing by synthesis (SBS) approach in which identifiable polymer tags are attached to nucleotides and registered in nanopores during enzyme-catalyzed DNA synthesis. Common to all these methods is the need for precise control of the reaction rates so that each base is determined in order. Strand sequencing requires a method for slowing down the passage of the DNA through the nanopore and decoding a plurality of bases within the channel; ratcheting approaches, taking advantage of molecular motors, have been developed for this purpose. Exonuclease-based sequencing requires the release of each nucleotide close enough to the pore to guarantee its capture and its transit through the pore at a rate slow enough to obtain a valid ionic current signal. In addition, both of these methods rely on distinctions among the four natural bases, two relatively similar purines and two similar pyrimidines. The nanopore SBS approach utilizes synthetic polymer tags attached to the nucleotides that are designed specifically to produce unique and readily distinguishable ionic current blockade signatures for sequence determination. In some embodiments, sequencing of nucleic acid molecules comprises via nanopore sequencing comprises preparing nanopore sequencing complexes and determining polynucleotide sequences. Methods of preparing nanopores and nanopore sequencing are described in U.S. Patent Application Publication No. 2017/0268052, and PCT Publication Nos. WO2014/074727, WO2006/028508, WO2012/083249, and WO/2014/074727, the disclosures of which are hereby incorporated by reference herein in their entireties. In some embodiments, tagged nucleotides may be used in the determination of the polynucleotide sequences (see, e.g., PCT Publication No. WO/2020/131759, WO/2013/191793, and WO/2015/148402, the disclosures of which are hereby incorporated by reference herein in their entireties). Analysis of the data generated by sequencing is generally performed using software and/or statistical algorithms that perform various data conversions, e.g., conversion of signal emissions into base calls, conversion of base calls into consensus sequences for a nucleic acid template, etc. Such software, statistical algorithms, and the use of such are described in detail, in U.S. Patent Application Publication Nos. 2009/0024331 2017/0044606 and in PCT Publication No. WO/2018/034745, the disclosures of which are hereby incorporated by reference herein in their entireties.

### EXAMPLES

### Example 1—Effect of Various Primer Maximum Melting Temperature (Tm) Limits on the Enrichment of High % GC Targets

### Materials

NA12878 cell line DNA
Hot Spot Panel
HyperPlus Library Preparation Kit
KAPA UDI Primer Mixes
KAPA Universal UMI Adapters

### Method

Panels were designed to three high % GC targets using no limits on % GC, a minimum Tm of 57° C. and various max Tm limits (e.g., 66° C., 69° C., 72° C., no limit).

Twenty-four libraries were made according to the KAPA HyperPETE Somatic Tissue DNA Workflow v1.0 Chapter 4. Library Preparation from DNA using 10 ng of non-formalin compromised NA24143 cell line DNA with exceptions listed as follows: During Step 5, Post-Ligation 0.8× Purification with KAPA HyperPure Beads, adapter ligated DNA samples were pooled and the pool aliquoted into 20 μL per sample going into Step 6.

Primer extension target enrichment was performed on the ten libraries according to the KAPA HyperPETE Somatic Tissue DNA Workflow v1.0 Chapter 5 (the disclosure of which is hereby incorporated by reference herein in its entirety). Primer Extension Target Enrichment (PETE) (described herein) was performed with exceptions listed as follows: During Step 2, Capture Extension Reaction, 5 μL of five small custom capture test panels targeting three high % GC ROIs using various max Tm limits, Table 8, was added to four samples per condition. 5 μL of Hot Spot Capture Panel was added, but no water was included in the Capture Extension Reaction Master Mix. During Step 6, Release Primer Hybridization, 20 μL instead of 30 μL of 1× Wash & Resuspension Buffer and 10 μL of Hot Spot Release Panel was added to the Release Primer Hybridization Master Mix. 10 μL of five test capture panels were added to corresponding four samples per condition.

Primer Extension Target Enrichment libraries were sequenced on a NextSeq 550 and analyzed. Normalized coverage at each position for each sample was calculated by dividing the coverage at a position by the average coverage for the whole sample.

TABLE 8
Experimental setup.

| | Current Primer | | | | |
|---|---|---|---|---|---|
| | Database | MaxTm66 | MaxTm69 | MaxTm72 | NoMaxTm |
| Tm | 57 °C - | 57 °C - | 57 °C - | 57 °C - | 57 °C - no |
| Limits | 63 °C | 66 °C | 69 °C | 72 °C | limit |
| % GC | 20% - 80% | any | any | any | any |
| Limits | | | | | |

### Results

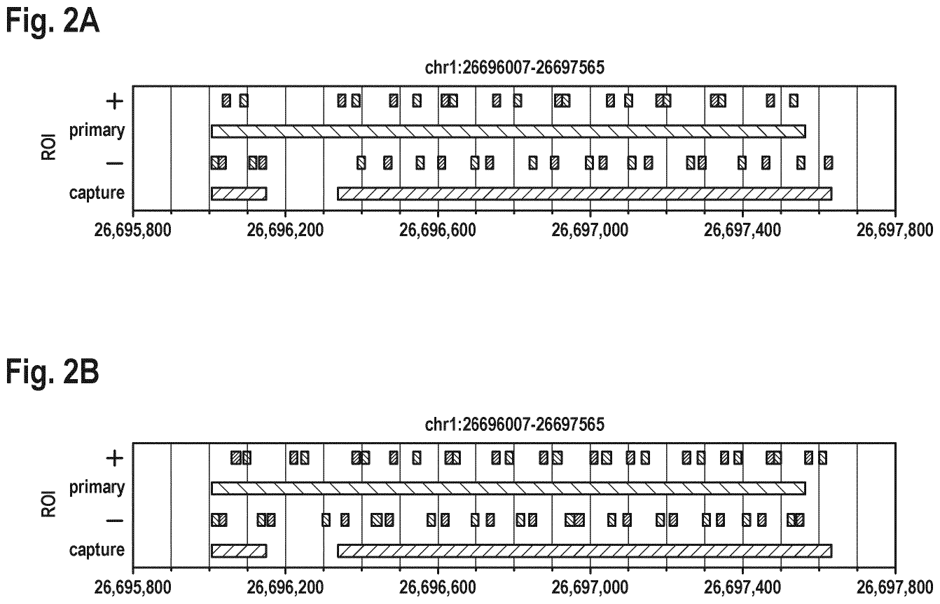

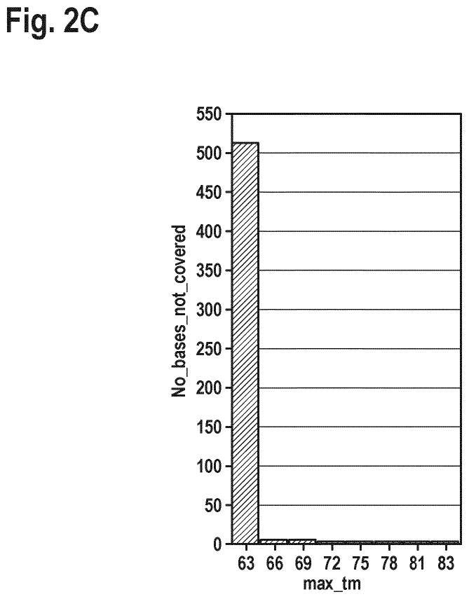

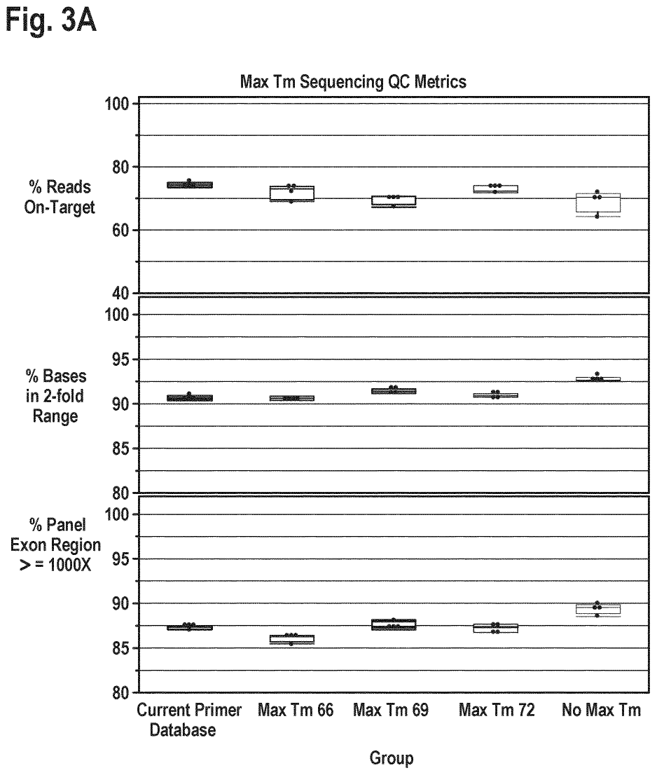

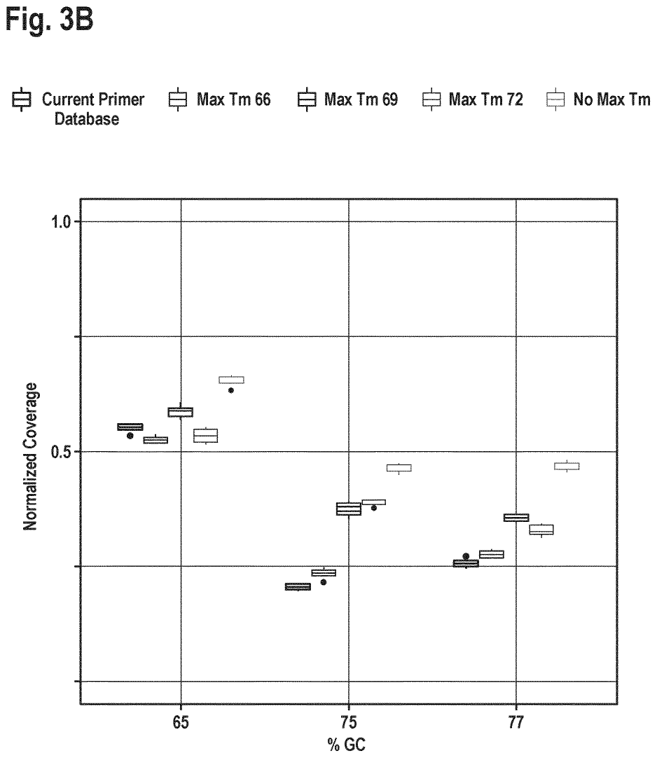

The results for the current primer database are illustrated in FIG. 2A. Likewise, the results for the “no Max Tm Panel” are illustrated in FIG. 2B. FIG. 2C depicts primer availability. Increasing the primer max Tm increased the availability of primers in high % GC regions (a) and reduced the number of targeted bases not covered by capture and release primer spans (b). As shown in FIG. 3A, samples enriched with the No Max Tm test panel had the highest percent bases in 2-fold range, and percent panel exon region >=1000×. It was found that higher primer max Tm improved normalized coverage of targets >65% GC compared to the current primer database with a max Tm of 57° C. (see FIG. 3D). The small custom panel designed with no max primer Tm had the highest normalized coverage in high % GC targets.

### Conclusion

Applicant has found that target enrichment in the presence of primers which were synthesized with no restrictions placed on melting temperature and/or GC content improved the enrichment of high GC target nucleic acid molecules, ultimately resulting in improved uniformity of coverage when the high GC target enriched nucleic acid molecules were sequenced.

### Example 2-Effect of 7-Deaza DGTP and Betaine in PCR and Capture Extension

### Purpose

Test the effects of adding 7-deaza dGTP to library preparation amplification and betaine to both library preparation amplification and capture extension reaction on the enrichment of high % GC targets.

### Materials

NA24143 cell line DNA
Hot Spot Panel
Small Custom Panel (3 High % GC ROIs)
HyperPlus Library Preparation Kit
KAPA UDI Primer Mixes
KAPA Universal UMI Adapters

### Method

Sixteen libraries were made according to the KAPA HyperPETE Somatic Tissue DNA Workflow v1.0 Chapter 4 (the disclosure of which is hereby incorporated by reference herein in its entirety). Library Preparation from DNA using 10 ng of non-formalin compromised NA12878 cell line DNA with exceptions listed as follows: During Step 5, Post-Ligation 0.8× Purification with KAPA HyperPure Beads, 12 μL of 10 mM Tris-HCl pH 8.0 was used to elute the adapter-ligated DNA sample rather than the Instructions for Use instructed 20 μL. Adapter ligated DNA samples were pooled and the pool aliquoted into 12 μL per sample going into Step 6, During Step 6 Amplification with KAPA UDI Primer Mixes (also referred to as PCR1), 3 μL of water was added to eight samples and 3 μL of 5 mM 7-deaza dGTP was added to the other eight samples. Then within each set of eight samples, 5 μL of water was added to four samples and 5 μL of 5M betaine was added to the other four samples. During Step 7, Post-amplification 1× Purification with KAPA HyperPure Beads, the pre-capture libraries were eluted with 15 μL of 10 mM Tris-HCl pH 8.0 rather than the 25 μL instructed in the Instructions for Use.

Primer extension target enrichment was performed on the sixteen libraries according to the KAPA HyperPETE Somatic Tissue DNA Workflow v1.0 Chapter 5, Primer Extension Target Enrichment (PETE) with exceptions listed as follows: During Step 2, Capture Extension Reaction, 5 μL of each pre-capture input library was used instead of the instructed 10-15 μL which resulted in less than 500 ng of pre-capture input library per sample for some samples. In addition to 5 μL of Hot Spot Capture Panel, 5 μL of a small custom capture panel targeting three high % GC ROIs was added to the Capture Extension Reaction Master Mix. No water was included in the Capture Extension Reaction Master Mix, but rather 5 μL of either water or 5M Betaine was added to the 5 μL of pre-capture input libraries such that each combination of conditions had two replicates (Table 9). During Step 6, Release Primer Hybridization, 20 μL of 1× Wash & Resuspension Buffer was added to the Release Primer Hybridization Master Mix instead of 30 μL and 10 μL of a small custom release panel was added along with 10 μL of Hot Spot Release Panel.

Primer Extension Target Enrichment libraries were sequenced on a NextSeq 550 and analyzed. Normalized coverage at each position for each sample was calculated by dividing the coverage at a position by the average coverage for the whole sample.

TABLE 9
Experimental setup.

| | PCR1 - 7-deaza | PCR1 + 7-deaza | |
|---|---|---|---|
| | dGTP | dGTP | |
| PCR1 - | UDI-P 1 | UDI-P 9 | Capture Extension |
| Betaine | | | -Betaine |
| | UDI-P 2 | UDI-P 10 | |
| | UDI-P 3 | UDI-P 11 | Capture Extension |
| | UDI-P 4 | UDI-P 12 | +Betaine |
| PCR1 + | UDI-P 5 | UDI-P 13 | Capture Extension |
| Betaine | | | -Betaine |
| | UDI-P 6 | UDI-P 14 | |
| | UDI-P 7 | UDI-P 15 | Capture Extension |
| | | | +Betaine |
| | UDI-P 8 | UDI-P 16 | |

### Results

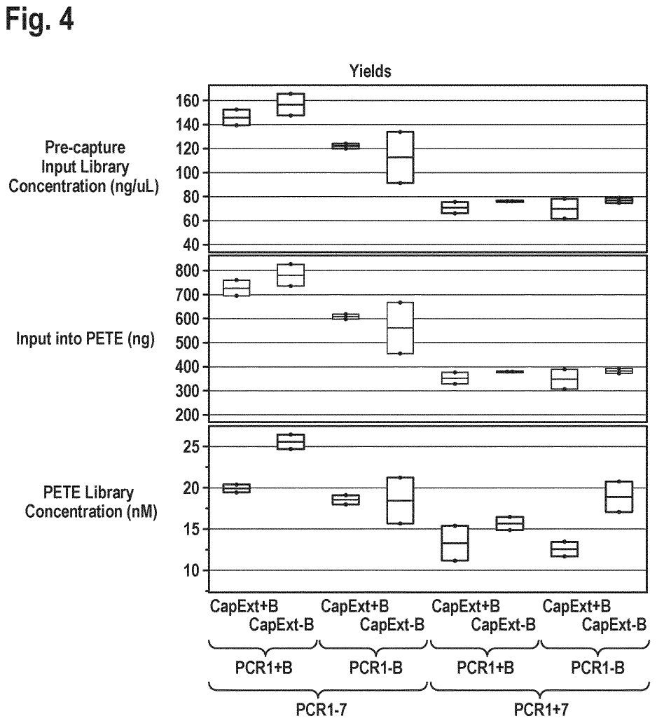

Concentrations of pre-capture input libraries were lower for samples with 7-deaza dGTP added (PCR1+7) compared to samples without (PCR1−7) (see FIG. 4). Due to the low pre-capture input library yield, input into PETE was also lower for samples with 7-deaza dGTP. PETE library yields were lower for samples with betaine in the capture extension reaction (CapExtx+B) compared to samples without betaine in capture extension with shared PCR1 conditions (see FIG. 4).

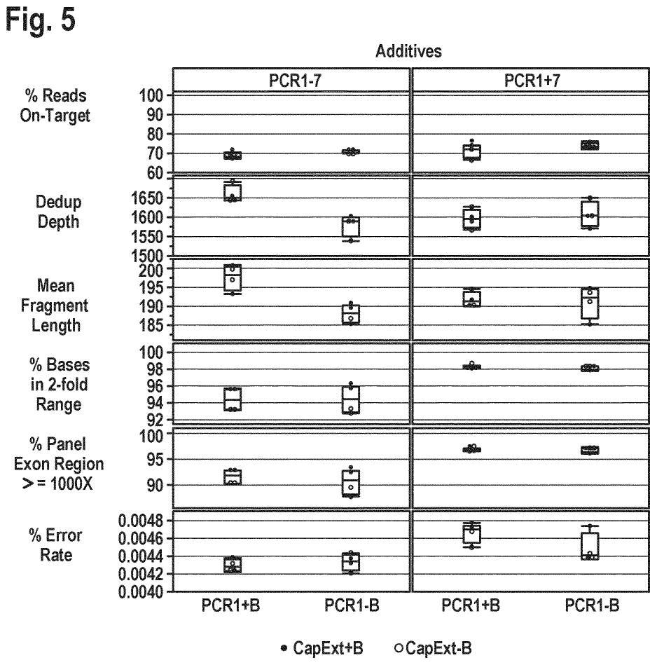

Percent reads on target were similar between all test conditions. Dedup depth was higher and mean fragment length was larger for samples with betaine in PCR1. Percent bases in 2-fold range, percent panel exon region >=1000×, and percent error rates were higher for samples with 7-deaza dGTP. For samples without 7-deaza dGTP, samples with betaine in the capture extension reaction had higher percent bases in 2-fold range and percent exon region >=1000× (see FIG. 5).

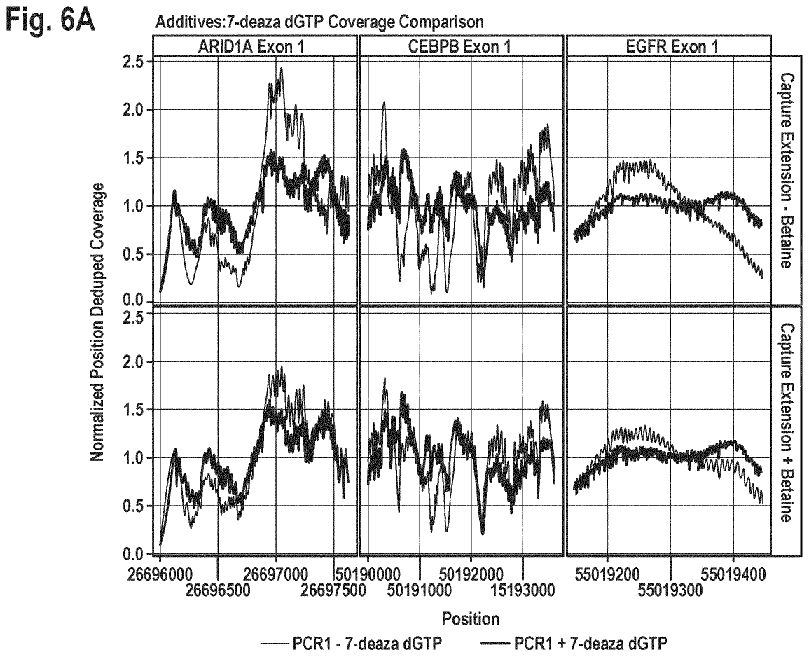

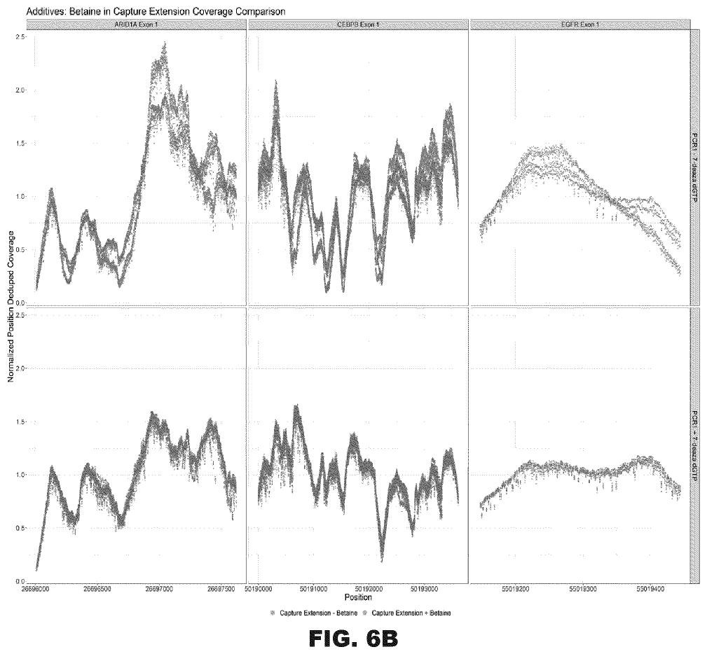

FIGS. 6A and 6B show the normalized Coverage across High % GC targets. In particular, FIG. 6A depicts that coverage is more even across three high % GC ROIs targeted by the small custom panel for samples with 7-deaza dGTP than samples without 7-deaza dGTP. FIG. 6B illustrates that for samples without 7-deaza dGTP, coverage is more even across three high % GC ROIs targeted by the small custom panel for samples with betaine in the capture extension reaction than samples without betaine in the capture extension reaction.

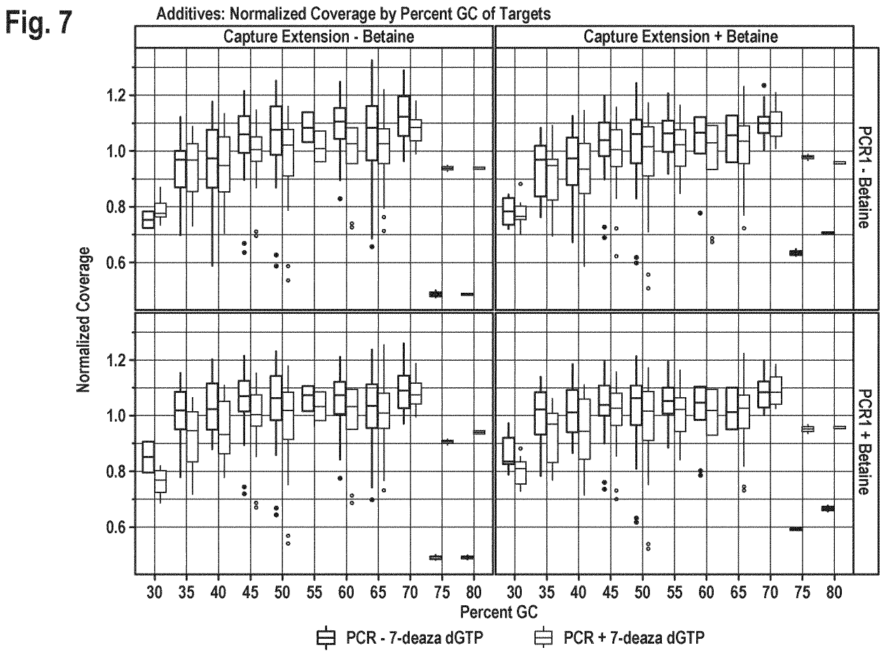

FIG. 7 illustrates the normalized Coverage of Targets by % GC. Normalized coverage of targets with >70% GC is higher for samples with 7-deaza dGTP than samples without 7-deaza dGTP. For samples without 7-deaza dGTP, normalized coverage of targets with >70% GC is higher with betaine in the capture extension reaction than samples without betaine in the capture extension reaction.

### Conclusion

Applicant has found that the addition of either 7-deaza dGTP in library preparation or betaine in capture extension can be used to improve high % GC target enrichment. Further testing is needed to determine the variant calling effects of higher percent error rates seen in samples with 7-deaza dGTP.

### Example 3—Capture Extension Program Optimizations

### Purpose

Test the effect of different capture extension incubation times and temperatures on the enrichment of high % GC targets.

### Materials

NA24143 cell line DNA
Hot Spot Panel
Small Custom Panel (3 High % GC ROIs)
HyperPlus Library Preparation Kit
KAPA UDI Primer Mixes
KAPA Universal UMI Adapters

### Method

Ten libraries were made according to the KAPA HyperPETE Somatic Tissue DNA Workflow v1.0 Chapter 4, Library Preparation from DNA using 10 ng of non-formalin compromised NA24143 cell line DNA with exceptions listed as follows: During Step 5, Post-Ligation 0.8× Purification with KAPA HyperPure Beads, adapter ligated DNA samples were pooled and the pool aliquoted into 20 μL per sample going into Step 6.

Primer extension target enrichment was performed on the ten libraries according to the KAPA HyperPETE Somatic Tissue DNA Workflow v1.0 Chapter 5 (the disclosure of which is hereby incorporated by reference herein in its entirety). Primer Extension Target Enrichment (PETE) with exceptions listed as follows: During Step 2, Capture Extension Reaction, 5 μL of Hot Spot Capture Panel, 5 μL of a small custom capture panel targeting three high % GC ROIs was added to the Capture Extension Reaction Master Mix. No water was included in the Capture Extension Reaction Master Mix. Two samples were incubated in each of four test capture extension programs detailed in Table 10 or a control capture extension program according to the thermocycler program in Step 2.2. During Step 6, Release Primer Hybridization, 20 μL of 1× Wash & Resuspension Buffer was added to the Release Primer Hybridization Master Mix instead of 30 μL and 10 μL of a small custom release panel was added along with 10 μL of Hot Spot Release Panel.

Primer Extension Target Enrichment libraries were sequenced on a NextSeq 550 and analyzed. Normalized coverage at each position for each sample was calculated by dividing the coverage at a position by the average coverage for the whole sample

TABLE 10
Capture programs tests.

| Test Program 1 | | | Test Program 2 | | | Test Program 3 | | | Test Program 4 | | | Control | | |
|---|---|---|---|---|---|---|---|---|---|---|---|---|---|---|
| Temp. | Time | | Temp. | Time | | Temp. | Time | | Temp. | Time | | Temp. | Time | |
| 95 °C | 2 | min. | 95 °C | 2 | min. | 95 °C | 2 | min. | 95 °C | 2 | min. | 95 °C | 2 | min. |
| 80 °C | 3 | min. | 80 °C | 1 | min. | 80 °C | 1 | sec. | 80 °C | 1 | sec. | 80 °C | 1 | sec. |
| 75 °C | 4.5 | min. | 75 °C | 5 | min. | Slow | Not | | Slow | Not | | Slow | 10 | min. |
| | | | | | | ramp | measured | | ramp | measured | | ramp | | |
| 80 °C | 4.5 | min. | 70 °C | 5 | min. | 75 °C | 10 | min. | 72 °C | 10 | min. | 60 °C | 10 | min. |
| 65 °C | 4.5 | min. | 60 °C | 10 | min. | 70 °C | 10 | min. | 66 °C | 10 | min. | 65 °C | 2 | min. |
| 60 °C | 4.5 | min. | 65 °C | 2 | min. | 60 °C | 10 | min. | 60 °C | 10 | min. | Total: | ~24 | min. |
| 65 °C | 2 | min. | Total: | ~25 | min. | 65 °C | 2 | min. | 65 °C | 2 | min. | | | |
| Total: | ~25 | min. | | | | Total: | ~34 | min. | Total: | ~34 | min. | | | |

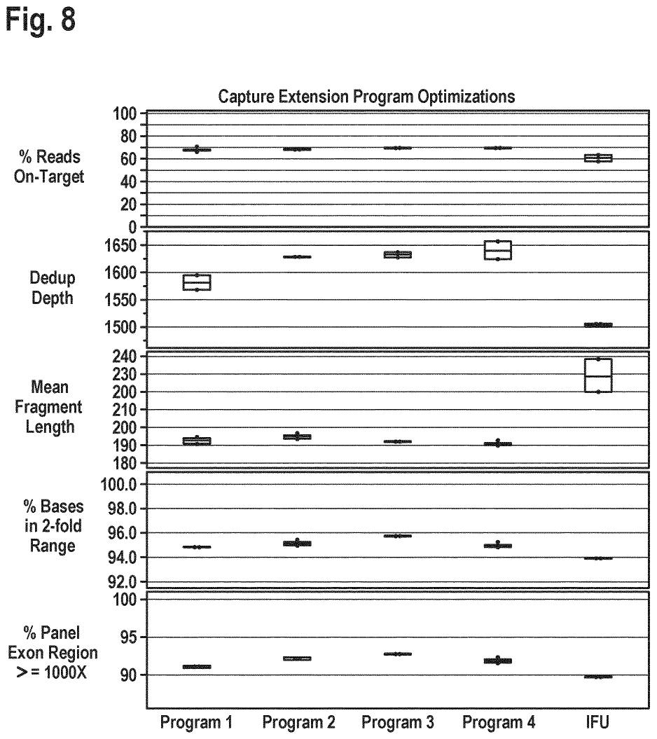

FIG. 8 depicts sequencing quality control metrics. Percent reads on target, dedup depth, percent bases in 2-fold range, and percent panel exon region >=1000× were higher for the test programs 1~4 samples compared to the program indicated in the Instructions for Use (IFU) samples. Mean fragment length was higher for the IFU program. Program 3 samples had the highest percent bases in 2-fold range and percent panel exon region >=1000×.

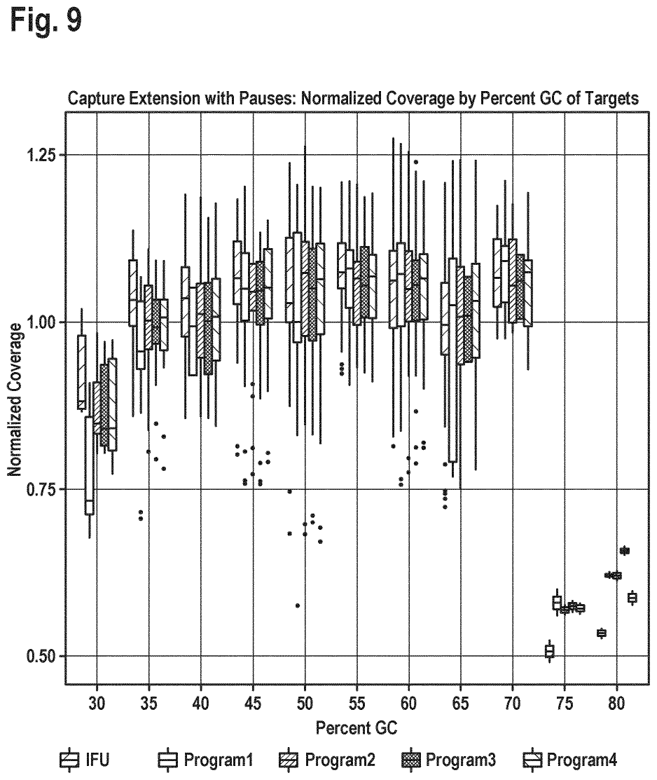

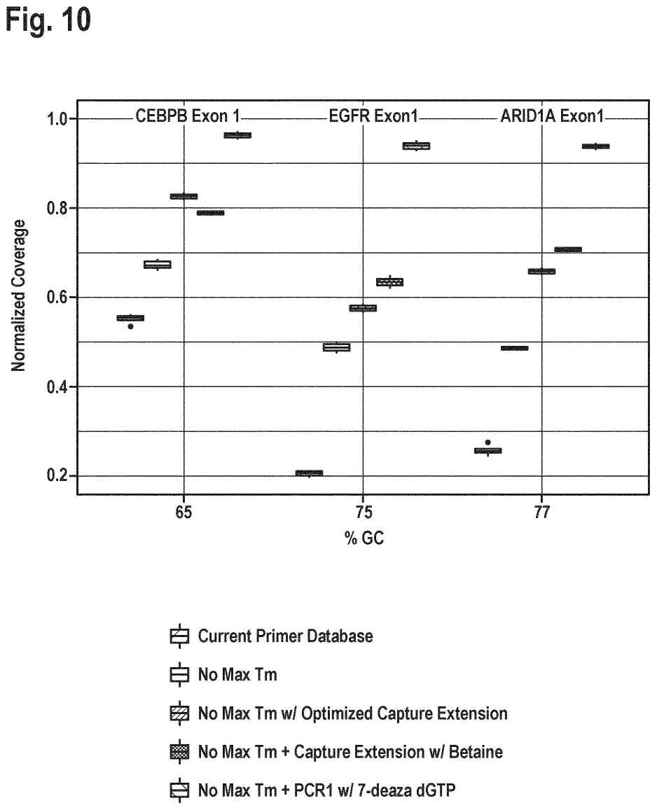

FIG. 9 depicts normalized coverage per target. Normalized coverage of targets with >70% GC is higher for test programs 1-4 samples compared to IFU samples with program 3 samples having the highest normalized coverage for targets >75% GC. Test programs 1-4 samples had lower normalized coverage in targets <40% GC compared with IFU samples with program 1 samples having the lowest coverage in targets <35% GC.

### Conclusion

Test program 3 (Table 10) with longer incubation times at higher temperatures produced the biggest improvements in normalized coverage of high % GC targets, percent bases in 2-fold range, and percent panel exon region >=1000× compared to other test programs and the IFU program. Applicant has found that longer incubation times and higher temperatures may result in improved normalized coverage of high % GC targets.

### Example 4—Effect of 7-Deaza DGTP, 2-Amino DATP, DMSO, Trehalose, Betaine, and Single Stranded Binding Protein on Capture Extension

The purpose of this study was to test the effects of adding 7-deaza dGTP and 2-amino dATP to library preparation amplification as well as DMSO, Trehalose, Betaine, and single stranded DNA binding protein to the capture extension reaction on the enrichment of high % GC targets.

### Materials

NA12878 Cell Line DNA
No Max Tm Panel
KAPA HyperPETE Hotspot Panel
2-amino dATP
7-deaza dGTP
Single stranded DNA Binding Protein
Betaine
Trehalose
DMSO
KAPA HyperPETE Reagent Kit
KAPA HyperCapture Bead Kit
KAPA HyperPlus Library Preparation Kit
KAPA UDI Primer Mixes
KAPA Universal UMI Adapters

### Methods

Sixteen libraries were made according to the KAPA HyperPETE Somatic Tissue DNA Workflow v1.0 Chapter 4. Library Preparation from DNA using 10 ng of non-formalin compromised NA24143 cell line DNA with exceptions listed as follows: During Step 5, Post-Ligation 0.8× Purification with KAPA HyperPure Beads, adapter ligated DNA samples were pooled and the pool aliquoted into 20 μL per sample going into Step 6. In four replicates, 2-amino-dATP was added at 0.3 mM concentration to the PCR amplification (PCR1). In another four replicates, 2-amino-dATP and 7-deaza-dGTP were added at 0.3 mM concentration each to the PCR1 amplification. In another four replicates, 7-deaza-dGTP was added at 0.3 mM concentration to the PCR1 amplification (Table 11). In the last four replicates, no additives were included in the PCR1 amplification.

Primer extension target enrichment was performed on 10 μL of each of the 16 libraries according to the KAPA HyperPETE Somatic Tissue DNA Workflow v1.0 Chapter 5. Primer Extension Target Enrichment (PETE) with exceptions listed as follows: During Step 2. Capture Extension Reaction, equal concentrations of No MaxTm high % GC ROI capture panel Hot Spot Capture Panel were added to the Capture Extension Reaction Master Mix. During Step 6, Release Primer Hybridization, the volume of 1× Wash & Resuspension Buffer was reduced to accommodate equal concentrations of the Hot Spot Release Panel and the No Max Tm release panel in the Release Primer Hybridization Master Mix.

Twenty-four libraries were made according to the KAPA HyperPETE Somatic Tissue DNA Workflow v1.0 Chapter 4, Library Preparation from DNA using 10 ng of non-formalin compromised NA24143 cell line DNA with exceptions listed as follows: During Step 5, Post-Ligation 0.8× Purification with KAPA HyperPure Beads, adapter ligated DNA samples were pooled and the pool aliquoted into 20 μL per sample going into Step 6.

Primer extension target enrichment was performed on 10 μL of each of the 16 libraries according to the KAPA HyperPETE Somatic Tissue DNA Workflow v1.0 Chapter 5, Primer Extension Target Enrichment (PETE) with exceptions listed as follows: During Step 2, Capture Extension Reaction, equal concentrations of the No MaxTm high % GC ROI capture panel and of Hot Spot Capture Panel were added to the Capture Extension Reaction Master Mix. Single stranded DNA binding protein at 100 ng, 200 ng, or 400 ng amounts were added to three replicates each. Betaine was added to three replicates to achieve a 0.5M final concentration. Trehalose was added to three replicates to achieve a 0.1M final concentration. DMSO was added at a 5% or 10% final concentration to three replicates each (Table 111). Three replicates had no capture extension additives. During Step 6, Release Primer Hybridization, the volume of 1× Wash & Resuspension Buffer was reduced to accommodate equal concentrations of the Hot Spot Release Panel and the No Max Tm release panel in the Release Primer Hybridization Master Mix.

Primer Extension Target Enrichment libraries were sequenced on a NextSeq 500 and analyzed using a Roche internal pipeline with 8M total subsampled reads. Normalized coverage at each position for each sample was calculated by dividing the coverage at a position by the average coverage for the whole sample.

TABLE 11
Experimental setup.

| | Final | |
|---|---|---|
| Additive | Concentration | Replicates |
| 2-amino-dATP | 0.3 mM | 4 |
| Both 2-amino-dATP and 7-deaza-dGTP | 0.3 mM each | 4 |
| 7-deaza-dGTP | 0.3 mM | 4 |
| Single Stranded DNA Binding Protein | 2 ng/μL | 3 |
| (100 ng total) | | |
| Single Stranded DNA Binding Protein | 4 ng/μL | 3 |
| (200 ng total) | | |
| Single Stranded DNA Binding Protein | 8 ng/μL | 3 |
| (400 ng total) | | |
| Betaine | 0.5 M | 3 |
| Trehalose | 0.1 M | 3 |
| DMSO | 10% | 3 |
| DMSO | 5% | 3 |

### Results

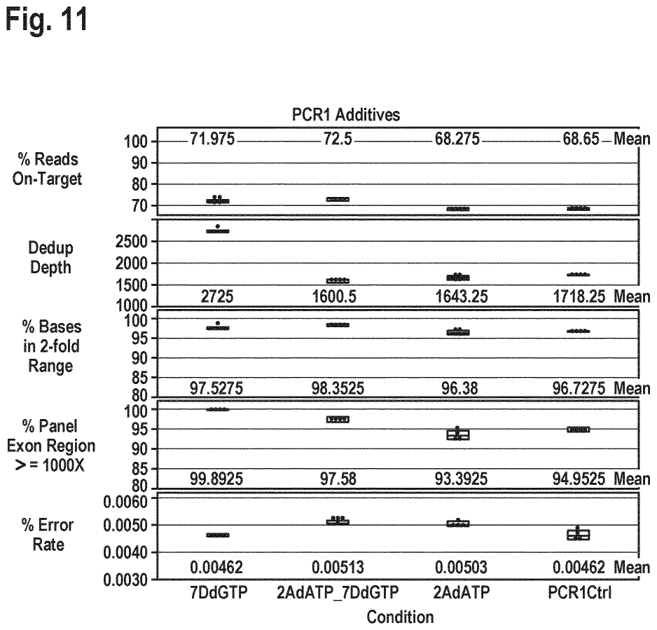

Percent reads on target were similar between 7-deaza dGTP (7DdGTP) only samples and 7DdGTP and 2-amino dATP (2AdATP) samples, which were slightly higher than control samples (see FIG. 11). Control samples and 2AdATP only samples had similar percent reads on target. Samples with added 7DdGTP only had much higher dedup depth than control samples while samples with added 2AdATP, with or without 7DdGTP, had lower dedup depth than the control samples. Samples with 7DdGTP added, with or without 2AdATP, had slightly higher percent bases in 2-fold range than control samples and 2AdATP only samples. Samples with 7DdATP had much higher percent panel exon region greater than or equal to 1000× than control samples, followed by 2AdATP and 7DdATP samples. Samples with added 2AdATP only had slightly lower percent panel exon region greater than or equal to 1000× compared to control samples. Samples with added 7DdATP only had the same percent error rate as control samples while samples with 2AdATP added, with or without 7DdATP, had higher percent error rate compared to control samples.

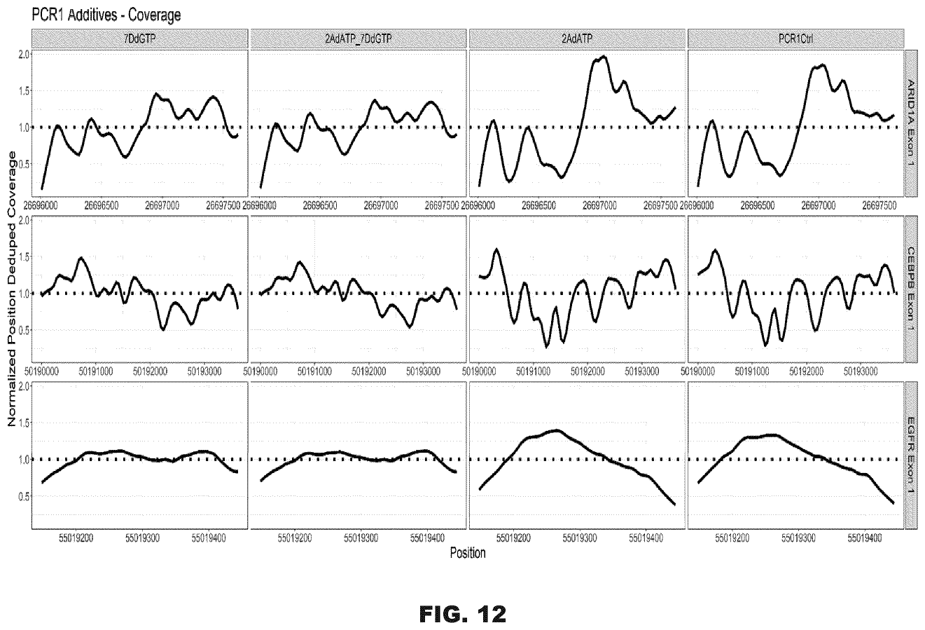

Normalized position deduped coverage across the three high percent GC targets in the No Max Tm panel was more even and close to the targeted normalized coverage of 1, as indicated with the dotted lines, for samples with 7DdTP added, with or without 2AdATP, compared to samples with only 2AdATP added and control samples (FIG. 12).

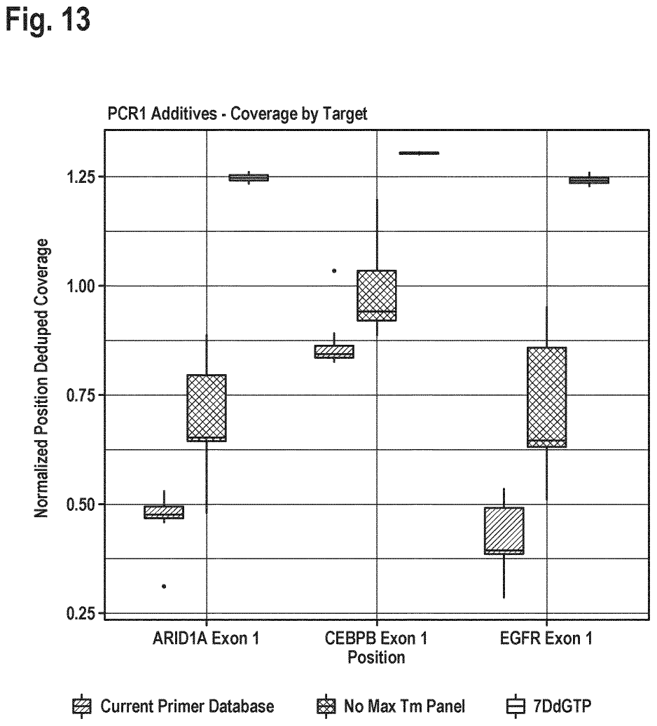

Normalized position deduped coverage was higher in high percent GC targets for samples captured with the No Max Tm Panel than for samples captured with the panel designed with the current primer database from a previous experiment; however, the median normalized coverage level was still under the ideal of 1 (see FIG. 13). With the addition of 7DdGTP in PCR1, normalized coverage for the three targets is above 1.

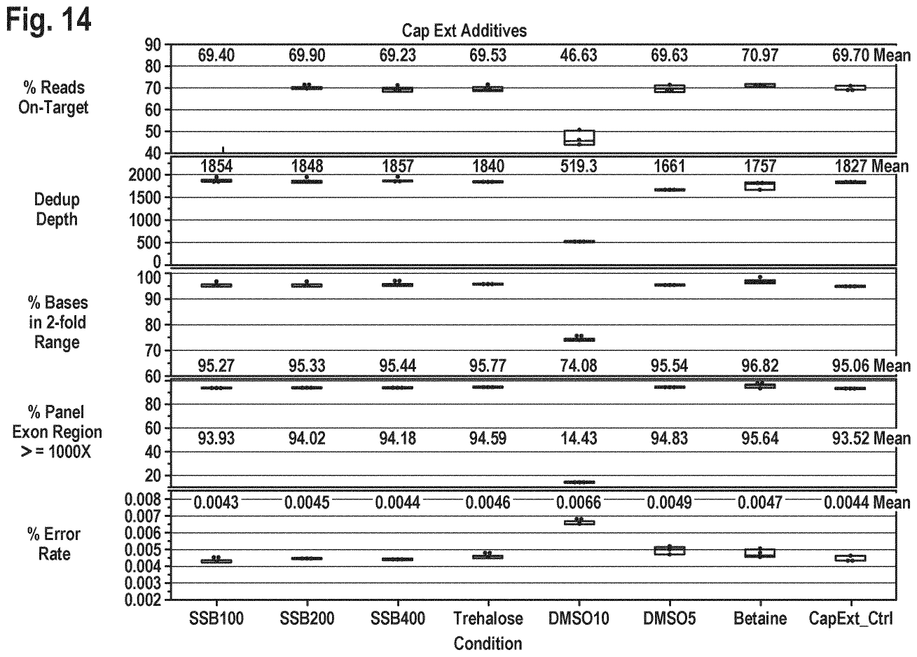

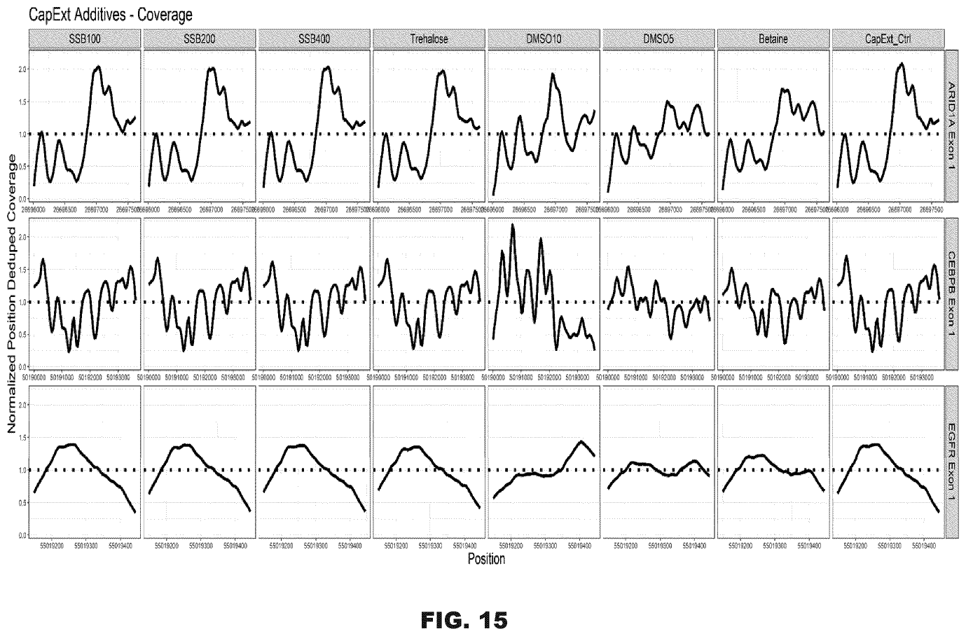

Percent reads on target was greatly decreased by the addition of 10% DMSO (DMSO10) and slightly increased by the addition of Betaine compared to all other samples which had similar percent reads on target (see FIG. 14). Dedup depth was greatly decreased by the addition of DMSO10 compared to all other samples which had similar dedup depths. Percent bases in 2-fold range and percent panel exon region greater than or equal to 1000× were greatly decreased by the addition of DMSO10 compared to all other samples which had similar performance. Percent error rate increased by the addition of DMSO10 compared to all other samples which had similar percent error rates.

Normalized position deduped coverage across the three high percent GC targets in the No Max Tm panel was more even and close to the targeted normalized coverage of 1, as indicated with the dotted lines, for samples with DMSO5 or Betaine added compared to control samples (see FIG. 15). Samples with DMSO10 added had the opposite profile for CEBPB Exon 1 and EGFR Exon 1 compared to control samples as in high coverage portions for control samples were low coverage in DMSO10 samples and vice versa.

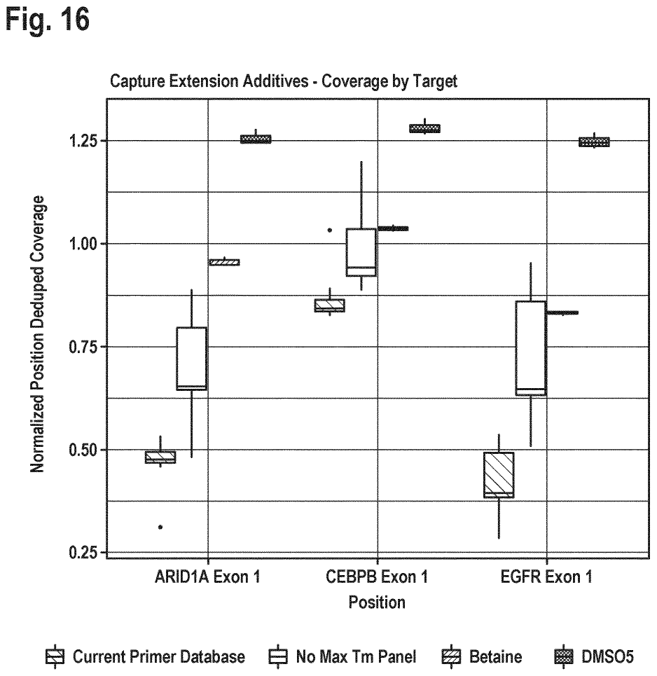

Normalized position deduped coverage was higher in high percent GC targets for samples captured with the No Max Tm Panel than for samples captured with the panel designed with the current primer database from a previous experiment; however, the median normalized coverage level was still under the ideal of 1 (see FIG. 16). With the addition of Betaine in Capture Extension, normalized coverage for the three targets is closer to the ideal of 1 compared to samples with no additions (No Max Tm Panel). With the addition of DMSO5 in Capture Extension, normalized coverage for the three targets is well above the ideal of 1.

### Conclusion

The addition of 7-deaza dGTP into PCR1, Betaine in Capture Extension, and 5% DMSO in Capture Extension all improve the coverage in high percent GC target regions. The addition of 7-deaza dGTP also improved many sequencing QC metrics. The addition of Betaine produced similar performance in sequencing QC metrics compared to no additives, while the addition of 5% DMSO showed some minor decreases in sequencing QC metric performance.

### Example 6—Titration of DMSO in Capture Extension

### Purpose

The purpose of this study was to perform a titration of DMSO in the capture extension using a large 1.7 Mb panel and evaluate the impact on sequencing QC metrics and coverage of high % GC targets.

### Materials

NA12891 Cell line DNA
1.7 Mb Panel
DMSO
KAPA HyperPETE Reagent Kit
KAPA HyperCapture Bead Kit
KAPA HyperPlus Library Preparation Kit
KAPA UDI Primer Mixes
KAPA Universal UMI Adapters
High Sensitivity D1000 Reagents
NextSeq500/550 HO Reagents

### Methods

Eighteen libraries were prepared from 10 ng of non-formalin compromised NA12891 Cell line DNA according to the KAPA HyperPETE Somatic Tissue DNA Workflow v1.0 Chapter 4, with the exception that libraries were pooled post-ligation clean-up and re-aliquoted in 20 μL volumes to limit sample to sample library preparation processing differences. The KAPA HyperPETE Somatic Tissue DNA Workflow v1.0 Chapter 5 was followed for enrichment, with some notable modifications to facilitate using a custom-made 1.7 Mb HyperPETE panel, which is outside the size range for on-market HyperPETE panels. The 1.7 Mb panel was designed using an expanded primer database that included % GC from 0-100 and Tm of 57-100° C.

Library input to Primer Extension Target Enrichment (PETE) was normalized to 1000 ng of library per sample. The PETE workflow modifications included diluting the capture primers to a 0.37× per primer concentration relative to the standard 1× per primer concentration, therefore keeping the total capture primer concentration at 2× the concentration of the largest on-market panel, the HyperPETE Pan Cancer Panel. Other modifications included adding twice as much Human Cot DNA in the capture extension by using a 3× HyperPure Bead Clean-up to concentrate the Cot DNA and using 300 μL of capture beads versus 100 μL in the standard protocol. At the capture extension step, master mixes were made to include DMSO from 0-5% (Table 12). For the PCR amplification step, 13 cycles were used based on the panel size. PETE libraries were sequenced on a NextSeq 500 and analyzed using a Roche internal pipeline with subsampling at 60M total reads/sample.

TABLE 12
Experimental setup.

| Panel | % DMSO in capture extension | Replicates |
|---|---|---|
| 1.7 Mb Custom | 0% | 3 |
| HyperPETE | 1% | 3 |
| Panel | 2% | 3 |
| | 3% | 3 |
| | 4% | 3 |
| | 5% | 3 |

### Results

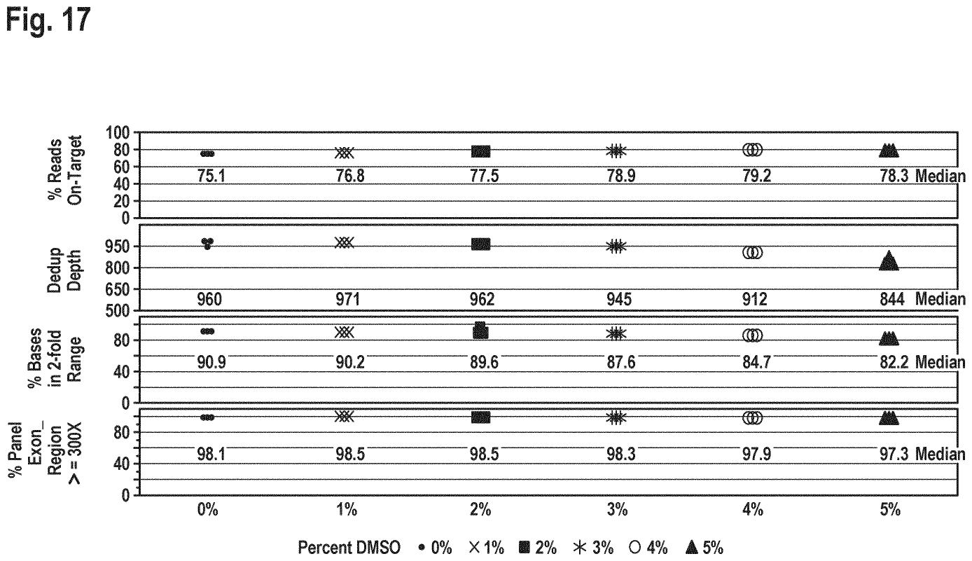

On-target rate increased with increasing amounts of DMSO in the capture extension (see FIG. 17). Dedup depth remained similar up to 2% DMSO and then began to decrease. For uniformity, % bases in 2-fold range decreased with increasing amounts of DMSO. The % panel exon region greater than or equal to 300× metric was best at 1% and 2% DMSO and began to decrease with increasing amounts of DMSO.

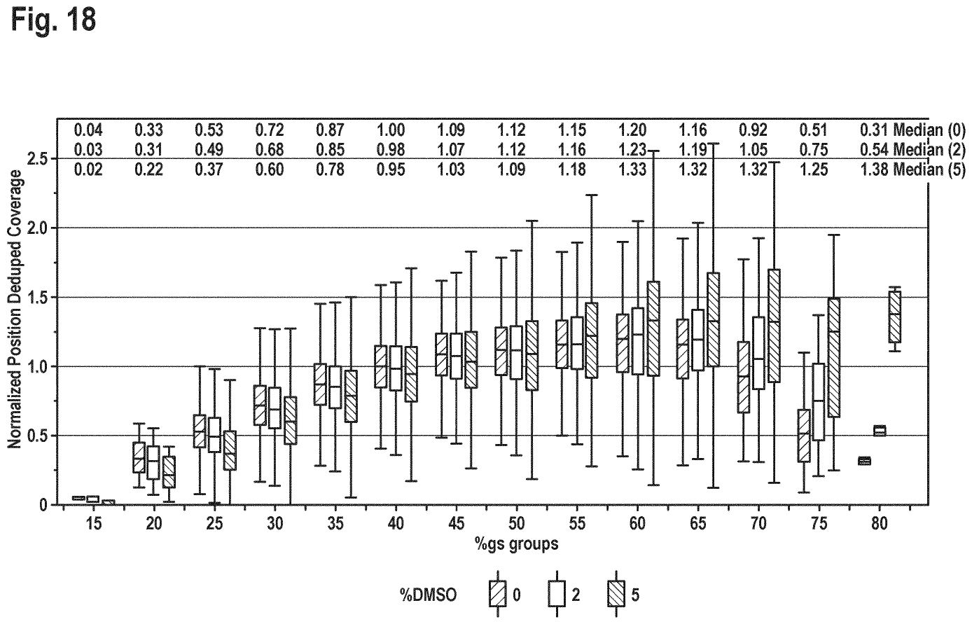

Normalized coverage for higher % GC targets improved with increasing DMSO in the capture extension (see FIG. 18). At 2% DMSO, there was also minimal negative impact on coverage for low GC/high AT targets. Data not shown for 1% and 3%, however the trend was the same.

### Conclusion

The addition of 2% DMSO in the capture extension using the 1.7 Mb panel improved coverage of high % GC targets while minimizing negative impact on low % GC targets and based on the sequencing quality metrics also improved or maintained similar enrichment performance.

### ADDITIONAL EMBODIMENTS

Additional Embodiment 1. A composition comprising: (a) a polymerase, (b) one or more primers, (c) unmodified dNTPs, and (d) at least one enhancer.
Additional Embodiment 2. The composition of additional embodiment 1, wherein the at least one enhancer is selected from the group consisting of betaine, dimethyl sulfoxide (DMSO), a disaccharide, and a single stranded DNA binding protein (SSB).
Additional Embodiment 3. The composition of any one of the preceding additional embodiments, wherein the at least one enhancer is betaine.
Additional Embodiment 4. The composition of additional embodiment 3, wherein a concentration of betaine in the composition ranges from between about 0.2 mM to about 0.8 mM.
Additional Embodiment 5. The composition of additional embodiment 3, wherein a concentration of betaine in the composition ranges from between about 0.3 mM to about 0.6 mM.
Additional Embodiment 6. The composition of additional embodiment 3, wherein a concentration of betaine in the composition is about 0.5 mM.
Additional Embodiment 7. The composition of additional embodiment 2, wherein the at least one enhancer is DMSO.
Additional Embodiment 8. The composition of additional embodiment 7, wherein an amount of DMSO in the composition ranges from between about 1% (v/v) to about 10% (v/v).
Additional Embodiment 9. The composition of additional embodiment 7, wherein an amount of DMSO in the composition ranges from between about 2% (v/v) to about 9% (v/v).
Additional Embodiment 10. The composition of any one of the preceding additional embodiments, wherein the one or more primers have a Tm ranging from between about 57° C. to about 95° C.
Additional Embodiment 11. The composition of any one of the preceding additional embodiments, wherein the one or more primers have a Tm ranging from between about 57° C. to about 85° C.
Additional Embodiment 12. The composition of any one of the preceding additional embodiments, wherein the one or more primers have a Tm ranging from between about 57° C. to about 75° C.
Additional Embodiment 13. The composition of any one of the preceding additional embodiments, wherein the one or more primers have a Tm ranging from between about 57° C. to about 72° C.
Additional Embodiment 14. The composition of any one of the preceding additional embodiments, wherein the one or more primers have a Tm ranging from between about 57° C. to about 69° C.
Additional Embodiment 15. The composition of any one of the preceding additional embodiments, wherein the one or more primers have a Tm ranging from between about 57° C. to about 66° C.
Additional Embodiment 16. The composition of any one of the preceding additional embodiments, wherein the one or more primers have a Tm ranging from between about 57° C. to about 63° C.
Additional Embodiment 17. The composition of any one of the preceding additional embodiments, wherein the one or more primers comprise one or more modified dNTPs.
Additional Embodiment 18. The composition of additional embodiment 17, wherein the one or more modified dNTPs are selected from the group consisting of modified dGTPs and modified dATPs.
Additional Embodiment 19. The composition of any one of the preceding additional embodiments, further comprising one or more modified dNTPs.
Additional Embodiment 20. The composition of additional embodiment 19, wherein a concentration of the one or more modified dNTPs in the composition is about the same as a concentration of the unmodified dNTPs in the composition.
Additional Embodiment 21. The composition of additional embodiment 19, wherein a concentration of the one or more modified dNTPs in the composition ranges from about 0.1 mM to about 0.5 mM.
Additional Embodiment 22. The composition of additional embodiment 19, wherein a concentration of the one or modified dNTPs in the composition ranges from about 0.2 mM to about 0.4 mM.
Additional Embodiment 23. The composition of additional embodiment 19, wherein the one or more modified dNTPs are modified dGTPs.
Additional Embodiment 24. The composition of additional embodiment 23, wherein the modified dGTPs comprise 7-Deaza-2′-deoxyguanosine-5′-Triphosphates.
Additional Embodiment 25. The composition of additional embodiment 19, wherein the one or more modified dNTPs are modified dATPs.
Additional Embodiment 26. The composition of additional embodiment 25, wherein the modified dATPs comprise 2-Amino-2′ deoxyadenosine-5′-Triphosphates.
Additional Embodiment 27. The composition of any one of additional embodiments 1-26, wherein the composition further comprises a divalent cation.
Additional Embodiment 28. The composition of additional embodiment 27, wherein the divalent cation is selected from the group consisting of Co²⁺, Mn²⁺, Mg²⁺, Cd²⁺, and Ca²⁺.
Additional Embodiment 29. The composition of any one of the preceding additional embodiments, wherein the composition further comprises one or more buffers.
Additional Embodiment 30. The composition of any one of the preceding additional embodiments, wherein the composition further comprises one or more polyols.
Additional Embodiment 31. The composition of any one of the preceding additional embodiments, wherein the one or more primers comprise pre-capture forward and reverse primers.
Additional Embodiment 32. The composition of any one of the preceding additional embodiments, wherein the one or more primers comprise capture primers.
Additional Embodiment 33. The composition of any one of the preceding additional embodiments, wherein the capture primers comprise a capture moiety.
Additional Embodiment 34. The composition of any one of the preceding additional embodiments, wherein the composition further comprises input nucleic acid molecules.
Additional Embodiment 35. The composition of additional embodiment 34, wherein the input nucleic acid molecules comprise a library of nucleic acid molecules, where each nucleic acid molecule in the library of nucleic acid molecules comprises first and second adapters.
Additional Embodiment 36. The composition of additional embodiment 34, wherein the one or more primers comprise one or more capture primers, and wherein the one or more capture primers are capable of hybridizing to target nucleic acid sequences within the library of nucleic acid molecules.
Additional Embodiment 37. A composition comprising: (a) a polymerase, (b) one or more primers, (c) dNTPs, and (d) optionally at least one enhancer, wherein the one or more primers include no limitation on the percent of guanine or cytosine bases, and wherein at least one primer of the one or more primers has a melting temperature greater than 63° C.
Additional Embodiment 38. The composition of additional embodiment 37, wherein the at least one primer has a melting temperature greater than about 69° C.
Additional Embodiment 39. The composition of additional embodiment 37, wherein the at least one primer has a melting temperature greater than about 75° C.
Additional Embodiment 40. The composition of additional embodiment 37, wherein the at least one primer has a melting temperature greater than about 85° C.
Additional Embodiment 41. The composition of additional embodiment 37, wherein the at least one primer has a melting temperature greater than about 95° C.
Additional Embodiment 42. The composition of any one of additional embodiments 37-41, wherein the at least one optional enhancer is selected from the group consisting of betaine, DMSO, a disaccharide, and a single stranded DNA binding protein (SSB).
Additional Embodiment 43. The composition of additional embodiment 42, wherein the at least one optional enhancer is betaine, and wherein a concentration of the betaine in the composition ranges from between about 0.2 mM to about 0.8 mM.
Additional Embodiment 44. The composition of any one of additional embodiments 37-43, wherein the dNTPs comprise a mixture of unmodified dNTPs and modified dNTPs.
Additional Embodiment 45. The composition of additional embodiment 44, wherein the one or more modified dNTPs comprise 7-Deaza-2′-deoxyguanosine-5′-Triphosphate.
Additional Embodiment 46. The composition of additional embodiment 44, wherein the one or more modified dNTPs comprise 2-Amino-2′ deoxyadenosine-5′-Triphosphate.
Additional Embodiment 47. The composition of any one of additional embodiments 44-46, wherein a concentration of the one or more modified dNTPs in the composition ranges from between about 0.2 mM to about 0.8 mM.
Additional Embodiment 48. The composition of any one of additional embodiments 37-41, wherein the one or more primers comprise one or more modified dNTPs.
Additional Embodiment 49. The composition of additional embodiment 48, wherein the one or more modified dNTPs are selected from the group consisting of 7-Deaza-2′-deoxyguanosine-5′-Triphosphate and 2-Amino-2′ deoxyadenosine-5′-Triphosphate.
Additional Embodiment 50. The composition of any one of additional embodiments 37-49, wherein the composition further comprises a divalent cation.
Additional Embodiment 51. The composition of additional embodiment 50, wherein the divalent cation is selected from the group consisting of Co2+, Mn2+, Mg2+, Cd2+, and Ca2+.
Additional Embodiment 52. The composition of any one of additional embodiments 37-51, wherein the composition further comprises one or more buffers.
Additional Embodiment 53. The composition of any one of additional embodiments 37-52, wherein the composition further comprises one or more polyols.
Additional Embodiment 54. The composition of any one of additional embodiments 37-53, wherein the one or more primers comprise pre-capture forward and reverse primers.
Additional Embodiment 55. The composition of any one of additional embodiments 37-53 wherein the one or more primers are capture primers.
Additional Embodiment 56. The composition of any one of additional embodiments 37-55, wherein the composition further comprises input nucleic acid molecules.
Additional Embodiment 57. The composition of additional embodiment 56, wherein the input nucleic acid molecules comprise first and second adapters.
Additional Embodiment 58. The composition of additional embodiment 56, wherein the one or more primers comprise one or more capture primers, and wherein the one or more capture primers are capable of hybridizing to target nucleic acid sequences of the nucleic acid molecules.
Additional Embodiment 59. The composition of additional embodiment 56, wherein the input nucleic acid molecules comprise DNA.
Additional Embodiment 60. A reaction tube comprising the composition of any one of additional embodiments 1-59.
Additional Embodiment 61. Use of the composition of any one of additional embodiments 1-33 and 37-55 in the amplification of input nucleic acid molecules.
Additional Embodiment 62. A composition comprising: (a) one or more primers, (b) input nucleic acid molecules, and (c) at least one enhancer selected from the group consisting of betaine, DMSO, a disaccharide, and a single stranded DNA binding protein (SSB).
Additional Embodiment 63. The composition of additional embodiment 62, wherein the at least one enhancer is betaine.
Additional Embodiment 64. The composition of additional embodiment 63, wherein a concentration of betaine in the composition ranges from between about 0.2 mM to about 0.8 mM.
Additional Embodiment 65. The composition of additional embodiment 63, wherein a concentration of betaine in the composition ranges from between about 0.3 mM to about 0.6 mM.
Additional Embodiment 66. The composition of additional embodiment 62, wherein the at least one enhancer is DMSO.
Additional Embodiment 67. The composition of additional embodiment 66, wherein an amount of DMSO in the composition ranges from between about 1% (v/v) to about 10% (v/v).
Additional Embodiment 68. The composition of additional embodiment 66, wherein an amount of DMSO in the composition ranges from between about 2% (v/v) to about 8% (v/v).
Additional Embodiment 69. The composition of additional embodiment 62, wherein the one or more primers have a Tm ranging from between about 57° C. to about 95° C.
Additional Embodiment 70. The composition of additional embodiment 62, wherein the one or more primers have a Tm ranging from between about 57° C. to about 85° C.
Additional Embodiment 71. The composition of additional embodiment 62, wherein the one or more primers have a Tm ranging from between about 57° C. to about 75° C.
Additional Embodiment 72. The composition of additional embodiment 62, wherein the one or more primers have a Tm ranging from between about 57° C. to about 72° C.
Additional Embodiment 73. The composition of additional embodiment 62, wherein the one or more primers have a Tm ranging from between about 57° C. to about 69° C.
Additional Embodiment 74. The composition of additional embodiment 62, wherein the one or more primers have a Tm ranging from between about 57° C. to about 66° C.
Additional Embodiment 75. The composition of additional embodiment 62, wherein the one or more primers comprise one or more modified dNTPs.
Additional Embodiment 76. The composition of additional embodiment 75, wherein the one or more modified dNTPs are selected from the group consisting of modified dGTPs and modified dATPs.
Additional Embodiment 77. The composition of any one of additional embodiments 62-76, wherein the composition further comprises a divalent cation.
Additional Embodiment 78. The composition of additional embodiment 77, wherein the divalent cation is selected from the group consisting of Co²⁺, Mn²⁺, Mg²⁺, Cd²⁺, and Ca²⁺.
Additional Embodiment 79. The composition of any one of additional embodiments 62-78 wherein the composition further comprises one or more buffers.
Additional Embodiment 80. The composition of any one of additional embodiments 62-79, wherein the input nucleic acid molecules comprise a prepared nucleic acid library.
Additional Embodiment 81. The composition of any one of additional embodiments 62-79, wherein the input nucleic acid molecules comprise captured nucleic acid molecules.
Additional Embodiment 82. The composition of additional embodiment 81, wherein the captured nucleic acid molecules comprise a complex of a nucleic acid molecule comprising a target nucleic acid sequence and an extended capture primer hybridized to at least a portion of the target nucleic acid sequence.
Additional Embodiment 83. A composition comprising: (a) input nucleic acid molecules, and (b) one or more primers, and optionally (c) at least one enhancer, wherein the one or more primers include no limitation on the percent of guanine or cytosine bases, and wherein at least one primer of the one or more primers has a melting temperature greater than 63° C.
Additional Embodiment 84. The composition of additional embodiment 83, wherein the at least one primer has a melting temperature greater than about 69° C.
Additional Embodiment 85. The composition of additional embodiment 83, wherein the at least one primer has a melting temperature greater than about 75° C.
Additional Embodiment 86. The composition of additional embodiment 83, wherein the at least one primer has a melting temperature greater than about 85° C.
Additional Embodiment 87. The composition of additional embodiment 83, wherein the at least one primer has a melting temperature greater than about 95° C.
Additional Embodiment 88. The composition of any one of additional embodiments 83-87, wherein the one or more primers comprise one or more modified dNTPs.
Additional Embodiment 89. The composition of additional embodiment 88, wherein the one or more modified dNTPs are selected from the group consisting of 7-Deaza-2′-deoxyguanosine-5′-Triphosphate and 2-Amino-2′ deoxyadenosine-5′-Triphosphate.
Additional Embodiment 90. The composition of any one of additional embodiments 83-89, wherein the at least one optional enhancer is selected from the group consisting of betaine, DMSO, a disaccharide, and a single stranded DNA binding protein (SSB).
Additional Embodiment 91. The composition of additional embodiment 90, wherein the at least one optional enhancer is betaine, and wherein a concentration of the betaine in the composition ranges from between about 0.2 mM to about 0.8 mM.
Additional Embodiment 92. The composition of any one of additional embodiments 83-91, wherein the composition further comprises a divalent cation.
Additional Embodiment 93. The composition of additional embodiment 92, wherein the divalent cation is selected from the group consisting of Co2+, Mn2+, Mg2+, Cd2+, and Ca2+.
Additional Embodiment 94. The composition of any one of additional embodiments 83-93, wherein the composition further comprises one or more buffers.
Additional Embodiment 95. The composition of any one of additional embodiments 83-94, wherein the input nucleic acid molecules comprise a prepared nucleic acid library.
Additional Embodiment 96. The composition of any one of additional embodiments 83-94, wherein the input nucleic acid molecules comprise captured DNA
Additional Embodiment 97. The composition of additional embodiment 96, wherein the captured nucleic acid molecules comprise a complex of a nucleic acid molecule comprising a target nucleic acid sequence and an extended capture primer hybridized to at least a portion of the target nucleic acid sequence.
Additional Embodiment 98. A reaction tube comprising the composition of any one of additional embodiments 62-97.
Additional Embodiment 99. Use of the composition of any one of additional embodiments 62-79 and 83-94 for the preferential enrichment of one or more target nucleic acid molecules in a library of nucleic acid molecules.
Additional Embodiment 100. A kit comprising: (a) a set of capture primers; (b) a set of release primers; (c) a polymerase; (d) dNTPs; and (e) betaine or a derivative or analog thereof.
Additional Embodiment 101. The kit of additional embodiment 100, wherein the dNTPs comprise a mixture of unmodified and modified dNTPs.
Additional Embodiment 102. The kit of additional embodiment 101, wherein the modified dNTPs include modified dGTPs and modified dATPs.
Additional Embodiment 103. The kit of any one of additional embodiments 100-102, wherein the sets of capture and release primers include one or more modified dNTPs.
Additional Embodiment 104. The kit of additional embodiment 100, further comprising instructions to prepare a capture extension master mix, wherein the prepared capture extension master mix includes a concentration of betaine of about 0.5 mM.
Additional Embodiment 105. The kit of any one of additional embodiments 100-104, further comprising one or more buffers
Additional Embodiment 106. The kit of any one of additional embodiments 100-105, further comprising one or more divalent cations.
Additional Embodiment 107. The kit of any one of additional embodiments 100-106, further comprising one or more polyols.
Additional Embodiment 108. The kit of any one of additional embodiments 100-107, wherein at least one of the sets of capture and release primers have no limits on melting temperatures and/or GC content.
Additional Embodiment 109. The kit of any one of additional embodiments 100-108, wherein both sets of capture and release primers have no limits on melting temperatures and/or GC content.
Additional Embodiment 110. A kit comprising: (a) a set of capture primers; (b) a set of release primers; (c) a polymerase; and (d) dNTPs; wherein at least one primer of the set of capture primers has a melting temperature greater than about 65° C.; and wherein at least one primer of the set of release primers has a melting temperature greater than about 65° C.
Additional Embodiment 111. The kit of additional embodiment 110, wherein the at least one primer of the set of capture primers has a melting temperature greater than about 72° C.; and wherein the at least one primer of the set of release primers has a melting temperature greater than about 72° C.
Additional Embodiment 112. The kit of any one of additional embodiments 110-111, wherein the at least one primer of the set of capture primers comprises one or more modified dNTPs; and wherein the at least one primer of the set of release primers comprises one or more modified dNTPs.
Additional Embodiment 113. The kit of any one of additional embodiments 110-112, wherein the kit further comprises at least one enhancer selected from the group consisting of betaine or a derivative or analog thereof, DMSO, a single stranded DNA binding protein, or a disaccharide.
Additional Embodiment 114. A method of producing a capture primer extension complex comprising a target nucleic acid molecule and a capture primer, the method comprising: (a) hybridizing a capture primer to a portion of the target nucleic acid molecule in a library of nucleic acid molecules, where each of the nucleic acid molecules in the library of nucleic acid molecules has a first end comprising a first adapter and a second end comprising a second adapter; and (b) extending the hybridized capture primer with a first polymerase to producing the capture primer extension complex; wherein the capture primer has a melting temperature greater than 63° C.
Additional Embodiment 115. The method of additional embodiment 114, wherein the melting temperature of the capture primer is greater than 66° C.
Additional Embodiment 116. The method of additional embodiment 114, wherein the melting temperature of the capture primer is greater than 72° C.
Additional Embodiment 117. The method of additional embodiment 114, wherein the melting temperature of the capture primer is greater than 75° C.
Additional Embodiment 118. The method of any one of additional embodiments 114-117, wherein the capture primer hybridized to the target nucleic acid molecule is extended in a composition comprising at least one enhancer selected from the group consisting of betaine or a derivative or analog thereof, DMSO, a single stranded DNA binding protein, and a disaccharide.
Additional Embodiment 119. The method of additional embodiment 118, wherein the at least one enhancer is betaine, and wherein the concentration of betaine ranges from between about 0.2 mM to about 0.8 mM.
Additional Embodiment 120. The method of additional embodiment 118, wherein the at least one enhancer is betaine, and wherein the concentration of betaine ranges from between about 0.3 mM to about 0.7 mM.
Additional Embodiment 121. The method of additional embodiment 118, wherein the at least one enhancer is betaine, and wherein the concentration of betaine ranges from between about 0.4 mM to about 0.6 mM.
Additional Embodiment 122. The method of any one of additional embodiments 114-117, wherein the capture primer hybridized to the target nucleic acid molecule is extended in a composition comprising one or more modified dNTPs.
Additional Embodiment 123. The method of additional embodiment 122, wherein the one or more modified dNTPs are modified dGTPs.
Additional Embodiment 124. The method of additional embodiment 123, wherein the modified dGTPs comprise 7-Deaza-2′-deoxyguanosine-5′-Triphosphates.
Additional Embodiment 125. The method of additional embodiment 122, wherein the one or more modified dNTPs are modified dATPs.
Additional Embodiment 126. The method of additional embodiment 125, wherein the modified dATPs comprise 2-Amino-2′ deoxyadenosine-5′-Triphosphates.
Additional Embodiment 127. The method of any one of additional embodiments 114-117, wherein the capture primer hybridized to the target nucleic acid molecule is extended in a composition comprising betaine and one or more modified dNTPs.
Additional Embodiment 128. The method of additional embodiment 127, wherein a concentration of betaine in the composition is about 0.5 mM.
Additional Embodiment 129. The method of any one of additional embodiments 114-128, further comprising capturing the capture primer extension complex.
Additional Embodiment 130. The method of any one of additional embodiments 114-129, further comprising hybridizing a release primer to the target nucleic acid.
Additional Embodiment 131. The method of additional embodiment 130, wherein the release primer is hybridized to the target nucleic acid in a composition comprising at least one enhancer.
Additional Embodiment 132. The method of additional embodiment 131, wherein the at least one enhancer is betaine.
Additional Embodiment 133. The method of additional embodiment 130, wherein the release primer is hybridized to the target nucleic acid in a composition comprising one or more dNTPs.
Additional Embodiment 134. The method of additional embodiment 130, wherein the release primer has a melting temperature greater than 63° C.
Additional Embodiment 135. The method of additional embodiment 134, wherein the melting temperature greater than 65° C.
Additional Embodiment 136. The method of additional embodiment 134, wherein the melting temperature greater than 72° C.
Additional Embodiment 137. The method of additional embodiment 130, further comprising extending the release primer hybridized to the target nucleic acid with a second polymerase.
Additional Embodiment 138. A method of amplifying one or more nucleic acid molecules comprising: (a) obtaining a plurality of nucleic acid molecules; and (b) performing a first amplification reaction in a first composition comprising the obtained plurality of nucleic acid molecules and a first set of primers, wherein at least one primer of the first set of primers has a melting temperature greater than 63° C.
Additional Embodiment 139. The method of additional embodiment 138, wherein each nucleic acid molecule of the obtained plurality of nucleic acid molecules comprises first and second adapters.
Additional Embodiment 140. The method of any one of additional embodiments 138-139, wherein the first composition further comprises betaine.
Additional Embodiment 141. The method of additional embodiment 140, wherein a concentration of betaine in the first composition is about 0.5 mM.
Additional Embodiment 142. The method of additional embodiment 139, further comprising enriching the obtained plurality of nucleic acid molecules for one or more target nucleic acid molecules.
Additional Embodiment 143. The method of additional embodiment 142, further comprising performing a second amplification reaction in a second composition comprising the one or more target nucleic acid molecules and a second set of primers, wherein at least one primer of the second set of primers has a melting temperature greater than 63° C.
Additional Embodiment 144. The method of additional embodiment 143, wherein the second composition further comprises betaine.
Additional Embodiment 145. The method of additional embodiment 144, wherein a concentration of betaine in the second composition is about 0.5 mM.
Additional Embodiment 146. The method of additional embodiment 138, wherein the obtained plurality of nucleic acid molecules is a target enriched library comprising a plurality of target nucleic acid molecules.
Additional Embodiment 147. The method of additional embodiment 146, further comprising sequencing the amplified target enriched library.
Additional Embodiment 148. A method of amplifying one or more nucleic acid molecules comprising: (a) obtaining a plurality of nucleic acid molecules; (b) performing a first amplification reaction in a first composition comprising the plurality of nucleic acid molecules, betaine, and a mixture of unmodified dNTPs and modified dNTPs.
Additional Embodiment 149. The method of additional embodiment 148, wherein a concentration of betaine in the first composition is about 0.5 mM.
Additional Embodiment 150. The method of any one of additional embodiments 148-149, wherein at least one primer of the first set of primers has a high melting temperature and/or a high GC content.
Additional Embodiment 151. The method of additional embodiment 148, wherein each nucleic acid molecule of the obtained plurality of nucleic acid molecules comprises first and second adapters, and wherein the method further comprises enriching the obtained plurality of nucleic acid molecules for one or more target nucleic acid molecules.
Additional Embodiment 152. The method of additional embodiment 151, further comprising performing a second amplification reaction in a second composition comprising the one or more target nucleic acid molecules, betaine, and a mixture of unmodified dNTPs and modified dNTPs.
Additional Embodiment 153. The method of additional embodiment 148, wherein the obtained plurality of nucleic acid molecules is a target enriched library comprising a plurality of target nucleic acid molecules.
Additional Embodiment 154. The method of additional embodiment 153, further comprising sequencing the amplified target enriched library.
Additional Embodiment 155. A kit for enrichment of at least one target nucleic acid in a library of nucleic acid molecules, the kit comprising: (a) a first oligonucleotide complementary to a target nucleic acid in library of nucleic acid molecules, each of the nucleic acid molecules in the library of nucleic acid molecules having a first end comprising a first adapter and a second end comprising a second adapter; (b) a second oligonucleotide complementary to the target nucleic acid; a first amplification primer; and (c) a second amplification primer; and where at least one of the first oligonucleotide or the second oligonucleotide has a high melting temperature and/or a high GC content.
Additional Embodiment 156. The kit of additional embodiment 155, wherein both the first and second oligonucleotides have a high melting temperature and/or a high GC content.
Additional Embodiment 157. A composition, comprising: (a) a library of nucleic acid molecules comprising at least one target nucleic acid, each of the nucleic acid molecules in the library of nucleic acid molecules having a first end comprising a first adapter, a second end comprising a second adapter, and a region of interest intermediate the first adapter and the second adapter; (b) an extended first oligonucleotide hybridized to the region of interest of the target nucleic acid, the extended first oligonucleotide including at least one capture moiety; a solid support bound to the at least one capture moiety; (c) a second oligonucleotide hybridized to the target nucleic acid at a position 5′ to the first extended oligonucleotide; and (d) a polymerase associated with a 3′ end of the second oligonucleotide; wherein at least one of the first oligonucleotide or the second oligonucleotide has a high melting temperature and/or a high GC content.
Additional Embodiment 158. A composition comprising: (a) a polymerase, (b) one or more primers having no limits on melting temperature and/or GC content, (c) a mixture of unmodified dNTPs and modified dNTPs, and (d) at least one enhancer.
Additional Embodiment 159. The composition of additional embodiment 158, wherein the at least one enhancer is betaine.
Additional Embodiment 160. The composition of any one of additional embodiments 158 and 159, further comprising one or more nucleic acid molecules.
Additional Embodiment 161. A composition consisting essentially of a polymerase, one or more primers having no limits on melting temperature and/or GC content, a mixture of unmodified dNTPs and modified dNTPs, and at least one enhancer.
Additional Embodiment 162. The composition of additional embodiment 161, wherein the at least one enhancer is betaine.
Additional Embodiment 163. The composition of any one of additional embodiments 161 and 162, further comprising one or more nucleic acid molecules.
Additional Embodiment 164. A composition consisting of a polymerase, one or more primers having no limits on melting temperature and/or GC content, a mixture of unmodified dNTPs and modified dNTPs, and at least one enhancer.
Additional Embodiment 165. The composition of additional embodiment 164, wherein the at least one enhancer is betaine.
Additional Embodiment 166. The composition of any one of additional embodiments 164 and 165, further comprising one or more nucleic acid molecules.
Additional Embodiment 167. A reaction tube comprising the composition of any one of additional embodiments 158-166.
Additional Embodiment 168. Use of the composition of any one of additional embodiments 158, 159, 161, 162, 164, and 165 in the amplification of input nucleic acid molecules.
Additional Embodiment 169. A method of amplifying one or more nucleic acid molecules, wherein the amplification is performed in the presence of any one of the compositions of additional embodiments 158, 159, 161, 162, 164, and 165.
Additional Embodiment 170. The method of additional embodiment 169, wherein a step of thermocycling is performed for about 34 minutes.

1. A composition comprising:
(a) a polymerase;
(b) one or more primers;
(c) unmodified dNTPs;
(d) at least one enhancer selected from the group consisting of betaine, dimethyl sulfoxide (DMSO), a trehalose, and a single-stranded DNA binding protein; and
(e) optionally one or more modified dNTPs.

2. The composition of claim 1, wherein the at least one enhancer is DMSO, and wherein an amount of DMSO in the composition ranges from between about 1% (v/v) to about 3% (v/v).

3. The composition of claim 1, wherein the at least one enhancer is betaine, and wherein a concentration of betaine in the composition ranges from between about 0.4 mM to about 0.6 mM.

4. The composition of claim 1, wherein the at least one enhancer is trehalose, and wherein a concentration of trehalose in the composition is about 0.1M.

5. The composition of claim 1, wherein the at least one enhancer is the single-stranded binding protein, and wherein a concentration of the single-stranded binding protein in the composition ranges from between about 1 ng/ml to about 9 ng/mL.

6. The composition of claim 1, wherein the one or more primers is a capture primer, and wherein the capture primer includes a capture moiety.

7. The method of claim 1, wherein at least two enhancers are present in the composition.

8. A composition comprising:
(a) a polymerase;
(b) one or more primers;
(c) a dNTP mixture, wherein the dNTP mixture comprises: (1) unmodified dNTPs, and (2) modified dNTPs, wherein the modified dNTPs are selected from a group consisting of: (i) 7-deaza dGTP, (ii) 2-amino dATP, and (iii) 7-deaza dGTP and 2-amino dATP, and
(d) optionally at least one enhancer selected from the group consisting of: betaine or a derivative or analog thereof, DMSO, a single-stranded DNA binding protein, and a disaccharide,
wherein the one or more primers include no limitation on the percent of guanine or cytosine bases, and
wherein at least one primer of the one or more primers has a melting temperature greater than 63° C.

9. The composition of claim 8, wherein the modified dNTPs comprise both 7-deazas dGTP and 2-amino dATPs, and wherein the 7-deaza dGTPs and 2-amino dATPs are each present in a concentration ranging from between about 0.2 mM to about 0.4 mM.

10. A method of producing a capture primer extension complex comprising a target nucleic acid molecule and a capture primer, wherein the method comprises the following steps:
(a) hybridizing a capture primer to a portion of the target nucleic acid molecule in a library of nucleic acid molecules, wherein each of the nucleic acid molecules in the library of nucleic acid molecules has a first end, wherein the first end comprises a first adapter, and a second end, wherein the second end comprises a second adapter; and
(b) extending the hybridized capture primer with a first polymerase, to produce the capture primer extension complex, wherein the capture primer hybridized to the target nucleic acid molecule is extended in a composition comprising at least one enhancer selected from the group consisting of: betaine or a derivative or analog thereof, DMSO, a single-stranded DNA binding protein, and a disaccharide.

11. The composition of claim 10, wherein the at least one enhancer is DMSO, and wherein an amount of DMSO in the composition ranges from between about 1% (v/v) to about 3% (v/v).

12. The composition of claim 10, wherein the at least one enhancer is betaine, and wherein a concentration of betaine in the composition ranges from between about 0.4 mM to about 0.6 mM.

13. The composition of claim 10, wherein the at least one enhancer is trehalose, and wherein a concentration of trehalose in the composition is about 0.1 M.

14. The composition of claim 10, wherein the at least one enhancer is the single-stranded binding protein, and wherein a concentration of the single-stranded binding protein in the composition ranges from between about 21 ng/mL to about 9 ng/mL.

15. The method of claim 10, wherein the composition comprises at least two enhancers.

16. A method of amplifying one or more nucleic acid molecules, wherein the method comprises the following steps:
(a) obtaining a plurality of nucleic acid molecules;
(b) performing a first amplification reaction, to amplify the obtained plurality of nucleic acid molecules, wherein the first amplification reaction is performed using a first composition, wherein the first composition comprises:
(i) a polymerase;
(ii) one or more primers;
(iii) a dNTP mixture, wherein the dNTP mixture comprises: (1) unmodified dNTPs, and (2) modified dNTPs, wherein the modified dNTPs are selected from a group consisting of: (A) 7-deaza dGTP, (B) 2-amino dATP, and (C) 7-deaza dGTP and 2-amino dATP; and
(iv) optionally at least one enhancer selected from a group consisting of: betaine or a derivative or analog thereof, DMSO, a single-stranded DNA binding protein, and a disaccharide; and
wherein the one or more primers includes no limitation on the percent of guanine or cytosine bases, and wherein at least one primer of the one or more primers has a melting point greater than 63° C.

17. The composition of claim 16, wherein the modified dNTPs comprise both 7-deazas dGTP and 2-amino dATPs, and wherein the 7-deaza dGTPs and 2-amino dATPs are each present in a concentration ranging from between about 0.2 mM to about 0.4 mM.

18. The method of claim 16, further comprising a step of enriching the obtained plurality of nucleic acid molecules for one or more target nucleic acid molecules prior to performing the first amplification reaction.

19. The method of claim 16, further comprising a step of performing a second amplification reaction, wherein the second amplification is performed using a second composition, wherein the second composition comprises the following:
(i) a polymerase;
(ii) one or more primers;
(iii) a dNTP mixture, wherein the dNTP mixture comprises: (a) unmodified dNTPs, and (b) modified dNTPs, wherein the modified dNTPs are selected from the group consisting of: (1) 7-deaza dGTP, (2) 2-amino dATP, and (3) 7-deaza dGTP and 2-amino dATP; and
(iv) optionally at least one enhancer selected from the group consisting of betaine or a derivative or analog thereof, DMSO, a single-stranded DNA binding protein, and a disaccharide.
